(19) United States
(12) Patent Application Publication
(10) Pub. No.: US 2026/0176701 A1
(43) Pub. Date: Jun. 25, 2026

(54) ULTRASENSITIVE ASSAYS FOR DETECTION OF ORF1P IN BIOFLUIDS

(71) Applicants: The Brigham and Women's Hospital, Inc., Boston, MA (US); The General Hospital Corporation, Boston, MA (US); Dana-Farber Cancer Institute, Inc., Boston, MA (US); The Rockefeller University, New York, NY (US); The Johns Hopkins University, Baltimore, MD (US)

(72) Inventors: Martin Taylor, Newton, MA (US); Connie Wu, Boston, MA (US); Peter Clayton Fridy, New York, NY (US); John Paul LaCava, New York, NY (US); Brian T. Chait, New York, NY (US); Kelly Rebecca Molloy, New York, NY (US); Michael Paul Rout, New York, NY (US); Padric Garden, Boston, MA (US); Limor Cohen, Boston, MA (US); David R. Walt, Boston, MA (US); Kathleen Burns, Chestnut Hill, MA (US)

(21) Appl. No.: 19/127,521

(22) PCT Filed: Nov. 8, 2023
(86) PCT No.: PCT/US2023/079148

§ 371 Date: May 6, 2025

Related U.S. Application Data
(60) Provisional application No. 63/423,696, filed on Nov. 8, 2022.

(51) Int. Cl. C12Q 1/6886

(52) U.S. Cl. CPC C12Q 1/6886; C07K 2317/24

(57) ABSTRACT

Described herein are methods and compositions for accurate detection of cancer using ultrasensitive immunoassays, e.g., digital ELISA, to detect open reading frame 1 protein (ORF1p), which is encoded by the LINE-1 retrotransposon, in biofluids.

### CLAIM OF PRIORITY

This application claims the benefit of U.S. Provisional Patent Application Ser. No. 63/423,696, filed on Nov. 8, 2022. The entire contents of the foregoing are hereby incorporated by reference.

### TECHNICAL FIELD

Described herein are methods and compositions for accurate detection of cancer using ultrasensitive immunoassays, e.g., digital ELISA, to detect open reading frame 1 protein (ORF1p), which is encoded by the LINE-1 retrotransposon, in biofluids.

### BACKGROUND

Early detection of cancer is critical for improving outcomes. For example, ovarian cancer is the fifth leading cause of cancer-related deaths among women in the U.S., as it is predominantly diagnosed in advanced stages, with high-grade serous ovarian cancer (HGSOC) accounting for 70-80% of ovarian cancer deaths¹, but a 5-year survival rates of >90% for cases diagnosed in Stage I². Although circulating tumor DNA (ctDNA) has shown great promise for cancer detection, the extremely low, often undetectable levels of ctDNA during early disease stages remains a key obstacle in ovarian³ and other cancers. MicroRNAs (miRNAs) have also demonstrated great potential, but further work in clinical validation of miRNA signatures for early cancer detection is required. Proteins are a promising class of biomarkers, as they are the direct functional players in biological processes and can exist at higher abundances in blood compared to ctDNA. However, protein-based liquid biopsies remain limited by severe gaps in biomarker specificities and protein measurement technologies. For instance, in the case of ovarian cancer, while the FDA has approved the blood-based biomarkers carbohydrate antigen 125 (CA125) and human epididymis protein 4 (HE4), they have poor sensitivity and specificity for early detection, limiting their utility for screening⁴.

### SUMMARY

Provided herein are methods comprising obtaining a sample comprising blood from a subject, e.g., a subject who is suspected or at risk of having cancer, and determining a level of ORF1p in the sample using an ultrasensitive protein assay (i.e., an assay having a limit of detection under 1 picomolar (0.1 femtomoles in 100 ul). In some embodiments, the methods further include comparing the level of ORF1p in the sample to a disease reference, wherein a level of ORF1p above the reference indicates that the subject has or is at risk of developing cancer.

In some embodiments, the cancer is a carcinoma, e.g., ovarian, breast, liver, colon/colorectal, lung, esophageal, prostate, gastric, head and neck, soft tissue, kidney, gallbladder, bile duct (cholangiocarcinoma), bladder, uterine or pancreatic cancer; in some embodiments, the cancer is a carcinoma that is not a brain carcinoma. In some embodiments, the cancer is ovarian cancer, e.g., high-grade serous ovarian cancer (HGSOC). In some embodiments, the cancer is not breast cancer. In some embodiments, the cancer is ovarian, breast, liver, colon/colorectal, lung, esophageal, prostate, gastric, head and neck, brain (optionally glioblastoma), soft tissue, kidney, gallbladder, bile duct (cholangiocarcinoma), bladder, uterine, or pancreatic cancer, blood or bone marrow (optionally lymphoma, leukemia, or myeloma), or skin cancer (optionally melanoma).

Provided herein are methods comprising obtaining a sample comprising blood from a subject, and determining a level of ORF1p in the sample with an ultrasensitive protein assay. In some embodiments, the methods further comprise comparing the level of ORF1p to a disease reference, wherein a level of ORF1p above the reference indicates that the subject has or is at risk of developing cancer. In some embodiments, the sample is a biofluid is whole blood, plasma, or serum; alternatively, in some embodiments, the sample is or comprises stool, cervical fluids such as pap smears, uterine lavage, urine, or sputum. The sample can also be a tissue sample, e.g., from a biopsy (e.g., punch, needle, or shave biopsy, or surgical biopsy), e.g., tissue lysates.

In some embodiments, the cancer is a carcinoma. In some embodiments, the carcinoma is ovarian, breast, liver, colon/colorectal, lung, esophageal, prostate, gastric, head and neck, brain, soft tissue, kidney, gallbladder, bile duct (cholangiocarcinoma), bladder, uterine, or pancreatic cancer. In some embodiments, the cancer is not a brain carcinoma. In some embodiments, the ovarian cancer is high-grade serous ovarian cancer (HGSOC). In some embodiments, the cancer is not breast cancer. In some embodiments, the cancer is of origin in blood, bone marrow, brain, skin, or soft tissue in origin, especially lymphoma, leukemia, myeloma, glioblastoma, or melanoma.

In some embodiments, the ultrasensitive assay is Single-Molecule Arrays (SIMOA); Molecular On-bead Signal Amplification for Individual Counting (MOSAIC); Meso Scale Discovery (MSD); Single-Molecule Counting (SMC); nucleic acid linked immune-sandwich assay (NULISA); LUMINEX; SOMAscan Assays; mass spectrometry (e.g., MALDI-MS), and/or mass cytometry (e.g., CyTOF).

In some embodiments, determining a level of ORF1p comprises contacting the sample with a capture or detection reagent comprising a nanobody selected from Nb2, Nb5, Nb9, Nb10, or NB21 or an ORF1p-binding derivative comprising CDRs thereof (as shown in Table B), or a concatemer thereof, optionally MT1032, MT1033, MT1034, MT1035, MT1036, MT1037, MT1038, MT1039, or MT1040 and/or a monoclonal antibody selected from 62H12, 64C6, 33A8, 61A11, 36D12, 34H7, 50E9, 34C5, 55A6, 42D10, 4H1, Ab6 (ab246317, Abcam), Ab54 (ab246320, Abcam) or an antigen-binding fragment thereof, optionally wherein the capture/detection reagents are 34H7/Ab6, 62H12/Ab6, 34H7/Nb5-5LL, 62H12/Nb5-5LL, 4H1/Nb5-5, or 4H1/Nb5-5LL.

In some embodiments, the nanobody comprises a sequence at least 80%, 85%, 90%, or 95% identical to a sequence in Table B, or a multimer thereof, preferably wherein the CDRs of the nanobody are identical to those from a sequence in Table B, or a multimer thereof. Exemplary nanobody concatemers include MT1032, MT1033, MT1034, MT1035, MT1036, MT1037, MT1038, MT1039, and MT1040 (see FIG. 22A).

In some embodiments, the methods further comprise recommending or sending the subject for additional evaluation, e.g., by imaging and/or biopsy. In some embodiments, the methods further comprise administering a treatment for cancer to a subject who has been identified as having or at risk of developing cancer. In some embodiments, the treatment comprises chemotherapy, hormone therapy, immunotherapy, radiation, or surgical resection.

In some embodiments, the methods further comprise determining a level of ORF1p in the subject after administration of the treatment, and comparing the level of ORF1p prior to treatment with the level of ORF1p during and/or after treatment, wherein a decrease in the level of ORF1p indicates that the treatment is effective in treating the cancer. Thus the methods can be used for monitoring efficacy of treatment. If a treatment is effective, the methods can include continuing the treatment. If a treatment is not effective (e.g., the level of ORF1p does not decrease, or increases), the methods can include selecting and optionally administering a different treatment.

Also provided herein are single domain antibodies or antigen-binding fragments thereof that bind to human ORF1p, as described herein, e.g., comprising a sequence at least 90%, 95%, 97%, or 99% identical to a nanobody sequence or CDR1, CDR2, and CDR3 therefrom as shown in table B, or comprising CDR1, CDR2, and CDR3 as shown in Table B, and multimers thereof.

Additionally provided herein are fusion constructs comprising at least two, e.g., three, four, or five, of the single domain antibodies or antigen-binding fragments thereof as described herein, optionally with linkers therebetween, optionally as shown in Table C. Exemplary nanobody concatemers include MT1032, MT1033, MT1034, MT1035, MT1036, MT1037, MT1038, MT1039, and MT1040 (see FIG. 22A).

Further, provided herein antibodies or antigen binding portions thereof that specifically binds to human ORF1p, as described herein, e.g., wherein the antibody or antigen binding portion thereof comprises at least one of: a heavy chain variable region (VH) comprising or consisting of a VH sequence that is at least 95% identical to a sequence shown in Table D or FIGS. 20A-J, or CDR1, CDR2, and CDR3 therefrom; and/or a light chain variable region (VL) comprising or consisting of a VL sequence that is at least 95% identical to a sequence shown in Table D or FIGS. 20A-J, or CDR1, CDR2, and CDR3 therefrom, preferably wherein the VH and VL or CDRs are from the same antibody.

In some embodiments, the antibody comprises a constant region, optionally as shown in Table A.

In some embodiments, the single domain antibody or antigen-binding fragment thereof, the fusion protein, or the antibody or antigen binding portion thereof, is fused to a tag, e.g., an oligonucleotide, peptide, chemiluminescent, fluorescent, radioactive, or colorimetric label. In some embodiments, the radiolabel is ¹²⁵I.

Also provided herein are nucleic acid molecules encoding the single-domain antibody or antigen-binding fragment thereof, fusion construct, or the antibody or antigen binding portion thereof, as described herein, as well as vectors comprising the nucleic acid molecules, and optionally a promoter, and host cells comprising the nucleic acid molecule, and optionally expressing the single-domain antibody or antigen-binding fragment thereof, the fusion construct, or the antibody or antigen binding portion thereof, as described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Mobilization of the LINE-1 retrotransposon: (i) RNA pol II-mediated transcription of LINE-1 RNA; (ii) translation of open reading frame 1 protein (ORF1p) and ORF2p, and ribonucleoprotein assembly; (iii) insertion of LINE-1 cDNA into the genome via ORF2p-mediated target primed reverse transcription (TPRT) of LINE-1 RNA.

FIG. 2. Signal-to-background evaluation of the indicated pairs of capture (x-axis) and detection (y-axis) antibodies evaluated in samples spiked with recombinant protein (top) or in breast cancer cell lysates (bottom). Darker color indicates higher signal-to-background.

FIG. 3. Representative calibration curves for several affinity reagent pairs (capture/detection).

FIG. 4. Antibody pair screening in breast cancer serum samples (8× dilution).

FIG. 5. Antibody pair screening in BioIVT serum samples (4× dilution).

FIG. 6. Simoa assay measurements of plasma ORF1p levels in patients with high grade serous ovarian cancer (HGSOC) as compared to healthy controls. Two different combinations of capture and detection antibodies are noted above (Ab54/Ab6 and C5/Ab6).

FIGS. 7A-B. A, SIMOA-measured plasma ORF1p levels of a preliminary pan-cancer pilot study; 25 μL each was measured in triplicate using Nb5 (clone 5) nanobody (capture)/Ab6 (detector). Percent detectable is indicated in the pie charts above. Of the 400 “healthy” patients assayed, four were positive; one of those was found to have prostate cancer, limited information is available about the other patients, giving >99% specificity. B, Pilot in high-grade serous ovarian cancer (HGSOC) and healthy patients (Penn cohort).

FIG. 8. Schematic illustration of an exemplary ultrasensitive Simoa assay for ORF1p detection in biofluids.

FIGS. 9A-D. Digital ELISA based on arrays of femtoliter-sized wells (11). (a, b) Single protein molecules are captured and labeled on beads using standard ELISA reagents (a), and beads are loaded into femtoliter-volume well arrays (b). (c) SEM of a section of a femtoliter-volume well array after bead loading. (d) Fluorescence image of a section of the femtoliter-volume well array after signals from single enzymes are generated. Only a fraction of beads possess enzyme activity, indicating a single, bound protein molecule.

FIG. 10A-E. Improved detection of ORF1p with second-generation assays. (A) Schematic of affinity reagents used. 34H7 and 62H2 are custom mAbs; Nb5-5LL is an engineered homodimeric nanobody. (B) 25 μL of plasma from ovarian cancer patients (Penn cohort) was measured in triplicate in our 1st and 2nd gen. assays; affinity reagents used are labeled [capture::detection]. 2nd gen. assays include novel capture reagents (mAbs 34H7 or 62H12) and detection reagent (engineered dimeric nanobody Nb5-5LL). ORF1p is detected in 4 of 5 Stage I patients in the cohort; assay #3 appears to increase sensitivity but may have reduced specificity. (C) 2nd gen. assays were run in triplicate using 25 μL of plasma from MGH advanced-stage gastroesophageal and ovarian cancer cohorts. Ovarian include a mix of 102 HGSOC and 30 other ovarian malignancies (mucinous, clear cell, low-grade serous). 80-90% of ovarian cancers were detectable and sensitivity is increased in both cancer types. (D) 34H7::Nb5-5LL second-generation assay measurements across a multi-cancer cohort. (E) ROC curves with single marker ORF1p across all healthy and ovarian cancer patients (top, n=128-132 cancer, 447-455 healthy), and multivariate models for ovarian (bottom, n=51-53 cancer, 50 healthy).

FIGS. 11A-C. ORF1p is an early predictor of response in 19 gastroesophageal (GE) patients undergoing chemo/chemoradiotherapy and is prognostic in GE and colorectal cancers (CRC). Responders and Non-Responders were characterized retrospectively by medical oncologists blinded to the assays results by post-therapy, pre-surgery imaging. (A) Plasma ORF1p as measured by all three second-generation Simoa assays before and during/post treatment; left panel: Non-Responders have higher pre-treatment ORF1p than Responders (p=0.02, t-test); right panel: ORF1p pre- and on/post therapy classifies Responders and Non-Responders; p<0.0001, Fisher's exact test. (B) Representative CT and PET-CT from patients in the cohort. The representative Non-Responder has the second-highest plasma ORF1p pre-treatment (25.8 pg/ml), which increased to 43.0 pg/ml at day 28 of FOLFOX therapy (47 days after diagnosis), concomitant with increased sizes and number of hepatic metastases seen on CT at day 61. The representative Responder has the fourth-highest plasma ORF1p value in the cohort of Responders (0.83 pg/ml), which decreased to undetectable at day 26 of CROSS therapy (48 days after diagnosis); the displayed PET-CT is 59 days after initiation of therapy, 31 days after the second ORF1p measurement. (C) Kaplan-Meier survival analysis of patients categorized as plasma ORF1p-high and ORF1p-low based on the median plasma ORF1p assay value shows significantly longer survival for ORF1p-low patients with GE (stages III-IV, p=0.0017, log rank test) and CRC (all stage IV, p=0.011, log rank test). Shaded regions represent 95% confidence intervals.

FIG. 12. Pilot larger-volume 2^{nd} generation assay using a flow cytometry-based digital ELISA platform, MOSAIC. A pilot cohort of 10 healthy and 10 gastroesophageal (GE) cancer patients who had levels of ORF1p that were undetectable in 25 μL assay volumes (left panel) were assayed using 20-fold more plasma and readout by flow cytometry, resulting in separation of 9 out of 10 from healthy subjects (right panel).

FIG. 13. Signal-to-Noise Ratios (SNR) by Simoa of novel rabbit monoclonal a-ORF1p antibodies show up to 5-fold improvement vs. our current best antibody, Abcam Ab6, at left. Two different capture beads with distinct epitopes were employed, nanobody C5 (Nb-5) and 4H1.

FIG. 14. Nanobody/antibody pair screening in plasma samples from healthy and cancer (colorectal and gastroesophageal) patients. Capture/Detector pair indicated on each panel. The first-generation assay (Nb5/Ab6) measurements are depicted for comparison. All assays were performed as three-step Simoa assays.

FIG. 15. Screening of newly developed monoclonal antibodies (GenScript) with a dimeric nanobody and commercially available monoclonal antibodies for ORF1p detection with Simoa. Signal-to-background comparisons of affinity reagents as capture/detector pairs on Simoa, using recombinant ORF1p protein. All labeled affinity reagents except Nb5-5(LL) are monoclonal antibodies; Nb5-5(LL) denotes a homodimeric form of the nanobody Nb5.

FIG. 16. Plasma screening round #3: Screening of newly developed monoclonal antibody and dimeric nanobody reagent pairs in plasma from eight healthy and eight cancer (colorectal or gastroesophageal) patients.

FIG. 17. Plasma screening round #4: Screening of newly developed monoclonal antibody and dimeric nanobody reagent pairs in plasma from eight healthy and eight cancer (colorectal or gastroesophageal) patients.

FIG. 18. Plasma screening round #5: Screening of newly developed monoclonal antibody and dimeric nanobody reagent pairs in patient plasma. Affinity reagent pairs selected from previous rounds of screening (FIGS. 16-17) were screened in 25 healthy and 25 cancer (colorectal, gastroesophageal, and breast) patient plasma samples. Each assay is denoted by the capture/detector reagent pair. The first-generation assay (Nb5/Ab6) measurements are depicted for comparison.

FIGS. 19A-C. A, second generation assay 1, 34H7/Nb5-5LL; B, second generation assay 2, 62H12/Nb5-5LL; C, second generation assay 3, 62H12/Ab6.

FIGS. 20A-J. Sequences of monoclonal antibodies shown in Table D.

FIGS. 21A-D. Improved detection of ORF1p with third-generation Simoa assays and with MOSAIC assays. (A) Comparison of 2^{nd} and 3^{rd} generation Simoa assays (25 μL) in 25 mostly undetectable gastroesophageal (GE) cancer and healthy control patients. (B) Schematic of MOSAIC assays. Captured single molecule “immunosandwiches” are formed analogously to Simoa assays. DNA-conjugated streptavidin enables rolling circle amplification to be carried out, generating a strong local fluorescent signal on the bead surface, and then “on” and “off” beads are quantified by flow cytometry, allowing efficient sampling of larger numbers of capture beads. This results in improved sensitivity and multiplexing capabilities. (C) 37H7::Nb5-5LL MOSAIC and Simoa assays in 10 previously-undetectable GE cancer and healthy control patients. Dashed line in left panel and bottom dashed line in right panel indicate analytical limit of detection (LoD) for recombinant ORF1p in buffer. Top dashed line in right panel indicates plasma-specific background in large volume MOSAIC assays, which was used to determine positivity in the pie-charts. (D) Similar results were seen in a breast cancer cohort.

FIGS. 22A-B. Engineered Nanobody constructs. (A) Schematic of design of engineered dimeric and trimeric nanobody constructs, with flexible (GGGGS×4) and rigid helical (EAAAK×3 or DAAAR×3) linker. 5×Cys tag sequence is CGSGRCGSGRCGSGRCGSGRC. (B), Representative preparation of engineered nanobody constructs, Coomassie stain.

FIG. 23. Calibration curves for “3^{rd} Generation” Simoa assays. Two “2^{nd} Generation” assays are compared, gray boxes. Dashed line indicates the assay limit of detection.

FIG. 24. Second round of screening of newly developed “3^{rd} generation” assays using dimeric nanobody detectors. Affinity reagent pairs selected from a first round of screening in plasma samples were screened in 25 healthy and 25 GE cancer patient plasma samples. Each assay is denoted by the capture/detector reagent pair. Second-generation assays were run for comparison (top left and bottom left graphs). Dashed lines indicate assay limits of detection, accounting for four-fold dilution. Middle row of graphs indicate the final three selected third-generation assays. MT1032 and MT1035 are Nb5-5 homodimers with different linkers. MT1036 is a Nb2-Nb9 heterodimer, MT1037 a Nb5-Nb9 heterodimer, and MT1038 a Nb9-Nb9 homodimer.

FIG. 25. Calibration curve for the large-volume (500 μL) MOSAIC assay used in FIG. 5. Dashed line indicates the assay limit of detection.

### DETAILED DESCRIPTION

Liquid biopsies are highly desirable, as they are minimally invasive and can facilitate widespread screening. Many current liquid biopsies detect circulating tumor DNA (ctDNA), circulating tumor cells (CTCs), or microRNAs (miRNAs). However, key challenges remain: (1) the extremely low levels of ctDNA and CTCs during early disease stages are often undetectable and require large blood volumes^{3, 12, 13}; (2) DNA mutations not associated with malignancies can reduce the specificities of ctDNA tests^{14, 15}; (3) while miRNAs have shown promise for the detection of cancers such as ovarian cancer, further clinical validation of miRNA signatures for early detection is needed⁴.

### LINE-1 Retrotransposon Overexpression is a Hallmark of Multiple Human Cancers.

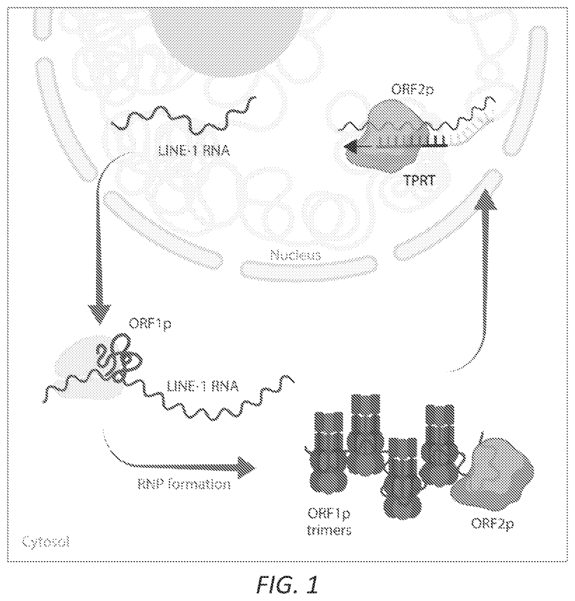

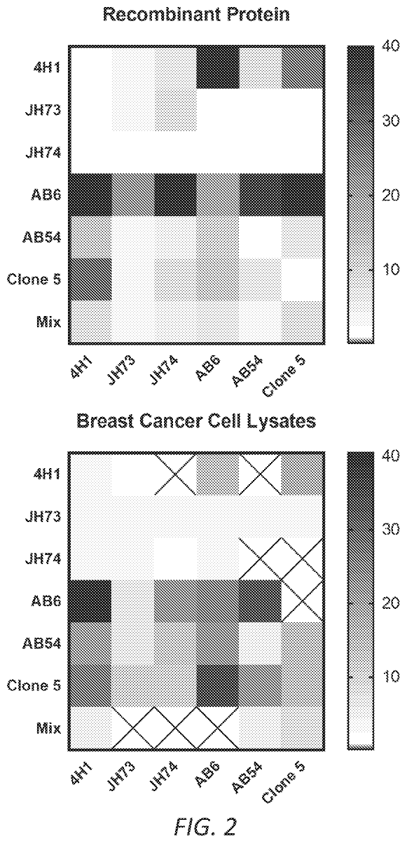

Transposable elements, which comprise nearly half the human genome, have received increasing interest due to their aberrant activities in human cancers⁷. In particular, the expression of the retrotransposon long interspersed element-1 (LINE-1) has emerged as a hallmark of multiple human malignancies and is associated with specific mutational signatures in cancer genomes¹⁷⁻²⁰. LINE-1 is the only protein-coding transposable element still active in humans: its 6-kilobase sequence encodes two proteins essential for retrotransposition, open reading frame 1 protein (ORF1p) and ORF2p (FIG. 1). While LINE-1 expression in somatic cells is normally repressed via promoter methylation and histone modifications, LINE-1 promoter hypomethylation is observed in many human malignancies^{7, 17, 21}. Consistent with this observation, ORF1p is expressed in many tumors, with particularly elevated levels in ovarian cancer^{8, 10, 22} and esophageal cancer^{18,43,44}. Importantly, ORF1p is a stable homotrimer and is highly expressed once derepressed. Taking ovarian cancer as an example, ORF1p expression is observed in over 90% of cases of high-grade serous ovarian cancer (HGSOC), the most aggressive and lethal ovarian cancer subtype⁸. ORF1p is an especially promising “binary” biomarker for ovarian cancer: while ORF1p is not expressed in the normal fallopian tube epithelium, activation of its expression occurs in early precursor lesions of ovarian cancer (serous tubal intraepithelial carcinoma (STIC) lesions)^{9, 10} suggesting its potential utility for early ovarian cancer detection (FIG. 2A). However, the presence of ORF1p in blood remains a largely unexplored opportunity for liquid biopsies. Importantly, the absence of ORF1p expression in non-malignant cells suggests its potential as a “binary” blood-based biomarker, with much higher specificity compared to existing protein cancer biomarkers, which often have varying individual baseline levels and are expressed in normal tissues²³⁻²⁵.

Despite the elevated expression of ORF1p in tumor tissue, ORF1p shed from tumors is diluted in the bloodstream to very low levels, well below the detection limits of conventional methods including mass spectrometry, necessitating ultrasensitive detection. Ultrasensitive single molecule detection technology, SIngle MOlecule Arrays (SIMOA, FIG. 2D), and recently reported detection of ORF1p in the blood^{26,48}.

### Methods of Diagnosis

Included herein are methods for diagnosing cancer. The methods rely on detection of ORF1p in biofluids (e.g., whole blood, plasma or serum, stool, cervical fluids such as pap smears, uterine lavage, urine, or sputum) or tissue samples, e.g., from a biopsy (e.g., punch, needle, or shave biopsy, or surgical biopsy of a suspected cancerous tissue), e.g., tissue lysates, as described herein. In some embodiments, the present methods provide blood tests for cancer detection and diagnosis using circulating ORF1p. In some embodiments, the cancer is a carcinoma, e.g., ovarian, breast, liver, colon/colorectal, lung, esophageal, prostate, gastric, head and neck, brain, soft tissue, kidney, gallbladder, bile duct (cholangiocarcinoma), bladder, uterine, or pancreatic cancers^{8, 22}. In some embodiments, the cancer is of origin in blood, bone marrow, brain, skin, or soft tissue in origin, especially lymphoma, leukemia, myeloma, glioblastoma, or melanoma. In some embodiments, the biofluid is whole blood, plasma, or serum.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, brain, soft tissue, gastrointestinal, and genito-urinary tract, as well as adenocarcinomas which include malignancies such as most colon and colorectal cancers, kidney or renal-cell carcinoma, gallbladder, bile duct (cholangiocarcinoma), bladder, uterine, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, kidney, gallbladder, bile duct (cholangiocarcinoma), bladder, uterine, colon/colorectal and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

In some embodiments, the cancer is a non-brain carcinoma.

The term “sarcoma” is art recognized and refers to malignant tumors of mesenchymal derivation. In some embodiments, the cancer is not sarcoma.

In some embodiments, the cancer is ovarian cancer, e.g., high-grade serous ovarian cancer. In some embodiments, the cancer is not breast cancer.

In some embodiments, the cancer is of origin in blood, bone marrow, brain, skin, or soft tissue in origin, especially lymphoma, leukemia, myeloma, glioblastoma, or melanoma.

An exemplary sequence for human ORF1p is:

MGKKQNRKTGNSKTQSASPPPKERSSSPATEQSWMENDFDELREEGFRRS
NYSELREDIQTKGKEVENFEKNLEECITRITNTEKCLKELMELKTKAREL
REECRSLRSRCDQLEERVSAMEDEMNEMKREGKFREKRIKRNEQSLQEIW
DYVKRPNLRLIGVPESDVENGTKLENTLQDIIQENFPNLARQANVQIQEI
QRTPQRYSSRRATPRHIIVRFTKVEMKEKMLRAAREKGRVTLKGKPIRLT
ADLSAETLQARREWGPIFNILKEKNFQPRISYPAKLSFISEGEIKYFIDK
QMLRDFVTTRPALKELLKEALNMERNNRYQPLQNHAKM* (derived
from Homo sapiens retrotransposon L1 insertion in
X-linked retinitis pigmentosa locus, complete
sequence, GenBank: AF148856.1).

The methods include obtaining a sample from a subject and evaluating the presence and/or level of ORF1p in the sample. In some embodiments, the subject is a mammal, e.g., a human or non-human veterinary subject, e.g., cat, dog, cow, horse, goat, or non-human primate. In some embodiments, the subject is suspected or at risk of having cancer, e.g., has one or more clinical symptoms associated with cancer, or has a family or personal history of cancer, genetic or environmental risk factors for cancer, or has an increased risk of developing cancer as compared to a reference cohort of subjects.

As used herein the term “sample”, when referring to the material to be tested for the presence of ORF1p using a method as described herein, includes inter alia a biofluid, e.g., whole blood, plasma, or serum. In some embodiments, the sample is or comprises stool, cervical fluids such as pap smears, uterine lavage, urine, or sputum. The sample can also be a tissue sample, e.g., from a biopsy (e.g., punch, needle, or shave biopsy, or surgical biopsy); for example tissue lysates can be used. If needed, various methods are well known within the art for the identification and/or isolation and/or purification of ORF1p protein from a sample. An “isolated” or “purified” biological marker such as ORF1p is substantially free of cellular material or other contaminants from the cell or tissue source from which the biological marker is derived, i.e., partially or completely altered or removed from the natural state through human intervention. For example, proteins contained in the sample can be isolated according to standard methods, for example using lytic enzymes, chemical solutions, or isolated by protein-binding resins following the manufacturer's instructions.

The methods can include incubating the sample, e.g., 7.5 μl, 25 μl, 50 μl, 100 μl, 250 μl, 500 μl, 750 μl, 1 ml, 2 ml, 2.5 ml, or 10 ml of the sample, with a capture reagent (e.g., an antibody, nanobody, or antigen-binding fragment thereof as described herein). In some embodiments, the sample is diluted, e.g., 1:1, 1:2, 1:3, 1:4, 1:5, 1:6, 1:8, 1:10, or 1:20, and any ranges therebetween having the foregoing as endpoints, e.g., 1:1 to 1:20, or 1:1 or 1:10. In some embodiments, the sample is diluted with a buffer; an exemplary sample diluent buffer is described herein, and can comprise a detergent, e.g., Triton-X 100, Tween 20, NP-40, Brij35, Brij58, or C12E8, for example, present at about 0.05%-2% of the sample. “About” as used herein means plus or minus 10%.

In some embodiments, the sample is contacted with the capture reagent for a time sufficient for ORF1p present in the sample to bind to the capture reagent, e.g., for at least 5, 10, 15, 20, 30, or 45 minutes, or at least 1, 2, 3, 4, 5, or 6 hours, up to 1, 2, 3, 4, 5, 6, 8, 10, 12, 18, or 24 hours. In preferred embodiments, the capture reagent is on a bead, e.g., a paramagnetic bead. The capture reagent bound to the ORF1p is then incubated in the presence of detection antibodies (e.g., an antibody, nanobody, or antigen-binding fragment thereof as described herein), and the presence and/or quantity of bound antibodies is determined.

The presence and/or level of ORF1p protein can be evaluated using methods known in the art. In preferred embodiments, the methods include the use of highly sensitive or ultrasensitive and preferably multiplex detection methods including Meso Scale Discovery (MSD); Single-Molecule Arrays (SIMOA); droplet digital ELISA (ddELISA),²⁶ Molecular On-bead Signal Amplification for Individual Counting (MOSAIC),³⁰ Single-Molecule Counting (SMC); nucleic acid linked immune-sandwich assay (NULISA); Spear Bio's NAB-SURE (a cell-free assay that uses real-time PCR systems to quantify neutralizing antibodies (NAbs)); LUMINEX (immunoassay that precisely measures multiple analytes in one sample); SOMAscan Assays; mass spectrometry (e.g., MALDI-MS) and mass cytometry (e.g., CyTOF) (see, e.g., Cohen and Walt, Chem. Rev. 2019, 119, 293-321).

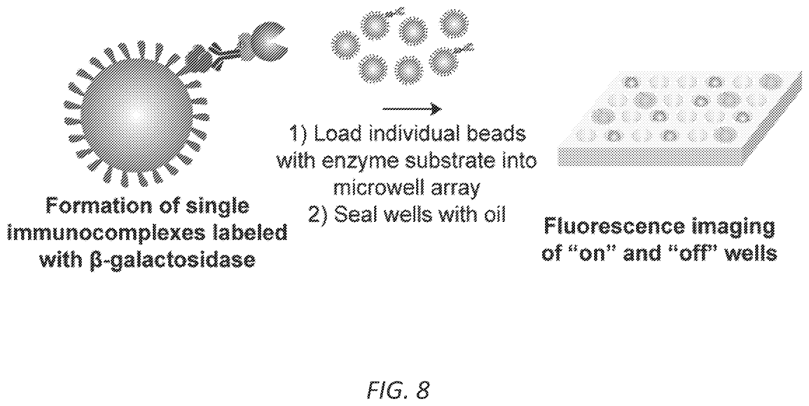

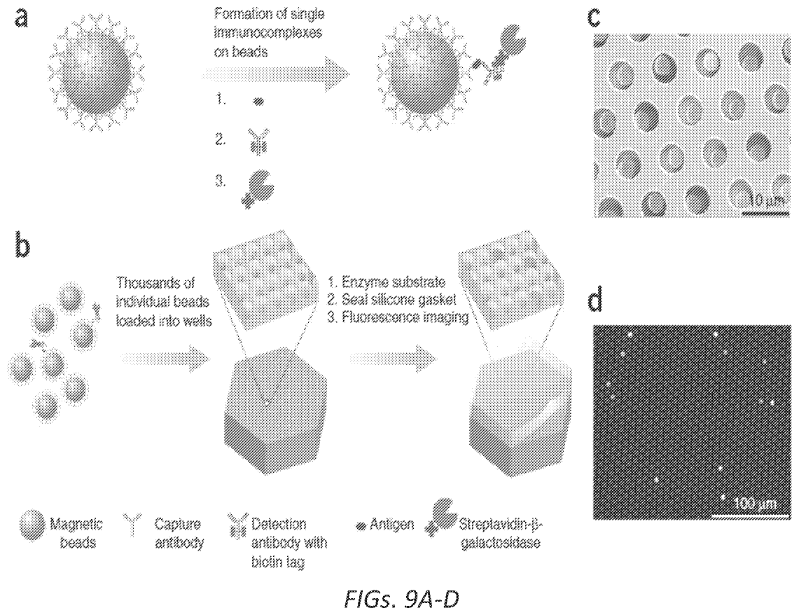

In some embodiments, the ORF1p protein in blood for cancer detection is measured using SIMOA or MOSAIC assays (11, 30, 39). SIMOA assays have several advantages over the conventional ELISA, the current gold standard for protein detection in blood. First, SIMOA is 1000× more sensitive than ELISA and allows for quantification of analytes present at low concentrations (11). SIMOA can detect protein concentrations as low as 10⁻¹⁹M compared to conventional ELISA's ability to detect only 10⁻¹²M. Second, due to the high sensitivity of SIMOA, the serum samples can be more dilute, which reduces non-specific binding that arises from matrix effects (40,41). Third, SIMOA has a wide dynamic range that spans four orders of magnitude in concentration, and thus a single assay can be used to detect both low and high abundance markers (42). In some embodiments, the SIMOA technique achieves this high sensitivity by digitally counting the number of molecules in a sample by labeling and physically isolating each immunocomplex into femtoliter-sized wells (FIGS. 8, 9A-D). These advantages provide for detection and quantification of blood biomarkers such as ORF1p for developing a robust assay.

In preferred embodiments, an ELISA method, e.g., SIMOA, MOSAIC, or another ultrasensitive method, is used; in preferred embodiments, the capture antibody is Ab54 (ab246320, AbCam) or Nb5, which is described further herein. In some embodiments, the detection antibody is Ab6 (ab246317, AbCam). In some embodiments, the capture/detection pair is a pair described herein, e.g., in Table 2 or 34H7/Ab6, 62H12/Ab6, 34H7/Nb5-5LL, 62H12/Nb5-5LL, 4H1/Nb5-5, or 4H1/Nb5-5LL.

In some embodiments, mass spectrometry, and particularly matrix-assisted laser desorption/ionization mass spectrometry (MALDI-MS) and surface-enhanced laser desorption/ionization mass spectrometry (SELDI-MS), are used for the detection of biomarkers. (See U.S. Pat. Nos. 5,118,937; 5,045,694; 5,719,060; 6,225,047).

In some embodiments, other methods can be used, e.g., standard electrophoretic and quantitative immunoassay methods for ORF1p proteins, including but not limited to, Western blot; enzyme linked immunosorbent assay (ELISA); Enzyme-Linked Immunospot (ELISPOT); biotin/avidin type assays; protein array detection, e.g., protein microarrays; radio-immunoassay; immunohistochemistry (IHC); immune-precipitation assay; flow cytometry/FACS (fluorescent activated cell sorting); Proximity Ligation Assay (PLA); lateral flow assay; surface plasmon resonance (SPR); optical imaging; Spear Bio's NAB-SURE (a cell-free assay that uses real-time PCR systems to quantify neutralizing antibodies (NAbs); and mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687; Cohen and Walt, Chem. Rev. 2019, 119, 293-321). The methods typically include revealing labels such as fluorescent, chemiluminescent, radioactive, and enzymatic or dye molecules that provide a signal either directly or indirectly, or oligo labels that can be used, e.g., in MOSAIC or other digital ELISA platforms, immuno-PCR. As used herein, the term “label” refers to the coupling (i.e., physical linkage) of a detectable substance, such as a radioactive agent or fluorophore (e.g., phycoerythrin (PE) or indocyanine (Cy5)), to an antibody or probe, as well as indirect labeling of the probe or antibody (e.g. horseradish peroxidase, HRP) by reactivity with a detectable substance.

The methods can also include comparing the presence and/or level with one or more references, e.g., a control reference that represents a normal level of ORF1p, e.g., a level in an unaffected subject, and/or a disease reference that represents a level of the proteins associated with cancer, e.g., a level in a subject having cancer. Suitable reference values can include undetectable levels of ORF1p or levels below e.g., 0.01, 0.005, or 0.001 pg/mL for subjects without cancer.

In some embodiments, the presence and/or level of the ORF1p is comparable to the presence and/or level of ORF1p in the disease reference, and the subject has one or more symptoms associated with cancer, then the subject has cancer. In some embodiments, the subject has no overt signs or symptoms of cancer, but the presence and/or level of one or more of the proteins evaluated is comparable to the presence and/or level of the protein(s) in the disease reference, then the subject has cancer or an increased risk of developing cancer. In some embodiments, once it has been determined that a person has cancer, or has an increased risk of developing cancer, then the subject can be selected or identified for further evaluation, e.g., using other blood-based diagnostics (e.g., biomarker panels), imaging, or biopsy to identify tumors or cancer, and/or a treatment, e.g., as known in the art or as described herein, can be selected and/or administered.

Suitable reference values can be determined using methods known in the art, e.g., using standard clinical trial methodology and statistical analysis. The reference values can have any relevant form. In some cases, the reference comprises a predetermined value for a meaningful level of ORF1p, e.g., a control reference level that represents a normal level of ORF1p, e.g., a level in an unaffected subject or a subject who is not at risk of developing a disease described herein, and/or a disease reference that represents a level of ORF1p associated with cancer, e.g., a level in a subject having cancer.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have cancer, does not have a risk of developing cancer, or does not later develop cancer.

A disease reference subject is one who has (or has an increased risk of developing) cancer. An increased risk is defined as a risk above the risk of subjects in the general population.

In some embodiments, the level of ORF1p in a subject being greater than or equal to the reference level of ORF1p is indicative of the presence or risk of developing cancer, and the level of ORF1p in a subject being less than or equal to a reference level of ORF1p is indicative of the absence of disease or normal risk of the disease.

Thus, in some embodiments, to assess whether a subject has cancer in the clinic, the method can include first log transforming the ORF1p values and then assigning a predicted probability, e.g., using a logistic regression model, to produce a probability score. If a subject has a predicted probability score above a selected threshold, e.g., at least 50%, the subject would be predicted to have cancer (e.g., assigned to a cancer category). If the predicted probability score is below the selected threshold, e.g., 50%, the subject would be predicted to be healthy (e.g., assigned to a healthy category).

In some embodiments, the level of ORF1p is used to calculate a score, e.g., along with one or more additional variable, e.g., age. The score can be calculated, e.g., using an algorithm such as summation, or weighted summation, of the (normalized) levels of the variables. Specific algorithms can be identified using known statistical methods including PCA, linear regression, SVM (support vector machine), decision tree, KNN (K-nearest neighbors), K-means, gradient boosting, or random forest methods.

For example, in some embodiments, an exemplary model uses a logistic regression analysis wherein each variable (X) gets a weight (B). In the exemplary equation below, the weights (B) are calculated for each marker, and there can be unique B values for each of the biomarkers.

$$Ln ( \frac{P}{1 - P} ) = {β}_{0} + {β}_{1} {X}_{1} + {β}_{2} {X}_{2} + … + {β}_{k} {X}_{k}$$

In the clinic, the measured ORF1p values (X values) can be used to obtain a probability score a patient has or will have cancer by plugging in the measured biomarker values (X) into the equation and then calculating a probability value (P). In some embodiments, the clinical procedure to obtain the individual's probability of having cancer would be as follows:

First, blood would be drawn from the screenee. Second, the screenee's blood concentration of ORF1p protein in the panel would be measured, e.g., using Simoa. Third, the screenee's predicted probability of having cancer would be calculated based on a logistic regression formula with a dependent variable of the natural log of [(probability of having cancer)/(probability of not having cancer)], and with independent variables of age and ORF1p. The predicted probability could then inform discussions between the screenee and physician as to how best to proceed, such as a decision that no further follow-up is necessary or to pursue confirmatory radiologic imaging.

In some embodiments, the amount by which the level (or score) in the subject is less than the reference level (or score) is sufficient to distinguish a subject from a control subject, and optionally is a statistically significantly less than the level (or score) in a control subject. In cases where the level (or score) of the biomarker(s) in a subject being equal to the reference level (or score) of the biomarker(s), the “being equal” refers to being approximately equal (e.g., not statistically different).

The predetermined value can depend upon the particular population of subjects (e.g., human subjects) selected. For example, an apparently healthy population will have a different ‘normal’ range of levels of the biomarker(s) than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

In some embodiments, a plurality of assays are performed with different combinations of antibodies as described herein, e.g., to improve sensitivity and/or specificity.

### Nanobodies

Described herein are nanobodies (also referred to as VHH antibodies), as well as antigen-binding domains thereof. The antibodies provided herein in one aspect comprise an antigen binding site in a single polypeptide. The antibodies are therefore herein referred to as “single domain antibodies”. Single domain antibodies are also known as nanobodies. The single antibodies disclosed herein may, though, in certain embodiment be bispecific or multispecific single domain antibodies as described elsewhere herein, where two single domain antibodies are coupled.

A single domain antibody is an antibody fragment consisting of a single monomeric variable antibody domain. Like a whole antibody, it is able to bind selectively to a specific antigen. Single domain antibodies typically have molecular weights in the range of 12-15 kDa, i.e. much lower than common antibodies, ranging typically from 150 to 160 kDa. Single domain antibodies are also smaller than Fab fragments (˜50 kDa) of heterotetrameric antibodies comprising one light chain and half a heavy chain.

In some embodiments, the antibodies used in the present methods are single domain antibodies, preferably derived from camelid antibodies, preferably llama antibodies, including functional homologs, fragments thereof and fusion macromolecules containing a VHH domain covalently linked to glycan, nucleic acid, protein, or chemical groups not being a macromolecule.

The single domain VHH antibodies described herein preferably comprise one or more CDRs from Table B, e.g., SEQ ID NO:1. In particular, the CDRs may identify the specificity of the antibody and accordingly it is preferred that the antigen binding site comprises one or more CDRs, preferably at least 1, more preferably at least 2, yet more preferably 3 or more CDRs. In one embodiment, the single domain antibody comprises 1 CDR. In one embodiment, the single domain antibody comprises 2 CDRs. In preferred embodiments, the single domain antibody comprises 3 CDRs and four framework regions. Methods for CDR swapping in VHH antibodies are known, see, e.g., Zupancic et al., Cell Chem Biol. 2021 Sep. 16; 28(9):1379-1388.e7; Saerens et al., J Mol Biol. 2005 Sep. 23; 352(3):597-607; Muyldermans et al., FEBS J. 2021 April; 288(7): 2084-2102.

In some embodiments, the nanobodies comprise a VHH sequence QVQLVESGGDLVQAGGSLRLSCAVSGGTSSNYGMGWFRQAPGKEREFVSSISW SGSRTLYSDSVKGRFTISRDNAKNTVDLQMNSLKPEDTAVYYCTAVREYRDYP QRDNFDYWGQGTQVTVS (SEQ ID NO:1). The bold sequences represent CDR1 (GGTSSNYG, SEQ ID NO:2), CDR2 (ISWSGSRT, SEQ ID NO:3), and CDR3 (TAVREYRDYPQRDNFDY, SEQ ID NO:4) (identified based on IMGT numbering).

In preferred embodiments, the nanobodies comprise a sequence that is at least 90, 95, 97, 99, or 100% identical to SEQ ID NO:1. In some embodiments, any mutations or substitutions are in a framework region (not in a CDR) and do not significantly affect binding to the target antigen (ORF1p).

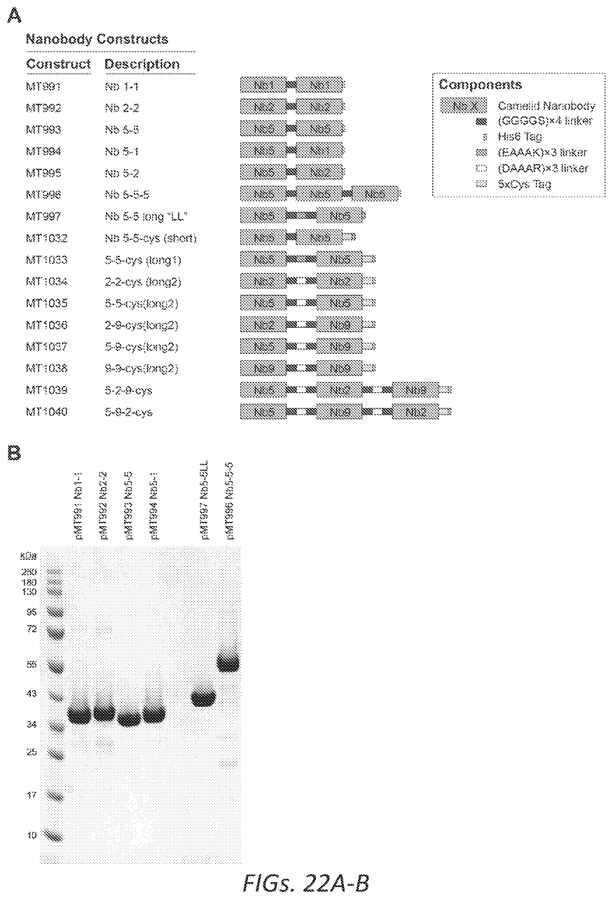

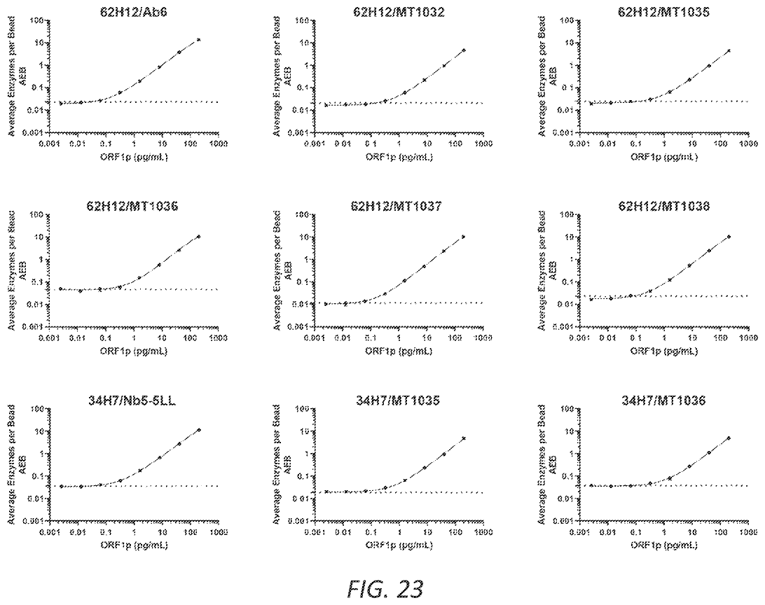

In some embodiments, concatemers of the nanobody sequences are used, e.g., wherein 2, 3, 4, 5 or more of the C5 nanobodies are fused, optionally with intervening linkers therebetween. Exemplary nanobody concatemers include MT1032, MT1033, MT1034, MT1035, MT1036, MT1037, MT1038, MT1039, and MT1040 (see FIG. 22A). A variety of suitable linkers are known to those of skill in the art and are not limited by any specific sequences disclosed herein. In some embodiments, the polypeptide linker is comprised of naturally, or non-naturally, occurring amino acids. In some embodiments, the linker comprises amino acids that allow for flexibility. In some embodiments, the linker comprises amino acids that allow for suitable solubility. In some embodiments, the linker comprises glycine amino acids. In some embodiments, the linker comprises glycine and serine amino acids. In certain embodiments, the linker comprises one or more sets of glycine/serine repeats. In some embodiments, the polypeptide linker is selected from the group consisting of: (GGGGS)n wherein n=1-4 (SEQ ID NO: 5), GGGGS (SEQ ID NO:6), GGGGSGGGGS (SEQ ID NO:7), GGGGSGGGGSGGGGS (SEQ ID NO:8), GGGGSGGGGSGGGGSGGGGS (SEQ ID NO:9 and (GGGGA)n wherein n=1-4 (SEQ ID NO:20), or and rigid helical linker, e.g., (EAAAK)n or (DAAAR)n, where n=1-4, preferably n=3, SEQ ID NO:11 and SEQ ID NO:12, respectively). In some embodiments, the linker comprises GGGGSGGGGSGGGGS (SEQ ID NO:8). In some embodiments, the linkers are preferably 5-100, 5-80, or 10-80 amino acids long, and comprise GGGGSGGGGSGGGGSGGGGS (SEQ ID NO:9) or GGGGSGGGGSGGGGSGGGGSEAAAKEAAAKEAAAKSGGGGSGGGGSGGGGSG GGGS (SEQ ID NO:13).

### Anti-ORF1p Monoclonal Antibodies

Described herein are monoclonal antibodies 62H12, 64C6, 33A8, 61A11, 36D12, 34H7, 50E9, 34C5, 55A6, and 42D10, and derivatives and antigen-binding fragments thereof. The term “antibody” refers to an immunoglobulin molecule or immunologically active portion thereof, i.e., an antigen-binding portion (e.g., Fv, Fab, Fab′, F(ab′)2 or other antigen-binding subsequences). In preferred embodiments, the antibodies comprise a sequence that is at least 90, 95, 97, 99, or 100% identical to a sequence set forth herein. In some embodiments, any mutations or substitutions are in a framework region (not in a CDR) and do not significantly affect binding to the target antigen (ORF1p).

The term “monoclonal antibody” as used herein refers to an antibody obtained from a population of substantially homogeneous antibodies, e.g., the individual antibodies comprising the population are identical except for possible naturally-occurring mutations that may be present in minor amounts. An antibody can be monoclonal. An antibody can be a human or humanized antibody. The term “monoclonal antibody” encompasses intact and full-length monoclonal antibodies as well as antibody fragments (e.g., Fab, Fab′, F(ab′)₂, Fv), single chain antibodies (e.g., scFv), fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region residues of the human heavy chain or light chain variable regions are replaced by corresponding residues from nonhuman antibody. Furthermore, humanized antibodies can comprise residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, CH2, CH3, Fc), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

“Antibody fragments” can include a portion of an intact antibody, preferably the antigen binding or variable region of the intact antibody. Examples of antibody fragments include Fab, Fab′, F(ab′)2, and Fv fragments; diabodies; linear antibodies; single-chain antibody molecules; and multispecific antibodies formed from antibody fragments. “Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. X-ray crystallography may be used to characterize an epitope on a target protein by analyzing the amino acid residue interactions of an antigen/antibody complex.

“Fv” includes the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgB1, IgG2, IgG3, IgG4, IgA, and IgA2. “Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding.

In various embodiments, the antibody or antigen binding fragment thereof comprises a human or humanized antibody. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. Methods for humanizing non-human antibodies are well known in the art.

The ORF1p antibodies described herein can be affinity matured, for example using selection and/or mutagenesis methods known in the art. In general, an “affinity matured” antibody is one with one or more alterations in one or more hyper variable regions thereof which result in an improvement in the affinity of the antibody for antigen, compared to a parent antibody which does not possess those alteration(s). In one embodiment, an affinity-matured antibody has nanomolar or even picomolar affinities for the target antigen. Preferred affinity matured antibodies have an affinity that is five times, more preferably 10 times, even more preferably 20 or 30 times greater than the starting antibody (generally murine, humanized or human) from which the matured antibody is prepared.

An antibody that “binds to,” “specifically binds to,” or is “specific for” a particular polypeptide or an epitope on a particular polypeptide is one that binds to that particular polypeptide or epitope on a particular polypeptide without substantially binding to any other polypeptide or polypeptide epitope. The term “specifically binds” as used herein refers to a ORF1p agent (e.g., an anti-ORF1p antibody) that interacts more frequently, more rapidly, with greater duration, with greater affinity, or with some combination of the above to a particular antigen, epitope, protein, or target molecule than with alternative substances. In some cases, the ORF1p antibody may or may not be cross reactive with ORF1p-related proteins (e.g., may have highest affinity for one, such as human ORF1p, and lower affinity for others, such as ORF2p.

In some embodiments, the antibody VH and VL domains described herein are fused to a constant region, e.g., as shown in Table A.

TABLE A
Sequences of Exemplary Constant Regions

| | | |
|---|---|---|
| Human | Glm (z) | ASTKGPSVFPLAPSSKSTSGGTAALGCLVKDYFPEPVTVSWN |
| IgG1 | allele | SGALTSGVHTFPAVLQSSGLYSLSSVVTVPSSSLGTQTYICN |
| Constant | (K214/ | VNHKPSNTKVDKKVEPKSCDKTHTCPPCPAPELLGGPSVFLF |
| (IGHG1) | D356/ | PPKPKDTLMISRTPEVTCVVVDVSHEDPEVKFNWYVDGVEVH |
| | L358) | NAKTKPREEQYNSTYRVVSVLTVLHQDWLNGKEYKCKVSNKA |
| | | LPAPIEKTISKAKGQPREPQVYTLPPSRDELTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPGK (SEQ ID |
| | | NO:14) |
| Human | G1m (z) | ASTKGPSVFPLAPSSKSTSGGTAALGCLVKDYFPEPVTVSWN |
| IgG1 | allele | SGALTSGVHTFPAVLOSSGLYSLSSVVTVPSSSLGTQTYICNVN |
| Constant | with | HKPSNTKVDKKVEPKSCDKTHTCPPCPAPEAAGAPSVFLFPPK |
| (IGHG1) | L234A/ | PKDTLMISRTPEVTCVVVDVSHEDPEVKFNWYVDGVEVHNAKT |
| LALAGA | L235A/ | KPREEQYNSTYRVVSVLTVLHQDWLNGKEYKCKVSNKALPAPI |
| | G237A | EKTISKAKGQPREPQVYTLPPSRDELTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK (SEQ ID NO: 15) |
| Human | WT | LGGPSVFLFPPKPKDTLMISRTPEVTCVVVDVSHEDPEVKENWYVDGVEV |
| IgG1 Fc | | HNAKTKPREEQYNSTYRVVSVLTVLHQDWLNGKEYKCKVSNKALPAPIEK |
| region | | TISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEALHN |
| | | HYTQKSLSLSPGK (SEQ ID NO:16) |
| Human | Uniprot | RTVAAPSVFIFPPSDEQLKSGTASVVCLLNNFYPREAKVQWK |
| IgK | P01834 | VDNALQSGNSQESVTEQDSKDSTYSLSSTLTLSKADYEKHKVY |
| Constant | | ACEVTHQGLSSPVTKSENRGEC (SEQ ID NO: 17) |
| (IGKC) | | |
| Mouse | Uniprot | AKTTPPSVYPLAPGSAAQTNSMVTLGCLVKGYFPEPVTVTWN |
| IgG1 | P01868 | SGSLSSGVHTFPAVLQSDLYTLSSSVTVPSSPRPSETVTCNV |
| Constant | | AHPASSTKVDKKIVPRDCGCKPCICTVPEVSSVFIFPPKPKD |
| (IGHG1 | | VLTITLTPKVTCVVVDISKDDPEVQFSWFVDDVEVHTAQTOP |
| Mouse) | | REEQFNSTFRSVSELPIMHQDWLNGKEFKCRVNSAAFPAPIE |
| | | KTISKTKGRPKAPQVYTIPPPKEQMAKDKVSLTCMITDFFPE |
| | | DITVEWQWNGQPAENYKNTQPIMNTNGSYFVYSKLNVOKSNW |
| | | EAGNTFTCSVLHEGLHNHHTEKSLSHSPGK (SEQ ID NO: 18) |
| Mouse | Uniprot | KTTPPSVYPLAPGCGDTTGSSVTLGCLVKGYFPESVTVTWNS |
| IgG2b | P01867 | GSLSSSVHTFPALLQSGLYTMSSSVTVPSSTWPSQTVTCSVA |
| Constant | | HPASSTTVDKKLEPSGPISTINPCPPCKECHKCPAPNLEGGP |
| (IGG2B | | SVFIFPPNIKDVLMISLTPKVTCVVVDVSEDDPDVQISWFVN |
| Mouse) | | NVEVHTAQTQTHREDYNSTIRVVSTLPIQHQDWMSGKEFKCK |
| | | VNNKDLPSPIERTISKIKGLVRAPQVYILPPPAEQLSRKDVS |
| | | LTCLVVGFNPGDISVEWTSNGHTEENYKDTAPVLDSDGSYFI |
| | | YSKLNMKTSKWEKTDSFSCNVRHEGLKNYYLKKTISRSPGLD |
| | | LDDICAEAKDGELDGLWTTITIFISLFLLSVCYSASVTLFKV |
| | | KWIFSSVVELKQKISPDYRNMIGQGA (SEQ ID NO:19) |
| Mouse | Uniprot | RADAAPTVSI FPPSSEQLTSGGASVVCFLNNFYPKDINVKWK |
| IgK | P01837 | IDGSERQNGVLNSWTDQDSKDSTYSMS STLTLTKDEYERHNSYT |
| Constant | | CEATHKTSTSPIVKSFNRNEC (SEQ ID NO:20) |
| (IGKC | | |
| Mouse) | | |

### Identity

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60, at least about 60-80 nucleotides or amino acids in length, or any integral value there between. In some embodiments, identity exists over a longer region than 60-80 nucleotides or amino acids, such as at least about 80-100 nucleotides or amino acids, and in some embodiments the sequences are substantially identical over the full length of the sequences being compared, for example, (i) the coding region of a nucleotide sequence or (ii) an amino acid sequence.

The phrase “conservative amino acid substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of an alanine for a valine is considered to be a conservative substitution. Methods of identifying nucleotide and amino acid conservative substitutions that do not eliminate binding are well-known in the art.

In some embodiments, the nanobodies or antibodies can be conjugated with another protein or peptide, e.g., to form multifunctional protein/peptides. Exemplary conjugates can include Fc fragments. See, e.g., Bao et al., EJNMVII Res. 2021; 11: 6; Hoey et al., Exp Biol Med (Maywood). 2019 December; 244(17): 1568-1576; Bever et al., Anal Bioanal Chem. 2016 September; 408(22): 5985-6002.

In some embodiments, the nanobodies or antibodies can be conjugated to or include a tag, e.g., a label (detectable moiety) or a purification moiety, e.g., FLAG, hexahistidine (6-HIS), or hemagglutinin (HA). Examples of detectable substances for use as labels include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, or ³H.

Also provided herein are kits and compositions comprising the nanobodies and/or antibodies as described herein, as well as nucleic acids encoding the nanobodies and/or antibodies, vectors comprising the nucleic acids (e.g., viral vectors or plasmids, preferably comprising regulatory sequences such as promoters to drive expression of the nanobodies and/or antibodies), and host cells (e.g., bacterial, yeast, insect or mammalian cells) comprising the nucleic acids and optionally expressing the nanobodies and/or antibodies.

### Methods of Treatment, Screening, and Monitoring Efficacy of Treatment

As shown herein, plasma ORF1p levels determined at the time of diagnosis are prognostic of overall survival in cancer, including in colorectal and gastroesophageal cancers, and can be used to monitor treatment response over time. This application could allow patients to be stratified into high and low risk group to receive additional treatment, such as chemotherapy, more aggressive chemotherapy, or additional surgery, especially in colorectal, breast, or prostate cancers, where multiple treatments are available. Thus the methods described herein include methods for the treatment of cancer. Generally, the methods include selecting and optionally administering a therapeutically effective amount of a treatment for cancer to a subject who has been determined to be in need of such treatment by a method described herein. Treatments for cancer can depend on the type of cancer, and can include radiation, surgical resection, chemotherapy, hormone/endocrine therapy, and/or immunotherapy. In some embodiments, the cancer is a carcinoma, e.g., ovarian, breast, liver, colon/colorectal, lung, esophageal, prostate, gastric, head and neck, brain, soft tissue, kidney, gallbladder, bile duct (cholangiocarcinoma), bladder, uterine, or pancreatic cancer. In some embodiments, the cancer is of origin in blood, bone marrow, brain, skin, or soft tissue in origin, especially lymphoma, leukemia, myeloma, glioblastoma, or melanoma.

In some embodiments, where a subject is identified as likely to have ovarian cancer, the subject is treated with surgical resection and optionally with chemotherapy and/or immunotherapy. Chemotherapy can include, e.g., paclitaxel and carboplatin, docetaxel and carboplatin, or carboplatin and pegylated liposomal doxorubicin, gemcitabine, toptecan, etoposide, and/or bevacizumab; PARP inhibitors, e.g., olaparib; or hormonal therapy, e.g., tamoxifen or letrozole.

The methods can also include sending the subject for additional screening such as referral to additional workups e.g., trans-vaginal sonography, uterine lavage, or falloposcopy, based on an updated posterior probability of having ovarian cancer, optionally combining ORF1p results with other clinical features and potentially other biomarkers (e.g., CA125 and/or HE4 for ovarian cancer).

The methods can also be used for monitoring response to a treatment, e.g., to radiation, surgical resection, chemotherapy, hormone/endocrine therapy, and/or immunotherapy. The methods can include determining a baseline level of ORF1p in the subject using a method described herein; administering a treatment, e.g., one or more doses of a treatment, and determining a subsequent level of ORF1p in the subject, e.g., an on-treatment (when obtained while the treatment is ongoing) and/or post-treatment (when obtained after the treatment is completed. A decrease in the level of ORF1p in the subject from the baseline to the subsequent level indicates that the subject is responding or has responded to the therapy. The methods can also be used to monitor a subject who is in remission, to determine whether the subject remains in remission (e.g., has levels of ORF1p that are at or below a threshold, e.g., the level of detection in a sample, or a level in a subject who does not have cancer). If a treatment is effective, the methods can include continuing the treatment. If a treatment is not effective (e.g., the level of ORF1p does not decrease, or increases), the methods can include selecting and optionally administering a different treatment.

### Kits and Assay Reagents

Also provided herein are kits and assay reagents comprising the nanobodies and antibodies (including antigen binding fragments thereof) described herein. In some embodiments, the kits or reagents comprise solid surfaces onto which the nanobodies or antibodies are coupled. In some embodiments, the surfaces are beads, e.g., paramagnetic or polymeric beads.

### EXAMPLES

The invention is further described in the following examples, which do not limit the scope of the invention described in the claims.

### Materials and Methods

The following materials and methods were used in the Examples below.

Materials. All affinity reagents used in this work are listed in Table E. Conjugation reagents, paramagnetic beads, and assay buffers were obtained from Quanterix Corporation. DNA oligos used in the MOSAIC assay were obtained from Integrated DNA Technologies. Antibodies used in final Simoa and MOSAIC assays (monoclonals Ab6, Ab54, 62H12, 34H7) were additionally validated by Western blotting.

TABLE E
Affinity reagents used across all screening experiments.

| Affinity reagent | Type | Source |
|---|---|---|
| Ab6 | Rabbit monoclonal antibody | Abcam (ab246317) |
| Ab54 | Rabbit monoclonal antibody | Abcam (ab246320) |
| Nb5 | Nanobody | Rockefeller |
| | | University |
| 4H1 | Mouse monoclonal antibody | MilliporeSigma |
| | | (MABC1152) |
| JH73 | Rabbit monoclonal antibody | |
| JH74 | Rabbit monoclonal antibody | |
| D3W9O | Rabbit monoclonal antibody | Cell Signaling |
| | | Technology (88701) |
| Nb5-5 pMT993 | Homodimeric nanobody (short linker) | This study |
| Nb5-5LL pMT997 | Homodimeric nanobody (long linker) | This study |
| Nb1-1 pMT991 | Homodimeric nanobody (short linker) | This study |
| Nb2-2 pMT992 | Homodimeric nanobody (short linker) | This study |
| Nb5-1 pMT994 | Heterodimeric nanobody (short linker) | This study |
| Nb5-2 pMT995 | Heterodimeric nanobody (short linker) | This study |
| 33A8 | Rabbit monoclonal antibody | This study |
| 34C5 | Rabbit monoclonal antibody | This study |
| 34H7 | Rabbit monoclonal antibody | This study |
| 36D12 | Rabbit monoclonal antibody | This study |
| 42D10 | Rabbit monoclonal antibody | This study |
| 50E9 | Rabbit monoclonal antibody | This study |
| 55A6 | Rabbit monoclonal antibody | This study |
| 64C6 | Rabbit monoclonal antibody | This study |
| 61A11 | Rabbit monoclonal antibody | This study |
| 62H12 | Rabbit monoclonal antibody | This study |

Preparation of capture and detector reagents. All capture antibodies and nanobodies were obtained in or dialyzed into phosphate buffered saline (PBS). For the first-generation Simoa assay, 7×10⁸ carboxylated paramagnetic 2.7-μm beads (Homebrew Singleplex Beads, Quanterix Corp.) were first washed three times with 400 μL Bead Wash Buffer (Quanterix Corp.) and two times with 400 μL cold Bead Conjugation Buffer (Quanterix Corp.) before being resuspended in 390 μL cold Bead Conjugation Buffer. A 1 mg vial of 1-ethyl-3-(3-dimethylaminopropyl) carbodiimide hydrochloride (EDC) (Thermo Fisher Scientific) was then dissolved to 10 mg/mL in cold Bead Conjugation Buffer, and 10 μL was added to the beads. The beads were shaken for 30 minutes at 4° C. to activate the carboxyl groups on the beads, which were then washed once with 400 μL cold Bead Conjugation Buffer and resuspended in the capture nanobody solution (10 μg nanobody total), diluted in Bead Conjugation Buffer to a final volume of 400 μL. The beads were shaken for two hours at 4° C., washed twice with 400 μL Bead Wash Buffer, and resuspended in 400 μL Bead Blocking Buffer (Quanterix Corp.) before shaking at room temperature for 30 minutes to block the beads. After one wash each with 400 μL Bead Wash Buffer and Bead Diluent (Quanterix Corp.), the beads were resuspended in Bead Diluent and stored at 4° C. Beads were counted with a Beckman Counter Z Series Particle Counter before using in assays. For second-generation Simoa assays, the following bead coupling conditions were used: 4.2×10⁸ starting beads, 300 μL wash volumes, 6 μL EDC, and 40 μg antibody.

For biotinylation of detector antibodies or nanobodies, a 1 mg vial of Sulfo-NHS-LC-LC-biotin was freshly dissolved in 150 μL water and added at 80-fold molar excess to a 1 mg/mL solution of antibody or nanobody. The reaction mixture was incubated at 30 minutes at room temperature and subsequently purified with an Amicon Ultra-0.5 mL centrifugal filter (50K and 10K cutoffs for antibody and dimeric nanobody, respectively). Five centrifugation cycles of 14,000×g for five minutes were performed, with addition of 450 μL PBS each cycle. The purified biotinylated detector reagent was recovered by inverting the filter into a new tube and centrifuging at 1000×g for two minutes. Concentration was quantified using a NanoDrop spectrophotometer.

Recombinant ORF1p protein production. ORF1p was prepared as described (25); briefly, codon optimized human ORF1p corresponding to L1RP (L1 insertion in X-linked retinitis pigmentosa locus, GenBank AF148856.1) with N-terminal His6-TEV was expressed in E. Coli, purified by Ni-NTA affinity, eluted, tag cleaved in the presence of RNaseA, and polished by size exclusion in a buffer containing 50 mM HEPES pH 7.8, 500 mM NaCl, 10 mM MgCl2, and 0.5 mM tris(2-carboxyethyl) phosphine (TCEP), resulting in monodisperse trimeric ORF1p bearing an N-terminal glycine scar.

Nanobody generation and screening. Nanobodies were generated essentially as described (49, 52) using mass spectrometry/lymphocyte cDNA sequencing to identify antigen-specific nanobody candidates. Briefly, a llama was immunized with monodisperse ORF1p, and serum and bone marrow were isolated. The heavy chain only IgG fraction (VHH) was isolated from serum and bound to a column of immobilized ORF1p. Bound protein was eluted in SDS and sequenced by mass spectrometry, utilizing a library derived from sequencing VHH fragments PCR-amplified from bone marrow-derived plasma cells. Candidate sequences were cloned into an E. coli expression vector with C-terminal His6 tag and expressed in 50 ml cultures in E. coli Arctic Express RP (Agilent) with 0.2 mM IPTG induction at 12° C. overnight. Periplasmic extract was generated as follows: pellets were resuspended in 10 ml per L culture TES buffer (200 mM Tris-HCl, pH 8.0, 0.5 mM EDTA, and 500 mM sucrose), 20 ml/L hypotonic lysis buffer added (TES buffer diluted 1:4 with ddH2O), supplemented with 1 mM PMSF, 3 μg/ml Pepstatin A, incubated 45 min at 4° C., and centrifuged at 25,000×g for 30 min. The supernatant (periplasmic extract) was bound to ORF1p-conjugated Sepharose, washed 3 times, eluted with SDS at 70° C. for 10 min, and periplasmic extract and elution were analyzed by SDS-PAGE to assay expression and yield. ORF1p-binding candidates were purified as below and analyzed by ELISA.

Nanobody and multimeric nanobody purification. C-terminally His6-tagged nanobody constructs were expressed and purified essentially as described (49). Briefly, protein was expressed in E. coli Arctic Express RP (Agilent) with 0.2 mM IPTG induction at 12° C. overnight. Periplasmic extract (generated as above) was supplemented with 5 mM MgC₂, 500 mM NaCl, and 20 mM imidazole, purified by Ni-NTA chromatography, dialyzed into 150 mM NaCl, 10 mM HEPES, pH 7.4, and concentrated to 1-3 mg/ml by ultrafiltration. “5×Cys tail” constructs were purified with the addition of 5 mM TCEP-HCl in resuspension, wash, elution, and dialysis buffers.

Surface plasmon resonance (SPR) assays. Binding kinetics (kₐ, k_{d}, and K_{D}) of antibody and nanobody constructs for ORF1p were obtained on a Biacore 8K instrument (Cytiva). Recombinant ORF1p was immobilized on a Series S CM5 sensor chip at 1.5 μg/ml using EDC/NHS coupling chemistry according to the manufacturer's guidelines. Nanobodies and antibodies were prepared as analytes and run in buffer containing 20 mM HEPES pH 7.4, 150 mM NaCl, and 0.05% Tween-20. Analytes were injected at 30 μl/min in single-cycle kinetics experiments at concentrations of 0.1, 0.3, 1, 3.3, and 10 nM, with association times of 120-180 sec, and a dissociation time of 1200-7200 sec, depending on observed off-rate. Residual bound protein was removed between experiments using 10 mM glycine-HCl pH 3.0. Data were analyzed using Biacore software, fitting a Langmuir 1:1 binding model to sensorgrams to calculate kinetic parameters.

For epitope binning, pairs of antibodies were sequentially flowed over immobilized ORF1p using Biacore tandem dual injections according to the manufacturer's guidelines. Antibodies were injected at concentrations of 200 nM with a flow rate of 10 μl/min. Contact time for the first antibody was 120 sec, followed by 150 sec for the second antibody, then a 30 sec dissociation. Response signal for the second antibody was measured in a 10 sec window at the beginning of dissociation. The chip was regenerated between experiments with glycine pH 3.0 as above. Data were analyzed using the Biacore software epitope binning module.

ORF1p Simoa assays. Simoa assays were performed on an HD-X Analyzer (Quanterix Corp.), with all assay reagents and consumables loaded onto the instrument according to the manufacturer's instructions. 250,000 capture beads and 250,000 helper (non-conjugated) beads were used in each Simoa assay. A three-step assay configuration was used for the first- and second-generation assays, consisting of a 15-minute target capture step (incubation of capture beads with 100 μL sample), 5-minute incubation with detector reagent (0.3 μg/mL for both first- and second-generation assays), and 5-minute incubation with streptavidin-β-galactosidase (150 pM for first-generation assay; 300 pM for second-generation assays). The beads were washed with System Wash Buffer 1 (Quanterix Corp.) after each assay step. Upon the final wash cycle, the beads were loaded together with the fluorogenic enzyme substrate resorufin β-D-galactopyranoside into a 216,000-microwell array, which was subsequently sealed with oil. Automated imaging and counting of “on” and “off” wells and calculation of average enzyme per bead (AEB) were performed by the instrument. Calibration curves were fit using a 4PL fit with a 1/y² weighting factor, and the limit of detection (LOD) was determined as three standard deviations above the blank.

All plasma and serum samples were diluted four-fold in Homebrew Sample Diluent (Quanterix Corp.) with 1× Halt Protease Inhibitor Cocktail (ThermoFisher), with an additional 1% Triton-X 100 added in the second-generation assays. All recombinant ORF1p calibrators were run in triplicates, with four replicates for the blank calibrator, and all plasma and serum samples were run in duplicates. The average LOD across all sample runs was determined for each assay and depicted in each figure.

Healthy individual plasma and serum samples were obtained from the Mass General Brigham Biobank, with additional samples from the Penn Ovarian Cancer Research Center and Tomas Mustelin (University of Washington).

ORF1p large-volume MOSAIC assays. MOSAIC assays were performed as previously described, using 2 ml microcentrifuge tubes for the initial capture step. For each sample, 500 μL plasma was diluted four-fold in Homebrew Sample Diluent with protease inhibitor and 1% Triton-X 100 to a total volume of 2 mL. Briefly, 100,000 capture beads were incubated with sample and mixed for two hours at room temperature, followed by magnetic separation and resuspended in 250 μL System Wash Buffer 1 before transferring to a 96-well plate. The beads were then washed with System Wash Buffer 1 using a Biotek 405 TS Microplate Washer before adding 100 μL nanobody detector reagent (0.3 μg/mL, diluted in Homebrew Sample Diluent) and shaking the plate for 10 minutes at room temperature. After washing with the microplate washer, the beads were incubated with 100 μL streptavidin-DNA (100 pM, diluted in Homebrew Sample Diluent with 5 mM EDTA and 0.02 mg/mL heparin) with shaking for 10 minutes at room temperature, followed by another washing step. The beads were transferred to a new 96-well plate, manually washed with 180 μL System Wash Buffer 1, and resuspended in 50 μL reaction mixture for rolling circle amplification (RCA). The RCA reaction mixture consisted of 0.33 U/uL phi29 polymerase, 1 nM ATTO647N-labeled DNA probe, 0.5 mM deoxyribonucleotide mix, 0.2 mg/mL bovine serum albumin, and 0.1% Tween-20 in 50 mM Tris-HCl (pH 7.5), 10 mM (NH₄)₂SO₄, and 10 mM MgCl₂. The beads were shaken at 37° C. for one hour, followed by addition of 160 μL PBS with 5 mM EDTA and 0.1% Tween-20. After washing the beads once with 200 μL of the same buffer, the beads were resuspended in 140 μL buffer with 0.2% BSA. All samples were analyzed using a NovoCyte flow cytometer (Agilent) equipped with three lasers. Analysis of average molecule per bead (AMB) values was performed as previously described using FlowJo software (BD Biosciences) and Python. All code used for MOSAIC data analysis can be downloaded as part of the waltlabtools.mosaic Python module, which is available at github.com/tylerdougan/waltlabtools.

Targeted proteomics analysis of immunoprecipitated ORF1p. Protein levels the LINE-1 ORF1p (UniProt ID: Q9UN81) were determined with targeted proteomics using isotopically-labeled standard peptides (AQUA QuantProHeavy peptides with ¹³C¹⁵N-labeled C-terminal lysine or arginine, Thermo Fisher) for accurate quantification. Assays were developed for two quantotypic peptides of ORF1p, namely LSFISEGEIK and cysteine-alkylated NLEECITR (the approach is similar to the assay development described previously for other proteins (53)). Briefly, 3-6 mL patient plasma was diluted with an equal volume of 2× dilution buffer (PBS containing 2% Triton X-100, 10 mM EDTA, and 1 Pierce protease inhibitor tablet per 25 ml (2× concentration, Thermo) for a final concentration of 1% Triton X-100, 5 mM EDTA, and 1× protease inhibitor and bound to 7 million 62H12-conjugated magnetic beads for 1 hour at room temperature. Beads were washed 3 times with 5×PBS containing 0.1% tween 20 and 1× protease inhibitor, then once with the same buffer lacking tween 20, and eluted in 50 μl buffer containing 2% SDS and 50 mM Tris pH 8.5 by heating for 5 minutes at 95° C. with agitation. Separated eluates were subjected to in-gel digestion using trypsin (150 ng sequencing grade modified trypsin V5111; Promega) after reduction with 10 mmol/L dithiothreitol and alkylation with 55 mmol/L iodoacetamide proteins, prior to LC-MS analyses of the target peptides (53).

Classification models. Classification models were trained for (1) all healthy and all ovarian cancer patients measured by the second-generation assays; and (2) the subset of 51 ovarian cancer and 50 age-matched healthy female patients, obtained from Ronny Drapkin (University of Pennsylvania). Each dataset contained no missing values, and the measurements in the datasets were log-transformed and normalized beforehand for classification analysis of healthy and ovarian cancer subjects. Logistic regression was used for the univariate classifier and the k-nearest neighbors (KNN) and light gradient-boosting machine (LightGBM), which had the best performances among the classifiers, were used for the multivariate classifier, and implemented in Python 3.7.15 with scikit-learn version 1.0.2 package. Each classifier was given a weight optimization between classes to deal with data imbalance between healthy and cancer subjects, as well as hyperparameter tuning using grid search.

The performance of each biomarker in differentiating ovarian cancer subjects from healthy subjects was evaluated with fivefold cross validation by calculating accuracy, precision, recall, f1-value, sensitivity, specificity, and area under the receiver-operating characteristic (ROC) curve (AUC). A stratified five-fold cross-validation strategy randomly splits the positive and negative samples into five equally sized subsets. One positive subset and one negative subset were selected as the test dataset each time, and the other samples were used to train a classification model.

In the multivariate analysis, the Variance Inflation Factor (VIF) for the biomarkers was calculated, and any biomarkers with extremely high correlation with VIF greater than 10 were excluded from the classification model in advance.

Barrett's esophagus cases. A cohort of 75 esophageal biopsies with BE and varying degrees of dysplasia were assembled. Negative cases were screened to have no prior history of dysplasia. The mean age of the cohort was 67 years with a male predominance (M:F ratio=3.7:1). All samples were re-analyzed for histological features of dysplasia by three experienced gastrointestinal pathologists (LRZ, VD, OHY) who were blinded to the original diagnosis. A consensus was reached for 72 cases and the consensus diagnosis was used as the gold standard. There was moderate agreement between pathologists (kappa 0.43-0.51).

Colon cancer tissue microarray. 178 sequential CRCs resected by a single surgeon from 2011-2013 were assembled on a 3 mm core tissue microarray. All cases were independently scored by two pathologists. The mean age of the cohort was 65 years with 49.8% males. Mean follow-up was 25 months. At resection, 23% were stage I, 33% were stage II, 44% were stage III, and 1% were stage IV.

Ovarian Cancer Samples. Age-matched ovarian cancer (n=53) and healthy control (n=50) patient plasma samples were from University of Pennsylvania Ovarian Cancer Research Center, OCRC Tumor BioTrust Collection, Research Resource Identifier (RRID): SCR_02287.

Gastroesophageal cancer treatment cohort. Nineteen patients received systemic therapy, 3 of which also underwent surgical resection. Patients were treated with concurrent chemotherapy (carboplatin/taxol) and radiation (N=3), fluorouracil/leucovorin/oxaliplatin/docetaxel (FLOT, N=2), fluorouracil/leucovorin/irinotecan/oxaliplatin (FOLFIRINOX, N=2), fluorouracil/leucovorin/oxaliplatin (FOLFOX, N=9), FOLFOX+trastuzumab (N=1), pembrolizumab (N=1) or FOLFOX then chemoradiation (1). The mean age of the cohort was 76 years. All patients were male (100%). Fifty-eight percent had locally advanced disease (stage II-III) and 42% had advanced disease (stage IV) at the time of initial diagnosis. Sixty-eight percent (N=13) were deemed Responders to therapy while 32% (N=6) were deemed Non-Responders to standard therapy on review of re-staging imaging (CT and/or PET-CT) by investigators blinded to the assay results. Note that the on/post-treatment blood draw measured by Simoa often preceded these imaging studies.

Patient Consent. All plasma samples were obtained with informed written consent under IRB approved protocols at Mass General Brigham (MGB), University of Pennsylvania, and University of Washington. All experiments with patient samples were conducted under IRB approval and in accordance with ethical guidelines in the Belmont Report. Tissue samples were obtained with consent, or, where appropriate, with waiver of consent under MGB approved protocols.

Histochemistry: ORF1p immunohistochemistry was performed essentially as described using anti-ORF1 4H1 (Millipore)(8) diluted 1:3000 and re-optimized on a Leica Bond system (17). Cases were scored by three experienced gastrointestinal pathologists (MST, VD, OHY) at two institutions. LINE-1 in situ hybridization was performed as described using RNAscope catalog 565098 (Advanced Cell Diagnostics) on a Leica Bond system (17). The probe is complementary to the 5′ end of L1RP (L1 insertion in X-linked retinitis pigmentosa locus). Cases were scored by three experienced gastrointestinal pathologists (MST, VD, OHY).

Survival Analysis: Kaplan-Meier (KM) curves (54) were computed to study the association between overall survival and plasma ORF1p concentration in ovarian cancer, colorectal cancer and esophageal cancer. To investigate the association with survival, we classified ORF1p concentrations in two different ways. First, by classifying each of the three assays as positive if the signal was above the limit of detection (LoD)—in at least two out of three assays (majority vote method). Second, we evaluated whether ORF1p concentration measured by the most sensitive assay (62H12::Ab6) alone was associated with survival, classifying patients as ORF1p High and Low based on the cohort-specific median. The time variable was defined as days after diagnosis (GE and CRC) or treatment start (ovarian). Living patients were censored at the date of last assessment. Because age at diagnosis was significantly associated with poor prognosis in CRC and male sex was significantly associated with a poor prognosis in GE cancer, we applied a Cox proportional hazards regression model (55); ORF1p was found to be independently prognostic. Survival objects and KM curves were computed using the survival, ggpubr and survminer packages in R. All tests were performed using R version 4.3.1 (The R Project for Statistical Computing, R-project.org/). The proportional hazard assumption was tested by plotting the Schoenfeld residuals and applying the Grambsch-Therneau test using the ggcoxdiagnostics function in R. The effect of influential observations was assessed by plotting the Deviance residuals using the ggcoxdiagnostics function in R. Original data for survival are provided in a file “Supplementary Original Survival Data”.

### Example 1. Ultrasensitive Detection of Circulating ORF1P

Despite the elevated expression of ORF1p in tumor tissue in ovarian and other cancers, the levels of ORF1p and many other biomarkers shed from tumors are diluted into the bloodstream to very low levels. These circulating concentrations can exist well below the detection limits of conventional enzyme-linked immunosorbent assay (ELISA) and mass spectrometry methods, therefore necessitating ultrasensitive detection methods. Single Molecule Arrays (Simoa), a digital ELISA technology, enable 1000-fold increases in analytical sensitivity over conventional ELISA (FIG. 2B)¹¹. Utilizing Simoa technology, we recently reported an ultrasensitive digital assay to detect ORF1p in the blood²⁶.

The assay was optimized and expanded to detect concentrations of circulating ORF1p as low as sub-pg/mL (low femtomolar) in ovarian and other cancer patients, with exceptionally high specificity for cancer detection (FIG. 2C). Briefly, affinity reagents were screened as capture and detector pairs on the Simoa platform in a combinatorial fashion. Capture affinity reagents were conjugated to 2.7-μm paramagnetic beads via EDC chemistry, while detector affinity reagents were biotinylated using a SulfoNHS-LC-LC-biotin reagent. For each pair, a few concentrations of recombinant human ORF1p protein and a buffer blank were measured to determine signal-to-background. Select pairs were used to measure ORF1p levels in breast cancer cell lysate, and subsequently in breast cancer and healthy patient serum samples.

The reagents tested included 4H1 (Mouse monoclonal antibody targeting amino acids 35 to 44 of human ORF1p, MABC1152, Sigma-Aldrich), JH73 (Rabbit monoclonal antibody raised against the C-terminus of human ORF1p, Taylor et al., Cell. 2013 Nov. 21; 155(5):1034-48), and JH74 (Rabbit monoclonal antibody raised against the C-terminus of human ORF1p, Doucet-O'Hare et al., Proc Natl Acad Sci USA. 2015 Sep. 1; 112(35):E4894-900), see, e.g., Mita et al., eLife. 2018; 7: e30058; commercial antibodies Ab6 (ab246317, AbCam), Ab54 (ab246320, AbCam), D3W90 (#88701, Cell Signaling Technology); and 168006 (NovoPro Labs); Nanobody5 (Nb5), a nanobody described herein), and monoclonal antibodies 62H12, 64C6, 33A8, 61A11, 36D12, 34H7, 50E9, 34C5, 55A6, and 42D10 as described herein.

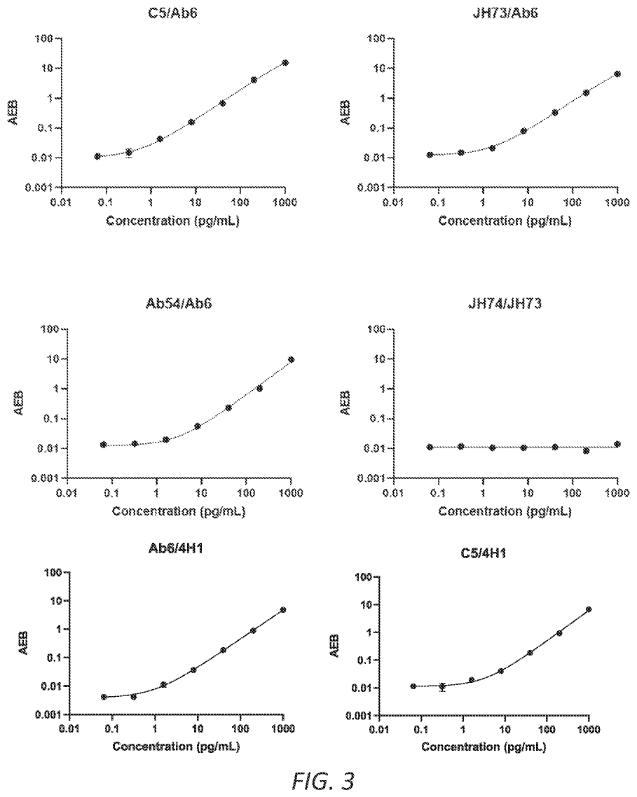

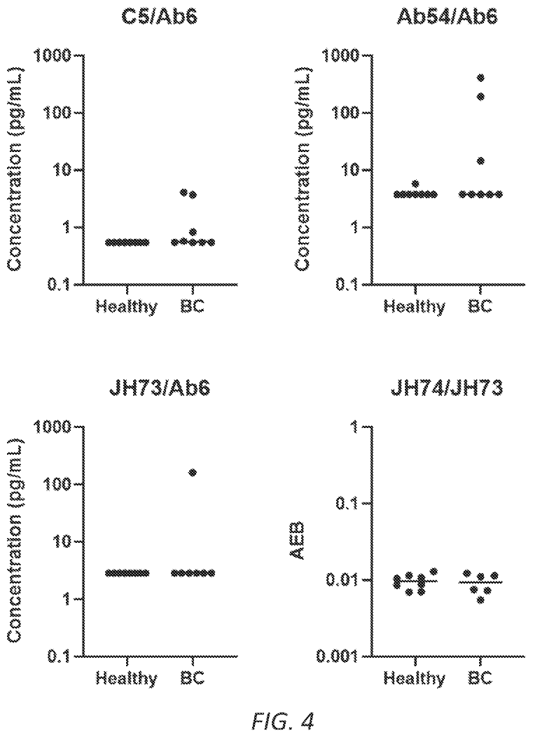

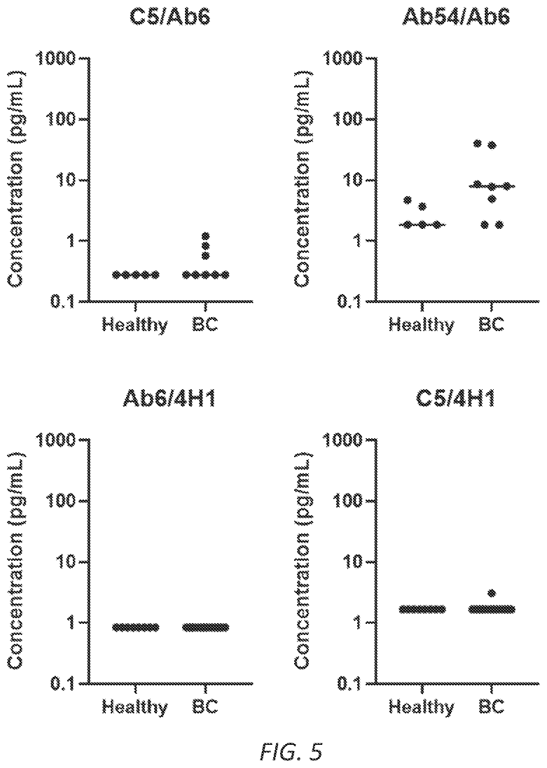

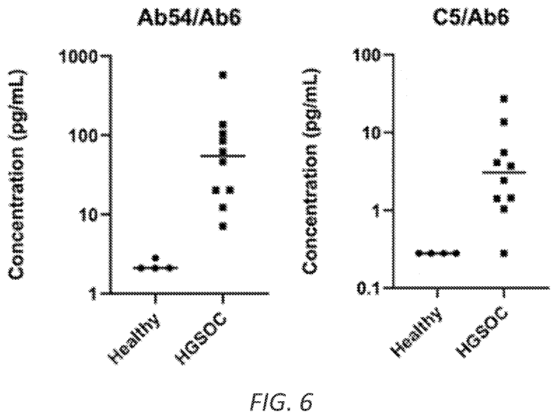

Signal to background ratio for the various pairs on the Simoa platform is shown in FIG. 2. These values were determined using recombinant human ORF1p protein and diluted breast cancer cell lysate, and the affinity reagent pairs with the highest signal to background ratios were chosen for further screening in a trial cohort of healthy and breast cancer patient serum samples. Representative calibration curves for select affinity reagent pairs are shown in FIG. 3, where the measured signal is average enzyme per bead (AEB). The measured ORF1p levels using select affinity reagent pairs on the Simoa platform in healthy and breast cancer patient serum samples are shown in FIGS. 4-5. The measured ORF1p levels using the Nb5 nanobody/Ab6 and Ab54/Ab6 capture/detector pairs in high-grade serous ovarian cancer and healthy patient plasma samples are shown in FIG. 6.

A final nanobody/antibody pair of Nb5 nanobody/Ab6 was selected due to its high specificity in healthy patients (undetectable baseline levels) and good sensitivity in cancer patients.

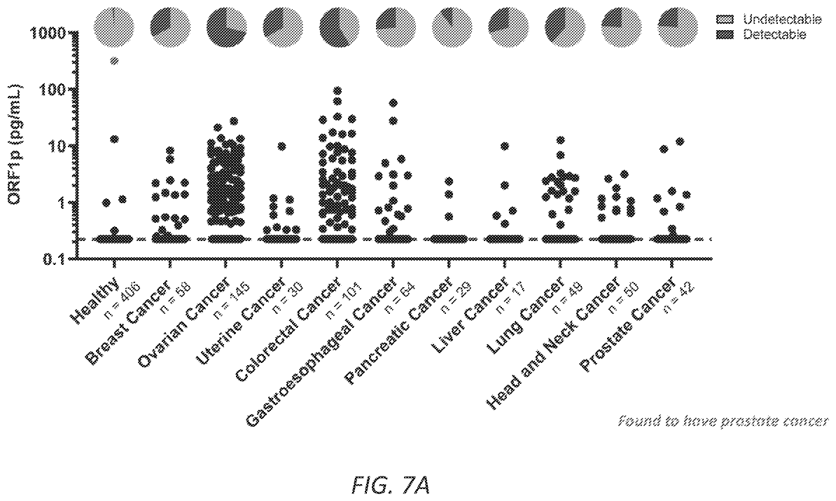

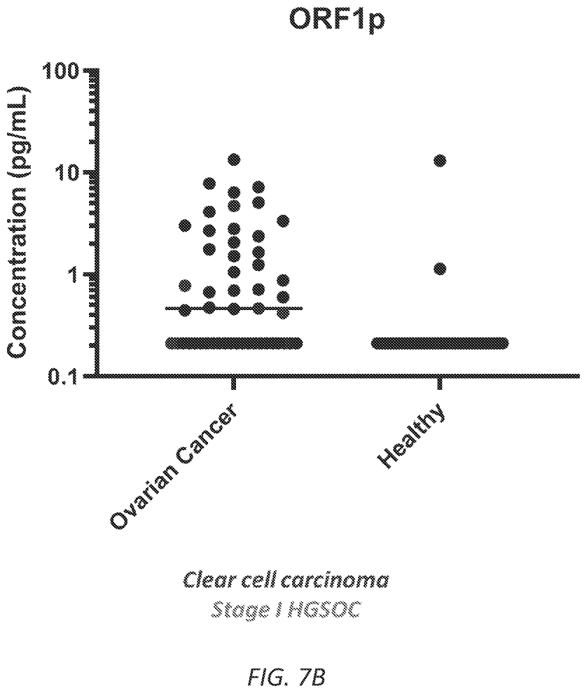

As shown in FIGS. 6 and 7A-B, the results indicated elevated ORF1p levels across multiple cancer types, with detectable levels in about 63% of HGSOC patients (36/57) over two independent pilot cohorts. Importantly, the data show that circulating ORF1p is highly specific: ORF1p levels were undetectable (4×LOD (accounting for the dilution factor) was 0.28 μg/mL) in nearly all of over 400 healthy individuals across independent cohorts (FIG. 7); four were positive; one of those was found to have prostate cancer, and limited information is available about the other patients, giving >99% specificity.

The undetectable (below 0.28 μg/mL (accounting for 4× dilution factor), using 25 uL plasma or serum per replicate, diluted fourfold to a total volume of 100 uL) ORF1p baseline levels among the vast majority of healthy individuals therefore facilitates robust thresholding for distinguishing healthy from cancer patients, in contrast to many existing ovarian cancer protein biomarkers, which can have variable baseline levels among individuals. These results thus established strong support for the feasibility and transformative potential of ORF1p as a blood-based biomarker in a number of types of cancer, including ovarian, pancreatic, liver, colorectal, lung, and head and neck cancer.

### Example 2. Improved Assays—Second Generation

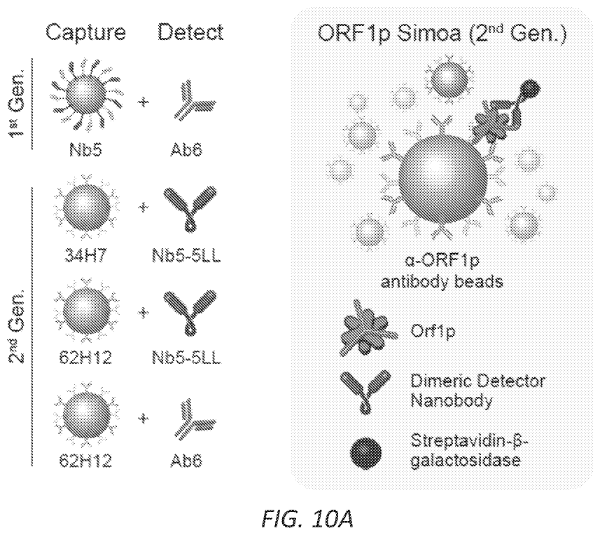

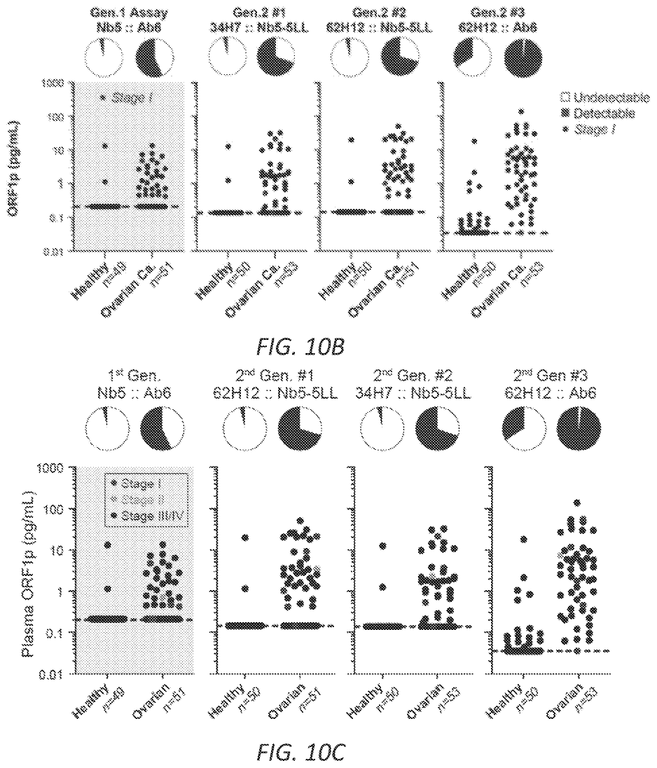

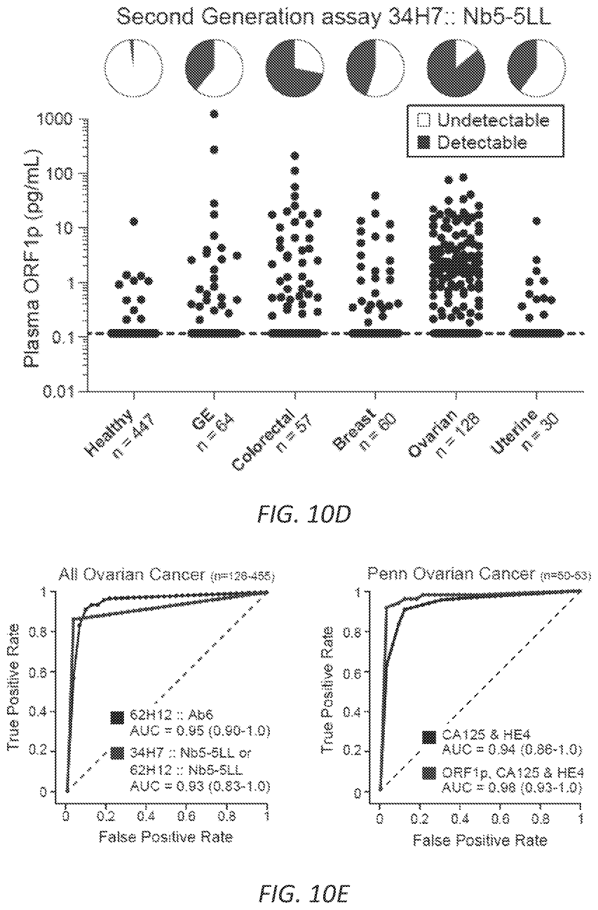

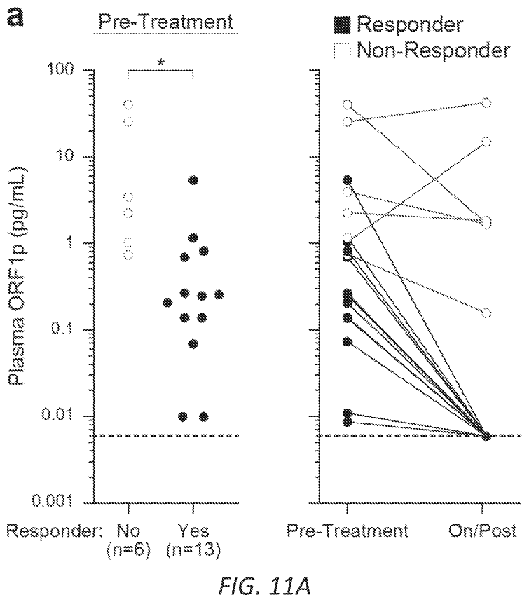

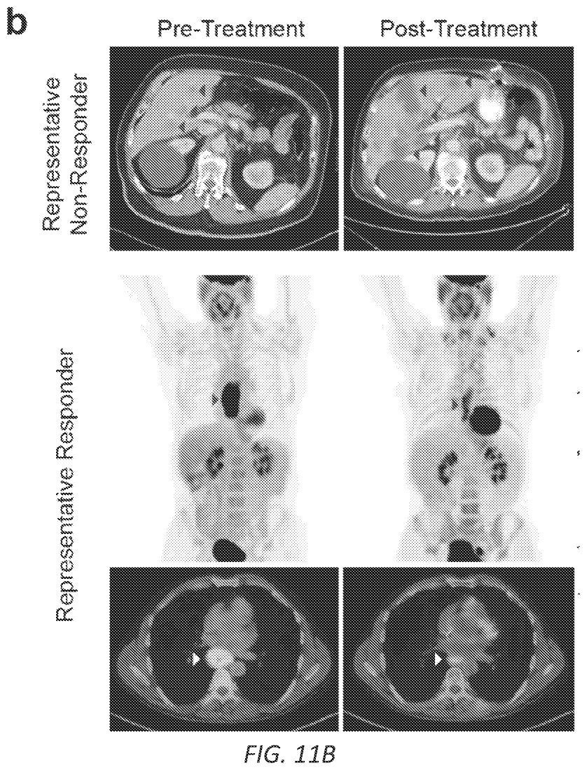

Improved “2nd-generation assays” were developed using custom reagents that improved analytical sensitivity ˜2-5-fold and now detect ORF1p to ˜50 attomolar sensitivity, corresponding to 1000 ORF1 homotrimers out of ˜1019 molecules in 25 μL plasma. These assays employ optimized buffers and rabbit mAbs for capture and either a mAb or an engineered bivalent nanobody “Nb5-5LL” detector, which consists of two linked Nb5 molecules. We applied these assays, along with our “first generation assay” to a pilot cohort of 53 ovarian cancer and 50 healthy patient plasmas. These results (FIG. 10A) demonstrated detection of ˜50% of patients in the first-generation assay, including 3 of 5 assayed stage I patients; the “second generation assays,” which used the three best-performing capture::detection pairs, rabbit monoclonal antibodies 34H7 and 62H12 as capture reagents and either Ab6 or homodimeric form of Nb5 (Nb5-5LL) as detector, improved detection to 70-80% and 4 of 5 stage I patients while retaining high specificity (FIGS. 10A-D). Adding detergent further improved performance, presumably by limiting bead aggregation and improving bead loading into microwells. These second-generation assays achieved detection limits of 0.016-0.029 pg/mL (130-240 aM trimeric ORF1p), and the four different reagents had predominantly non-overlapping epitopes in binning experiments (34H7 and 62H12 partially overlap).

Somewhat unexpectedly, analytical sensitivity of the assay (for detecting recombinant ORF1p in buffer) did not perfectly correspond to clinical sensitivity (for detecting ORF1p in cancer patient plasma). While the second-generation assays demonstrated less than an order-of-magnitude improvement in analytical sensitivity over the first-generation assay, they showed considerable improvement in circulating ORF1p detectability over background in buffer in re-measured samples across a large cohort of healthy and cancer patients. This difference may be due to differing accessibilities of circulating ORF1p epitopes or to different nonspecific binding patterns in plasma.

We next applied these assays to a larger cohort of Stage III and Stage IV MGH ovarian and gastroesophageal cancer patients (FIG. 10B). In ovarian cancer, we saw similar or higher detection rates across a larger cohort with the improved assays, including detection of mucinous ovarian cancer subtypes that tend not to have elevated CA-125. In the esophagus, we demonstrated increased detection rates. Together, these results show early detection of ovarian cancer, including 80% of stage I ovarian patients at clinical diagnosis, and utility for detecting esophageal cancer.

Undetectable or extremely low ORF1p levels in healthy individuals could readily be discriminated from measured ORF1p levels in ovarian cancer patients, resulting in a strong discriminatory ability with single-marker models (area under the receiver operating characteristic curve, AUCs of 0.93 to 0.948, sensitivity of 41% to 81% at 98% specificity, FIG. 10D). This large cohort included pre-treatment plasma samples from a sub-cohort of ovarian cancer patients (mostly high-grade serous ovarian carcinoma, “Penn cohort”) with age-matched controls (n=51-53 women, FIG. 10C); again, second-generation assays showed higher sensitivities while maintaining high specificities, notably achieving detection of five out of six Stage I/II patients at >98% specificity. Furthermore, multivariate models combining ORF1p (34H7::Nb5-5LL assay) with ovarian cancer biomarkers CA125 and HE4 yielded improved diagnostic performance over these existing markers (CA125 and HE4 alone, AUC=0.94, 59% sensitivity at 98% specificity; ORF1p, CA125, and HE4, AUC=0.98, 91% sensitivity at 98% specificity; FIG. 10D). While it is not clear whether the low ORF1p levels detected in several healthy individuals is due to nonspecific binding, true background levels of ORF1p, or an unappreciated pre-malignant state, several positive healthy controls were positive by only one of the three second-generation assays (n=4 positive by only 62H12::Nb5-5LL and n=75 positive by only 62H12:Ab6), suggesting nonspecific binding in at least some of these cases and the potential to improve specificity by combining data from multiple assays. Our results indicate that by developing improved affinity reagents, we achieved improved clinical sensitivity in detecting circulating ORF1p in cancer patients, with 83% sensitivity at >98% specificity towards early detection of ovarian cancer.

Receptor subtypes were available for the breast cancer cohort, which included 30 patients each with metastatic and localized disease. Across all assays, triple negative tended to have higher positivity rates, but the most sensitive 2^{nd} generation assay (62H12::Ab6) detected 96% of triple negative cases and 91% of the remaining cases with 93% sensitivity for both localized and metastatic disease. Overall, metastatic disease was detected more commonly than localized disease (43% vs 6.7% for 1^{st} generation assay, 67-93% vs. 23-93% for 2^{nd} generation assays, depending on the assay), and all three 2^{nd} generation assays had higher sensitivity than the 1^{st} generation assay.

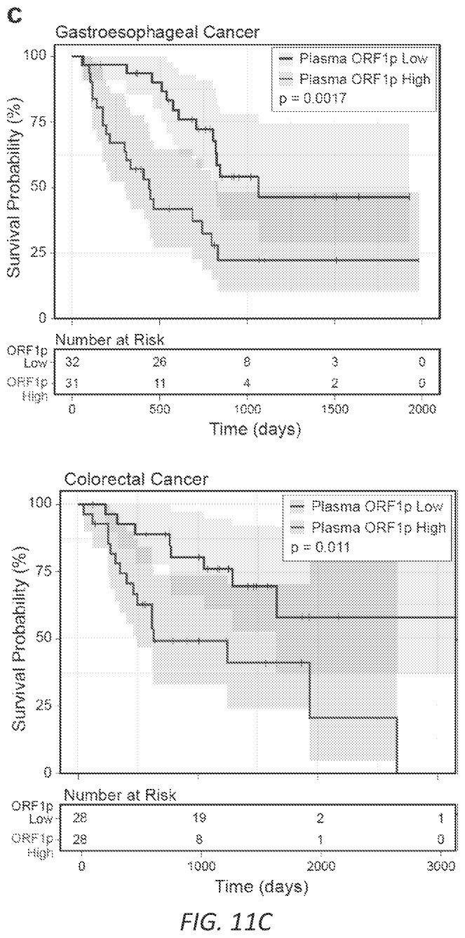

To test whether ORF1p might be useful for monitoring therapeutic response, 19 patients with gastroesophageal cancer were identified who had both detectable plasma ORF1p at diagnosis as well as subsequent samples available collected during or after treatment (average 80 days after initiation of therapy, range 26-179 days). Primary tumors were all adenocarcinoma and located in the esophagus (n=7), gastroesophageal junction (n=7) and stomach (n=5). All patients received systemic therapy, chemotherapy or chemoradiotherapy: CROSS (n=3), FLOT (n=3), FOLFIRINOX (n=2), FOLFOX (n=9), FOLFOX+trastuzumab (n=1), or FOLFOX then CROSS (n=1). A smaller fraction of patients also received radiation and/or surgery. Clinical response (‘Responders’ and ‘Non-Responders’) was determined by review of re-staging CT and PET-CT imaging by clinicians blinded to the assay results. Over an average of 465 days (range 98-1098), 12 patients died, six were alive at last follow-up (all ‘Responders’), and one was lost to follow-up. Non-Responders had higher pre-treatment plasma ORF1p (FIG. 11A, left panel, p=0.02). All 6 patients with detectable ORF1p at follow-up sampling, as defined by positivity over background in two of three assays, were also Non-Responders by imaging (FIG. 11A, right panel, p<0.0001, Fisher's Exact test) and had reduced survival (p=0.001 log-rank test for overall survival). In contrast, in all 13 Responders, circulating ORF1p dropped to undetectable levels at follow-up sampling. Plasma ORF1p in four Responders and two Non-Responders was measured at an early timepoint of 26-33 days. The timing of sampling was not different between groups (average 93 days for Non-Responders, 74 for Responders, p=0.5). Pre-therapy blood was drawn on an average of 20 days after diagnosis (range −8-48, average 22 for Non-Responders and 19 for Responders, p=0.6). Representative PET and PET-CT images are shown (FIG. 11B), both images are taken approximately two months after initiation of therapy, a month after the plasma ORF1p result. Thus, reduction in circulating ORF1p paralleled treatment response and survival, while persistent circulating ORF1p corresponded to patients with refractory disease, indicating the predictive potential of this marker.

Because these results indicated that pre-treatment plasma ORF1p levels might be prognostic, we evaluated the prognostic value of 2^{nd} generation ORF1p Simoa assays in our cohorts of GE, CRC, and ovarian cancer patients. We stratified the patients based on either the median ORF1p value or ORF1p detectability (methods) and found that higher pre-therapy plasma ORF1p was significantly associated with poor survival in GE and CRC (FIG. 11C, p=0.0017 and 0.011, log rank test, respectively) but not in ovarian cancer. ORF1p remained significantly prognostic in multivariate analysis in GE and CRC.

### Example 3. Increasing Analytical Sensitivity

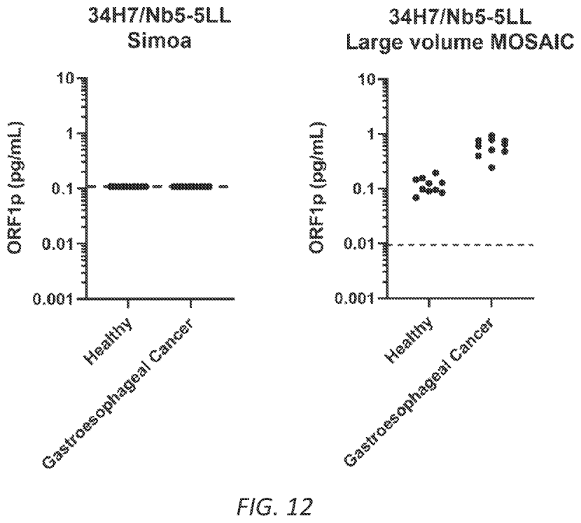

It was hypothesized that the assay described in Example 1 was limited by the number of ORF1p molecules in a 25 μL sample; using larger volumes of patient plasma could increase the number of target molecules present. For example, a teaspoon (5 ml) is 200-fold more than used in a 25 μL assay sample. Additionally, a recently developed flow-based detection platform Molecular On-bead Signal Amplification for Individual Counting (MOSAIC) may improve sensitivity.³⁰ A cohort using 20-fold more volume was evaluated. 0.5 ml of plasma (500 μl) was diluted 1:4 in a Diluent binding buffer containing additionally 1% Triton X-100 detergent and bound to 34H7 conjugated beads for 90 minutes, washed, detected by binding biotinylated Nb5-5LL (also known as MT997) assayed using MOSAIC for on-bead signal development with read-out on a flow cytometer. As see in FIG. 12, 9 of 10 previously undetectable patients showed signal above healthy controls.

We collect larger volumes (3-10 ml) from 20 HGSOC patients, 20 controls with benign conditions, 20 EAC, and 20 Barrett's Esophagus patients with matched tissue to provide “ground truth” on ORF1p expression, as well as large volume healthy plasma and large volume gastroesophageal plasmas. MOSAIC assays are performed using reagents described herein, variables including selection of reagents, buffer additives such as bead number and loading, salt and detergent, binding times, and wash conditions are varied, optimizing sensitivity, specificity, and reproducibility.

### Example 3. Development of Nanobodies

Using methods as described in Fridy et al., 2014⁴⁹, nanobodies that bind to ORF1p were generated. Briefly, highly purified ORF1p was generated from E. coli as in Carter et al., 2020³⁵, a llama was immunized, bone marrow extracted and candidate DNA regions amplified and sequenced to generate a library, serum was fractionated and then bound to immobilized purified ORF1p, bound heavy-chain antibodies were eluted and VHH fragments extracted and sequenced by mass spectrometry using a library derived from the aforementioned bone marrow library. Candidate sequences were then assembled, synthesized, cloned, and screened for the ability to bind ORF1p. Identified sequences are shown in Table B.

TABLE B
Nanobodies (CDRs in bold font)

| Description | Sequence | CDR1 | CDR2 | CDR3 |
|---|---|---|---|---|
| anti-ORF1 | DVQLVESGGGLVQAGESLRLSCAASGRTLSSL | GRTLSSLN | ISRSGST | AADDNY |
| Nanobody 1 | NMAWFRQVPGKERDFVSLISRSGSTDYVHSV | SEQ ID | (SEQ ID | NGRYPFR |
| (Nb1) | RGRFTISRDNAKNTVYLEMNSLKPEDTAVYYC | NO: 22) | NO: 23) | DEYEY |
| | AADDNYNGRYPFRDEYEYWGQGTQVTVS | | | (SEQ ID |
| | (SEQ ID NO:21) | | | NO:24) |
| anti-ORF1 | DVQLVESGGGLVQAGGSLRLSCAASGRTDDI | GRTDDI | ISWSITR | AAKTGYS |
| Nanobody 2 | YTMGWFRQGPGKEREFVAVISWSITRRSSIT | YT (SEQ | RSSIT | GSMSSYQ |
| (Nb2) | HYADSVKGRFTISGENAENTVYLQMNTLKPED | ID | (SEQ ID | DYDS |
| | TAVYYCAAKTGYSGSMSSYQDYDSWGQGT | NO:26) | NO:27) | (SEQ ID |
| | QVTVS (SEQ ID NO:25) | | | NO:28) |
| anti-ORF1 | QVQLVESGGDLVQAGGSLRLSCAVSGGTSSN | GGTSSN | ISWSGSR | TAVREYR |
| Nanobody 5 | YGMGWFRQAPGKEREFVSSISWSGSRTLYSD | YG (SEQ | T (SEQ ID | DYPQRDN |
| (Nb5) | SVKGRFTISRDNAKNTVDLQMNSLKPEDTAVY | ID | NO:3) | FDY (SEQ |
| | YCTAVREYRDYPQRDNFDYWGQGTQVTVS | NO:2) | | ID NO:4) |
| | (SEQ ID NO:1) | | | |
| anti-ORF1 | QVHLVESGGKLVQAGESLKLSCEVSGRTFSISG | GRTFSIS | ITWTGGY | ATRRAGD |
| Nanobody 9 | AGWFRQAPGKEREFVAAITWTGGYPYYDAS | G (SEQ | P (SEQ ID | LDFSYAQ |
| (Nb9) | VKGRFTISREDPDNTVYLSMNSLKPEDTAVYY | ID | NO:31) | QGRYDN |
| | CATRRAGDLDFSYAQQGRYDNWGQGTQVT | NO:30) | | (SEQ ID |
| | VS (SEQ ID NO:29) | | | NO:32) |
| anti-ORF1 | QVQLVESGGGLVQAGESLRLSCQVSGRTFSIS | GRTFSIS | ITWTGGY | ATRRAGS |
| Nanobody 10 | GAGWFRQAPGKEREFVAAITWTGGYPYYATS | G (SEQ | P (SEQ | LDFDYAQ |
| (Nb10) | VKGRFTIARDDPENRVYLSMNSLKPEDTAVYY | ID | ID | QTRYDN |
| | CATRRAGSLDFDYAQQTRYDNWGQGTQVT | NO:34) | NO:35) | (SEQ ID |
| | VS (SEQ ID NO:33) | | | NO:36) |
| anti-ORF1 | QVQLAESGGGLVQAGGSLRLSCAASGRTSTIY | GRTSTIY | ITYTGGY | AARSRGG |
| Nanobody 21 | IMGWFRQAPGKDREFVAGITYTGGYPYYADS | I (SEQ | P (SEQ | ITTHERDF |
| (Nb21) | VQGRFTLSNDNADHTAYLQMNNLKPEDSAV | ID | ID | GS (SEQ |
| | YYCAARSRGGITTHERDFGSWGQGTQVTVS | NO:38) | NO:39) | ID |
| | (SEQ ID NO:37) | | | NO:40) |

NB5 (also referred to herein as Clone 5) was selected for further development.

An exemplary nucleic acid encoding Nb5 is as follows:

(SEQ ID NO: 41)
CAGGTACAGCTTGTGGAATCAGGGGGTGACCTTGTGCAGGCAGGAGGGTCACTGCGCTTATCTTGTGCGGT
CAGTGGGGGCACGTCATCAAACTACGGGATGGGTTGGTTTCGTCAAGCCCCTGGAAAGGAGCGCGAGTTTG
TCTCATCGATCTCCTGGTCAGGCAGTCGTACTTTATATAGCGACTCAGTGAAAGGCCGCTTCACGATTAGT
CGTGATAATGCGAAAAACACCGTTGACTTGCAGATGAACTCTTTGAAGCCTGAAGACACGGCAGTCTATTA
TTGCACCGCAGTACGCGAGTATCGCGACTACCCGCAGCGCGATAACTTTGACTATTGGGGACAAGGGACGC
AGGTTACGGTAAGT

Because ORF1p is a homotrimer, many possible high-affinity combinations of binding reagents are possible. Further screening resulted in 21 high affinity clones after two rounds of affinity mass spectrometry and cloning,⁴⁹ the best of which had mid-picomolar affinities (Table 1), and clones with non-overlapping epitopes were identified. As seen in Table 1, the engineered nanobody reagents had low-picomolar affinities, similar to or surpassing existing antibody reagents (Ab6, 4H1, and Ab54. A first round of engineering dimeric and trimeric reagents with linker length optimization (a “long-linker” of 56 residues containing both flexible (GGGGS) (SEQ ID NO:6) and rigid (EAAAK) (SEQ ID NO:11) segments was most advantageous, a linker of 20 flexible (GGGGS) (SEQ ID NO:6) residues was also advantageous) resulted in improved affinity, with two reagents outperforming the prior best mAb, Ab6. Table C provides sequences of some of the concatemer constructs developed.

TABLE C
Exemplary Concatemer Constructs

| Description | Sequence |
|---|---|
| anti-ORF1 Nb | DVQLVESGGGLVQAGESLRLSCAASGRTLSSLNMAWFRQVPGK |
| dimer 1 Nb1- | ERDFVSLISRSGSTDYVHSVRGRFTISRDNAKNTVYLEMNSLKPED |
| Nb1 (MT991) | TAVYYCAADDNYNGRYPFRDEYEYWGQGTQVTVSGGGGSGGG |
| | GSGGGGSGGGGSMADVQLVESGGGLVQAGESLRLSCAASGRTL |
| | SSLNMAWFRQVPGKERDFVSLISRSGSTDYVHSVRGRFTISRDNA |
| | KNTVYLEMNSLKPEDTAVYYCAADDNYNGRYPFRDEYEYWGQG |
| | TQVTV (SEQ ID NO:42) |
| anti-ORF1 Nb | DVQLVESGGGLVQAGGSLRLSCAASGRTDDIYTMGWFRQGPGK |
| dimer 2 Nb2- | EREFVAVISWSITRRSSITHYADSVKGRFTISGENAENTVYLQMNT |
| Nb2 (MT992) | LKPEDTAVYYCAAKTGYSGSMSSYQDYDSWGQGTQVTVSGGG |
| | GSGGGGSGGGGSGGGGSMADVQLVESGGGLVQAGGSLRLSCA |
| | ASGRTDDIYTMGWFRQGPGKEREFVAVISWSITRRSSITHYADSV |
| | KGRFTISGENAENTVYLQMNTLKPEDTAVYYCAAKTGYSGSMSS |
| | YQDYDSWGQGTQVTVS* (SEQ ID NO:43) |
| anti-ORF1 Nb | QVQLVESGGDLVQAGGSLRLSCAVSGGTSSNYGMGWFRQAPG |
| dimer 3 Nb5- | KEREFVSSISWSGSRTLYSDSVKGRFTISRDNAKNTVDLQMNSLK |
| Nb5 (MT993) | PEDTAVYYCTAVREYRDYPQRDNFDYWGQGTQVTVSGGGGSG |
| | GGGSGGGGSGGGGSMAQVQLVESGGDLVQAGGSLRLSCAVSG |
| | GTSSNYGMGWFRQAPGKEREFVSSISWSGSRTLYSDSVKGRFTIS |
| | RDNAKNTVDLQMNSLKPEDTAVYYCTAVREYRDYPQRDNFDY |
| | WGQGTQVTVS (SEQ ID NO:44) |
| anti-ORF1 Nb | QVQLVESGGDLVQAGGSLRLSCAVSGGTSSNYGMGWFRQAPG |
| dimer 4 Nb5- | KEREFVSSISWSGSRTLYSDSVKGRFTISRDNAKNTVDLQMNSLK |
| Nb1 (MT994) | PEDTAVYYCTAVREYRDYPQRDNFDYWGQGTQVTVSGGGGSG |
| | GGGSGGGGSGGGGSMADVQLVESGGGLVQAGESLRLSCAASG |
| | RTLSSLNMAWFRQVPGKERDFVSLISRSGSTDYVHSVRGRFTISR |
| | DNAKNTVYLEMNSLKPEDTAVYYCAADDNYNGRYPFRDEYEYW |
| | GQGTQVTVS (SEQ ID NO:45) |
| anti-ORF1 Nb | QVQLVESGGDLVQAGGSLRLSCAVSGGTSSNYGMGWFRQAPG |
| dimer 5 Nb5- | KEREFVSSISWSGSRTLYSDSVKGRFTISRDNAKNTVDLQMNSLK |
| Nb2 (MT995) | PEDTAVYYCTAVREYRDYPQRDNFDYWGQGTQVTVSGGGGSG |
| | GGGSGGGGSGGGGSMADVQLVESGGGLVQAGGSLRLSCAASG |
| | RTDDIYTMGWFRQGPGKEREFVAVISWSITRRSSITHYADSVKGR |
| | FTISGENAENTVYLQMNTLKPEDTAVYYCAAKTGYSGSMSSYQD |
| | YDSWGQGTQVTVS (SEQ ID NO:46) |
| anti-ORF1 Nb | QVQLVESGGDLVQAGGSLRLSCAVSGGTSSNYGMGWFRQAPG |
| trimer Nb5- | KEREFVSSISWSGSRTLYSDSVKGRFTISRDNAKNTVDLQMNSLK |
| Nb5-Nb5 | PEDTAVYYCTAVREYRDYPQRDNFDYWGQGTQVTVSGGGGSG |
| (MT996) | GGGSGGGGSGGGGSMAQVQLVESGGDLVQAGGSLRLSCAVSG |
| | GTSSNYGMGWFRQAPGKEREFVSSISWSGSRTLYSDSVKGRFTIS |
| | RDNAKNTVDLQMNSLKPEDTAVYYCTAVREYRDYPQRDNFDY |
| | WGQGTQVTVSGGGGSGGGGSGGGGSGGGGSMAQVQLVESG |
| | GDLVQAGGSLRLSCAVSGGTSSNYGMGWFRQAPGKEREFVSSIS |
| | WSGSRTLYSDSVKGRFTISRDNAKNTVDLQMNSLKPEDTAVYYC |
| | TAVREYRDYPQRDNFDYWGQGTQVTVS (SEQ ID NO:47) |
| anti-ORF1 Nb | QVQLVESGGDLVQAGGSLRLSCAVSGGTSSNYGMGWFRQAPG |
| dimer 6 Nb5- | KEREFVSSISWSGSRTLYSDSVKGRFTISRDNAKNTVDLQMNSLK |
| Nb5 long | PEDTAVYYCTAVREYRDYPQRDNFDYWGQGTQVTVSGGGGSG |
| linker (MT997) | GGGSGGGGSGGGGSEAAAKEAAAKEAAAKSGGGGSGGGGSG |
| | GGGSGGGGSMAQVQLVESGGDLVQAGGSLRLSCAVSGGTSSN |
| | YGMGWFRQAPGKEREFVSSISWSGSRTLYSDSVKGRFTISRDNA |
| | KNTVDLQMNSLKPEDTAVYYCTAVREYRDYPQRDNFDYWGQG |
| | TQVTVS (SEQ ID NO:48) |
| Nb5-Nb5-7Cys | QVQLVESGGDLVQAGGSLRLSCAVSGGTSSNYGMGWFRQAPG |
| anti-ORF1 Nb | KEREFVSSISWSGSRTLYSDSVKGRFTISRDNAKNTVDLQMNSLK |
| dimer 7 (short | PEDTAVYYCTAVREYRDYPQRDNFDYWGQGTQVTVSGGGGSG |
| linker) | GGGSGGGGSGGGGSMAQVQLVESGGDLVQAGGSLRLSCAVSG |
| (MT1032) | GTSSNYGMGWFRQAPGKEREFVSSISWSGSRTLYSDSVKGRFTIS |
| | RDNAKNTVDLQMNSLKPEDTAVYYCTAVREYRDYPQRDNFDY |
| | WGQGTQVTVSCGSGRCGSGRCGSGRCGSGRC (SEQ ID |
| | NO:49) |
| Nb5-Nb5-7Cys | QVQLVESGGDLVQAGGSLRLSCAVSGGTSSNYGMGWFRQAPG |
| anti-ORF1 Nb | KEREFVSSISWSGSRTLYSDSVKGRFTISRDNAKNTVDLQMNSLK |
| dimer 8 (long | PEDTAVYYCTAVREYRDYPQRDNFDYWGQGTQVTVSGGGGSG |
| linker v1) | GGGSGGGGSGGGGSEAAAKEAAAKEAAAKSGGGGSGGGGSG |
| (MT1033) | GGGSGGGGSMAQVQLVESGGDLVQAGGSLRLSCAVSGGTSSN |
| | YGMGWFRQAPGKEREFVSSISWSGSRTLYSDSVKGRFTISRDNA |
| | KNTVDLQMNSLKPEDTAVYYCTAVREYRDYPQRDNFDYWGQG |
| | TQVTVSCGSGRCGSGRCGSGRCGSGRC (SEQ ID NO:50) |
| Nb2-Nb2-7Cys | DVQLVESGGGLVQAGGSLRLSCAASGRTDDIYTMGWFRQGPGK |
| anti-ORF1 Nb | EREFVAVISWSITRRSSITHYADSVKGRFTISGENAENTVYLQMNT |
| dimer 9 (long | LKPEDTAVYYCAAKTGYSGSMSSYQDYDSWGQGTQVTVSGGG |
| linker v2) | GSGGGGSGGGGSGGGGSDAAARDAAARDAAARGGGGSGGG |
| (MT1034) | GSGGGGSGGGGSADVQLVESGGGLVQAGGSLRLSCAASGRTDD |
| | IYTMGWFRQGPGKEREFVAVISWSITRRSSITHYADSVKGRFTISG |
| | ENAENTVYLQMNTLKPEDTAVYYCAAKTGYSGSMSSYQDYDSW |
| | GQGTQVTVSCGSGRCGSGRCGSGRCGSGRC (SEQ ID NO:51) |
| Nb5-Nb5-7Cys | QVQLVESGGDLVQAGGSLRLSCAVSGGTSSNYGMGWFRQAPG |
| anti-ORF1 Nb | KEREFVSSISWSGSRTLYSDSVKGRFTISRDNAKNTVDLQMNSLK |
| dimer 10 (long | PEDTAVYYCTAVREYRDYPQRDNFDYWGQGTQVTVSGGGGSG |
| linker v2) | GGGSGGGGSGGGGSDAAARDAAARDAAARGGGGSGGGGSG |
| (MT1035) | GGGSGGGGSMAQVQLVESGGDLVQAGGSLRLSCAVSGGTSSN |
| | YGMGWFRQAPGKEREFVSSISWSGSRTLYSDSVKGRFTISRDNA |
| | KNTVDLQMNSLKPEDTAVYYCTAVREYRDYPQRDNFDYWGQG |
| | TQVTVSCGSGRCGSGRCGSGRCGSGRC (SEQ ID NO:52) |
| Nb2-Nb9-7Cys | DVQLVESGGGLVQAGGSLRLSCAASGRTDDIYTMGWFRQGPGK |
| anti-ORF1 Nb | EREFVAVISWSITRRSSITHYADSVKGRFTISGENAENTVYLQMNT |
| dimer 11 (long | LKPEDTAVYYCAAKTGYSGSMSSYQDYDSWGQGTQVTVSGGG |
| linker v2) | GSGGGGSGGGGSGGGGSDAAARDAAARDAAARGGGGSGGG |
| (MT1036) | GSGGGGGGGGSGSMAQVHLVESGGKLVQAGESLKLSCEVSGR |
| | TFSISGAGWFRQAPGKEREFVAAITWTGGYPYYDASVKGRFTISR |
| | EDPDNTVYLSMNSLKPEDTAVYYCATRRAGDLDFSYAQQGRYD |
| | NWGQGTQVTVSCGSGRCGSGRCGSGRCGSGRC (SEQ ID |
| | NO:53) |
| Nb5-Nb9-7Cys | QVQLVESGGDLVQAGGSLRLSCAVSGGTSSNYGMGWFRQAPG |
| anti-ORF1 Nb | KEREFVSSISWSGSRTLYSDSVKGRFTISRDNAKNTVDLQMNSLK |
| dimer 12 (long | PEDTAVYYCTAVREYRDYPQRDNFDYWGQGTQVTVSGGGGSG |
| linker v2) | GGGSGGGGSGGGGSDAAARDAAARDAAARGGGGSGGGGSG |
| (MT1037) | GGGSGGGGSGSMAQVHLVESGGKLVQAGESLKLSCEVSGRTFSI |
| | SGAGWFRQAPGKEREFVAAITWTGGYPYYDASVKGRFTISREDP |
| | DNTVYLSMNSLKPEDTAVYYCATRRAGDLDFSYAQQGRYDNWG |
| | QGTQVTVSCGSGRCGSGRCGSGRCGSGRC (SEQ ID NO:54) |
| Nb9-Nb9-7Cys | QVHLVESGGKLVQAGESLKLSCEVSGRTFSISGAGWFRQAPGKE |
| anti-ORF1 Nb | REFVAAITWTGGYPYYDASVKGRFTISREDPDNTVYLSMNSLKPE |
| dimer 13 (long | DTAVYYCATRRAGDLDFSYAQQGRYDNWGQGTQVTVSGGGGS |
| linker v2) | GGGGSGGGGSGGGGSDAAARDAAARDAAARGGGGSGGGGS |
| (MT1038) | GGGGSGGGGSGSMAQVHLVESGGKLVQAGESLKLSCEVSGRTF |
| | SISGAGWFRQAPGKEREFVAAITWTGGYPYYDASVKGRFTISRED |
| | PDNTVYLSMNSLKPEDTAVYYCATRRAGDLDFSYAQQGRYDNW |
| | GQGTQVTVSCGSGRCGSGRCGSGRCGSGRC (SEQ ID NO:55) |
| Nb5-Nb2-Nb9- | QVQLVESGGDLVQAGGSLRLSCAVSGGTSSNYGMGWFRQAPG |
| 7Cys anti- | KEREFVSSISWSGSRTLYSDSVKGRFTISRDNAKNTVDLQMNSLK |
| ORF1 Nb | PEDTAVYYCTAVREYRDYPQRDNFDYWGQGTQVTVSGGGGSG |
| trimer 2 | GGGSGGGGSGGGGSDAAARDAAARDAAARGGGGSGGGGSG |
| (MT1039) | GGGSGGGGSADVQLVESGGGLVQAGGSLRLSCAASGRTDDIYT |
| | MGWFRQGPGKEREFVAVISWSITRRSSITHYADSVKGRFTISGEN |
| | AENTVYLQMNTLKPEDTAVYYCAAKTGYSGSMSSYQDYDSWGQ |
| | GTQVTVSGGGGSGGGGSGGGGSGGGGSDAAARDAAARDAAA |
| | RGGGGSGGGGSGGGGSGGGGSGSMAQVHLVESGGKLVQAGE |
| | SLKLSCEVSGRTFSISGAGWFRQAPGKEREFVAAITWTGGYPYYD |
| | ASVKGRFTISREDPDNTVYLSMNSLKPEDTAVYYCATRRAGDLDF |
| | SYAQQGRYDNWGQGTQVTVSCGSGRCGSGRCGSGRCGSGRC |
| | (SEQ ID NO:56) |
| Nb5-Nb9-Nb2- | QVQLVESGGDLVQAGGSLRLSCAVSGGTSSNYGMGWFRQAPG |
| 7Cys anti- | KEREFVSSISWSGSRTLYSDSVKGRFTISRDNAKNTVDLQMNSLK |
| ORF1 Nb | PEDTAVYYCTAVREYRDYPQRDNFDYWGQGTQVTVSGGGGSG |
| trimer 3 | GGGSGGGGSGGGGSDAAARDAAARDAAARGGGGSGGGGSG |
| (MT1040) | GGGSGGGGSGSMAQVHLVESGGKLVQAGESLKLSCEVSGRTFSI |
| | SGAGWFRQAPGKEREFVAAITWTGGYPYYDASVKGRFTISREDP |
| | DNTVYLSMNSLKPEDTAVYYCATRRAGDLDFSYAQQGRYDNWG |
| | QGTQVTVSGGGGSGGGGSGGGGSGGGGSDAAARDAAARDAA |
| | ARGGGGSGGGGSGGGGSGGGGSADVQLVESGGGLVQAGGSL |
| | RLSCAASGRTDDIYTMGWFRQGPGKEREFVAVISWSITRRSSITH |
| | YADSVKGRFTISGENAENTVYLQMNTLKPEDTAVYYCAAKTGYS |
| | GSMSSYQDYDSWGQGTQVTVSCGSGRCGSGRCGSGRCGSGRC |
| | (SEQ ID NO:57) |

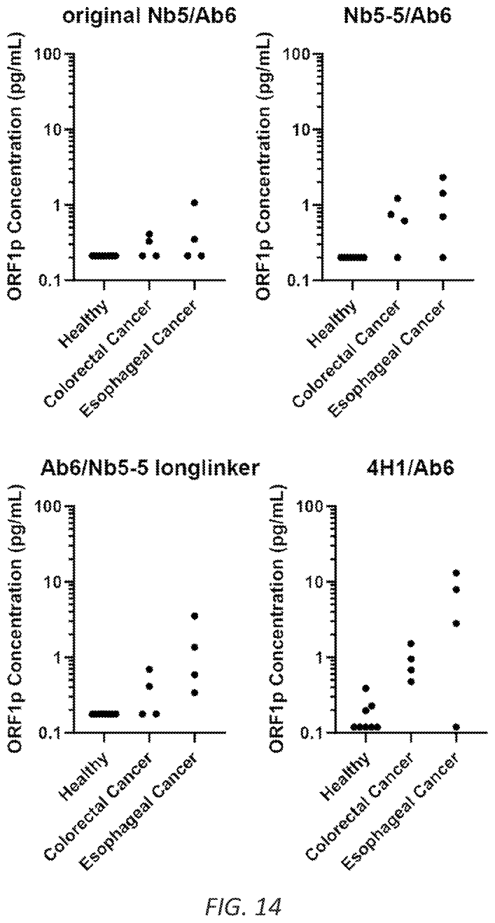

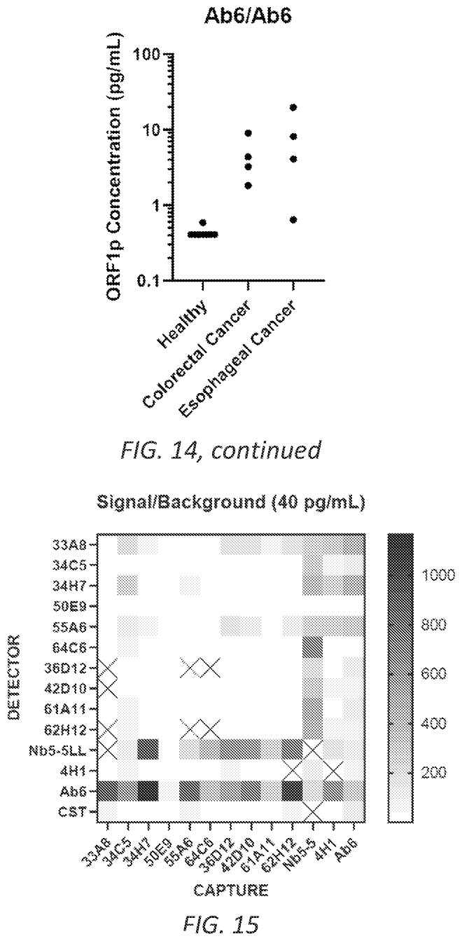

Reagents with non-overlapping epitopes were identified through SPR binning experiments⁴⁹, and combinations were tested empirically through calibration curves with recombinant ORF1p, and best performers were then tested against pilot cohorts of cancer and healthy patient plasma. Combinations of these affinity reagents were tested using Simoa on spiked healthy plasma and showed improvement in limit of detection of up to 7-fold (Table 2). Interestingly, as shown in FIG. 14, which depicts ORF1p measurements of select reagent pairs in a small set of healthy and cancer patient plasma, the highest-affinity reagents were not always the best performers in plasma, so a combination of rational and empiric screening was required.

TABLE 1
Affinity measurements by Surface Plasmon Resonance
(SPR) of anti-ORF1 affinity reagents

| Clone | ID | ka | kd | KD |
|---|---|---|---|---|
| Ab54 | Rabbit Mab | 4.3E+05 | 2.9E−06 | 6.9E−12 |
| Nb 2-2 | Dimeric Nb | 6.3E+05 | 5.8E−06 | 9.2E−12 |
| Nb 5-5 long | Dimeric Nb | 7.3E+05 | 7.2E−06 | 9.8E−12 |
| Ab6 | Rabbit Mab | 2.8E+05 | 4.2E−06 | 1.5E−11 |
| 4H1 | Mouse Mab | 4.0E+05 | 6.5E−06 | 1.6E−11 |
| Nb 5-5-5 | Trimeric Nb | 7.7E+05 | 2.1E−05 | 2.7E−11 |
| Nb 5-2 | Dimeric Nb | 3.6E+05 | 1.3E−05 | 3.6E−11 |
| Nb 5-5 | Dimeric Nb | 8.5E+05 | 6.0E−05 | 7.0E−11 |
| Nb 5-1 | Dimeric Nb | 6.0E+05 | 5.0E−05 | 8.3E−11 |
| Nb-5 | Nanobody | 3.0E+06 | 3.2E−04 | 1.1E−10 |
| Nb-9 | Nanobody | 9.9E+05 | 1.9E−04 | 1.9E−10 |
| Nb-10 | Nanobody | 1.6E+06 | 3.0E−04 | 1.9E−10 |
| Nb-21 | Nanobody | 1.2E+06 | 3.0E−04 | 2.4E−10 |
| Nb-2 | Nanobody | 4.0E+05 | 1.9E−04 | 4.8E−10 |

TABLE 2
Combinations measured by SIMOA

| Capture | Detector | ug/mL detector | LOD (fg/mL) |
|---|---|---|---|
| 4H1 | Nb 5-5 | 0.3 | 7 |
| Ab6 | Ab6 | 0.6 | 19 |
| 4H1 | Ab6 | 0.3 | 30 |
| Ab6 | Nb 5-5 long | 0.3 | 44 |
| Nb 5-5 | Ab6 | 0.3 | 50 |
| Nb5 | Ab6 | 0.3 | 53 |
| 4H1 | Nb 5-5 long | 0.3 | 56 |
| Ab6 | Nb 5-5 | 0.15 | 59 |
| Nb 5-5 | 4H1 | 0.3 | 72 |
| Nb 5-5 long | Ab6 | 0.3 | 75 |
| 4H1 | Nb 5-5 | 0.15 | 91 |
| Ab6 | Nb 5-1 | 0.3 | 101 |
| Ab6 | Ab6 | 0.3 | 102 |
| Ab6 | Nb 5-5 long | 0.15 | 111 |

Simoa optimization using novel and existing reagents yielded candidate assays with up to 7-fold lower limit of detection (LOD) for ORF1p of 7 fg/ml (˜50 aM). Because the target is trimeric, some combinations may yield very high affinity.

### Example 4. Development of Monoclonal Antibodies

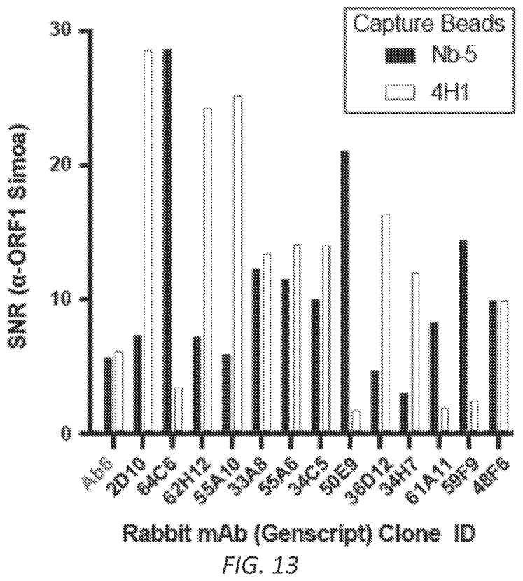

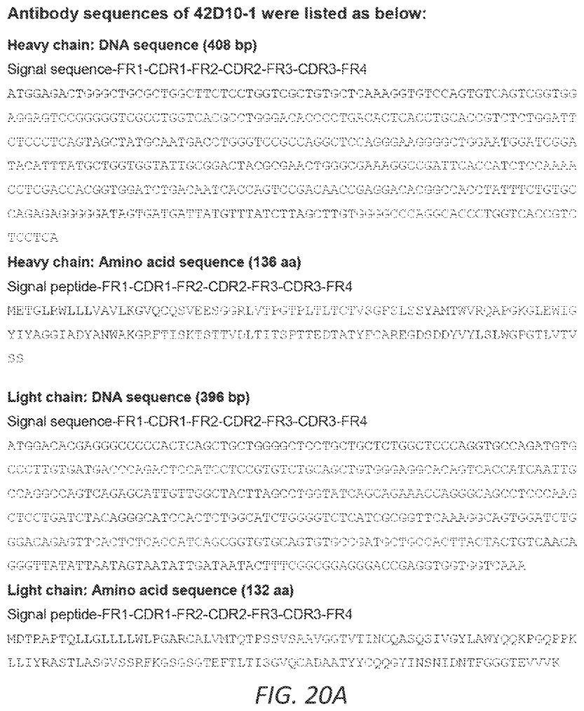

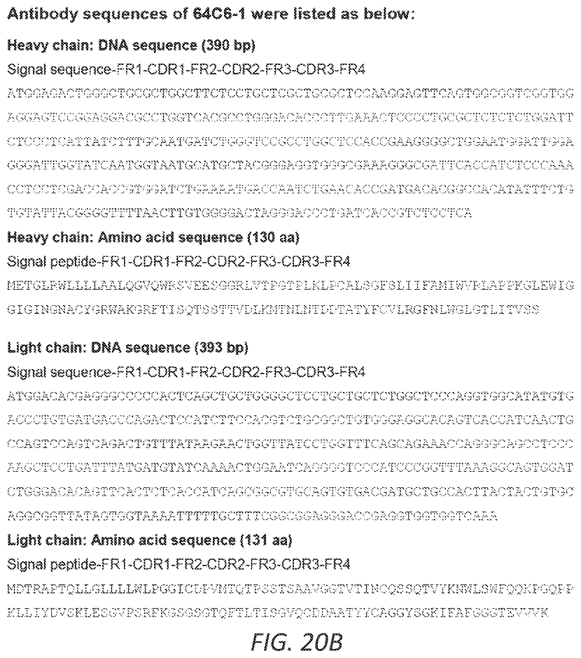

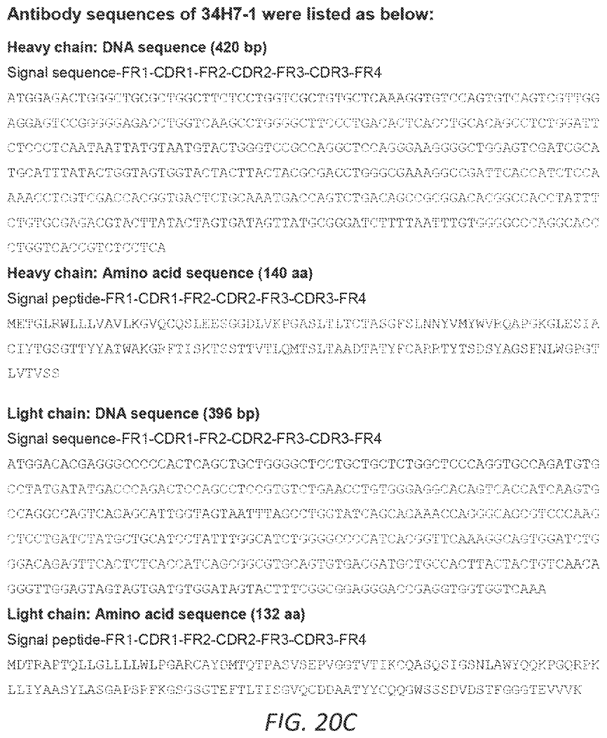

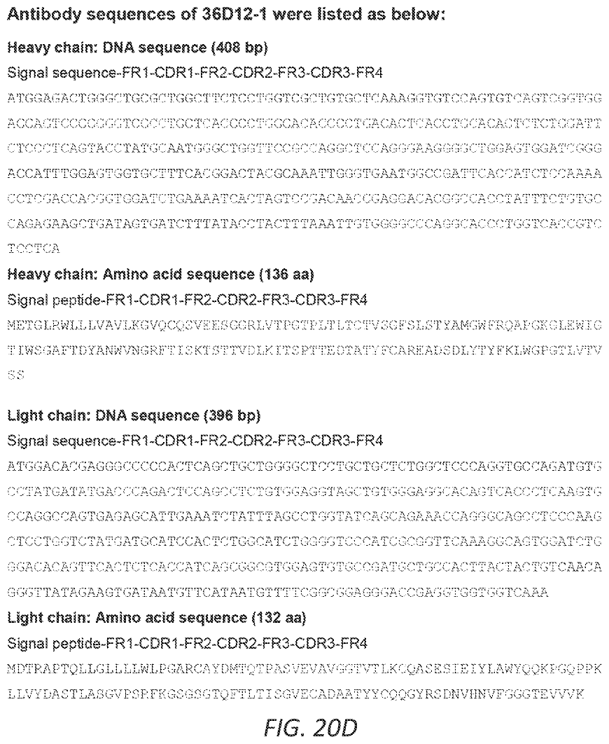

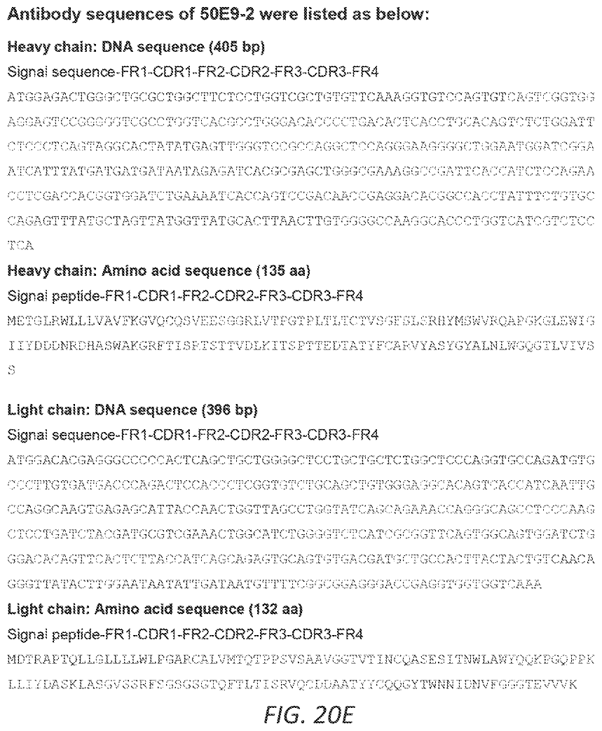

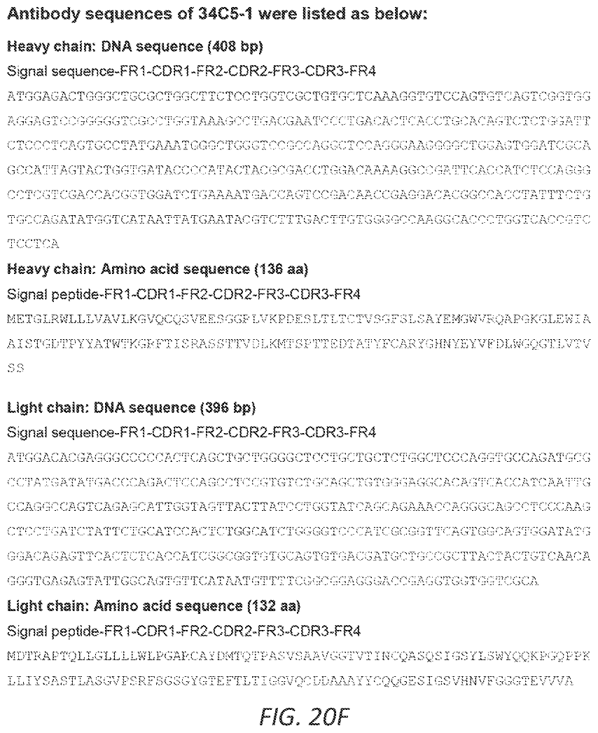

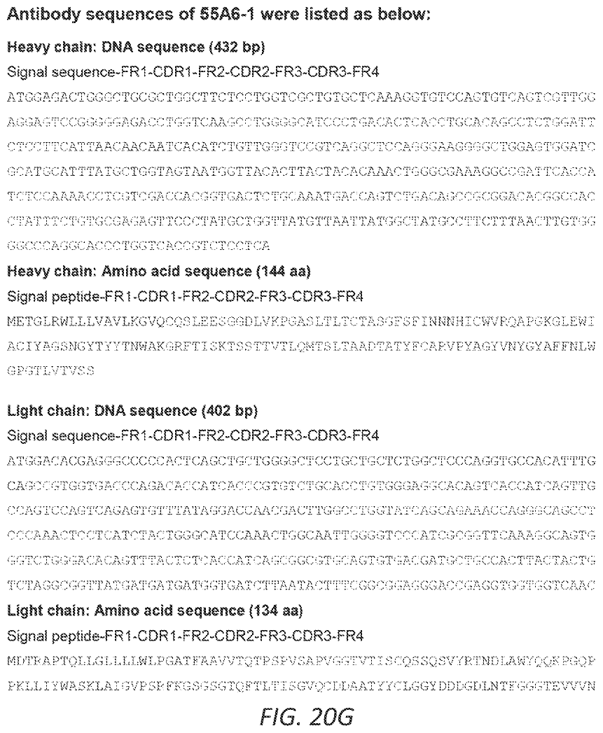

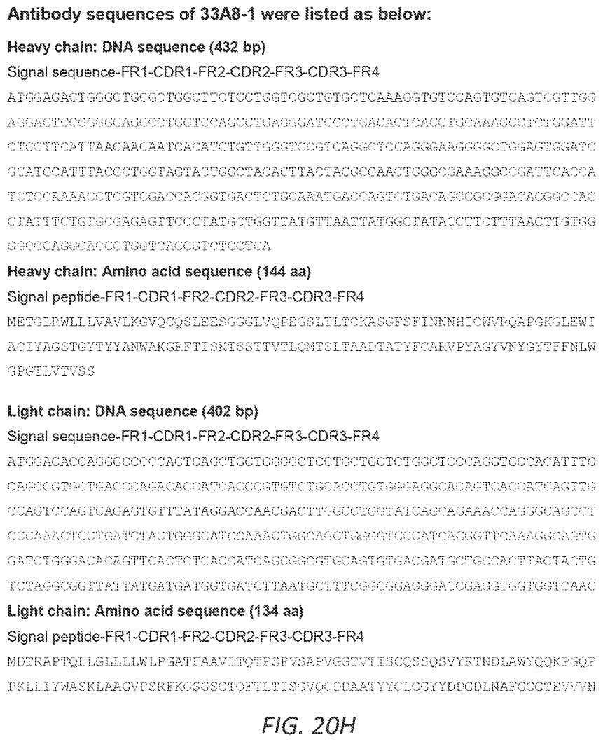

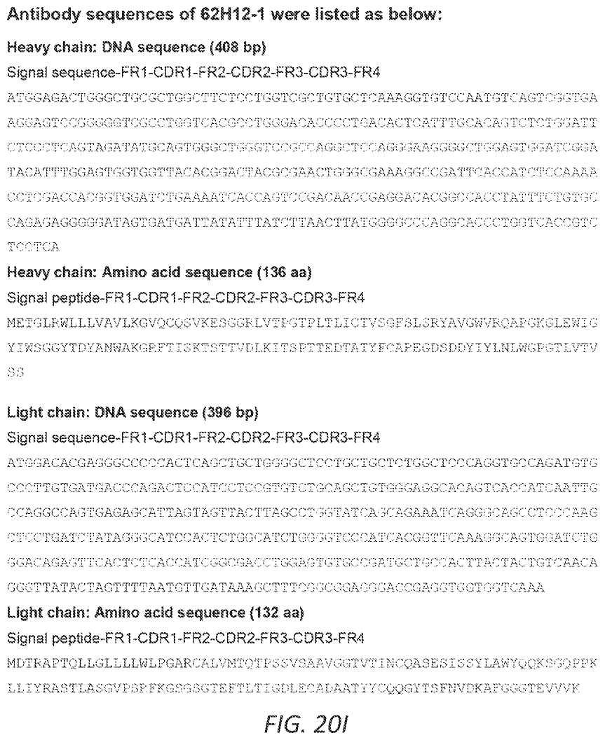

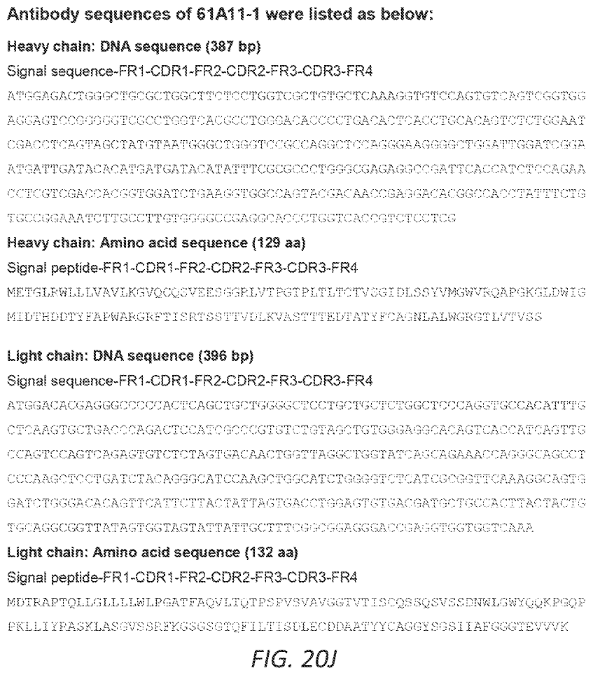

GenScript's MonoRab™ immunization and B cell cloning platform was used with purified, recombinant human ORF1p trimers. The 50 best-performing clones, determined by ELISA screening of the conditioned cell medium at multiple dilutions, were selected and used in a modified Simoa assay with two different detector beads (Nb-5, which is the C5 nanobody clone described above, or 4H1 (Sigma-Aldrich)). The ten best clones (62H12, 64C6, 33A8, 61A11, 36D12, 34H7, 50E9, 34C5, 55A6, and 42D10) all yielded up to 5-fold higher signal-to-noise than our best existing reagent, Ab6, (FIG. 13A). The sequences of the variable region heavy and light chains for each of the ten antibodies are provided in Table D and FIGS. 20A-J.

These antibodies were initially screened using SIMOA on recombinant ORF1p protein in buffer (Sample Diluent from Quanterix Corp. with added Triton-X 100) to identify pairs that provided improved sensitivity and specificity. Table 3 shows representative limits of detection (LODs) of select pairs of antibodies in diluent buffer. The LOD for the original Nb5/Ab6 pair was on average about 0.05 pg/ml.

TABLE 3
Representative LODs of select pairs of capture/detector antibodies
LOD (pg/ml)

| | | DETECTOR | | | | | | | | |
|---|---|---|---|---|---|---|---|---|---|---|
| | | | Nb5- | | | | | | | |
| | | Ab6 | 5LL | 33A8 | 34C5 | 34H7 | 64C6 | 61A11 | 62H12 | 55A6 |
| CAPTURE | 33A8 | 0.016 | | | | | | | | |
| | 34C5 | 0.026 | | | | 0.032 | | | | |
| | 34H7 | 0.006 | 0.043 | | | | | | | |
| | 55A6 | 0.026 | 0.055 | | | | | | | |
| | 64C6 | 0.034 | 0.041 | | | | | | | |
| | 36D12 | 0.046 | 0.050 | | | | | | | |
| | 42D10 | 0.021 | 0.020 | | | | | | | |
| | 61A11 | 0.042 | 0.066 | | | | | | | |
| | 62H12 | 0.008 | 0.062 | | | | | | | |
| | Nb5-5 | 0.050 | | 0.133 | 0.022 | 0.047 | 0.038 | 0.042 | 0.120 | |
| | 4H1 | 0.030 | 0.056 | 0.057 | | 0.044 | | | | 0.036 |
| | Ab6 | 0.102 | 0.044 | 0.063 | | | | | 0.113 | |

Signal/background was also determined for a number of the pairs by Simoa using recombinant ORF1p protein in diluent buffer; the results are shown in FIG. 15. When the rabbit monoclonal antibodies were used as capture and detector, the background was high. The best combinations were the rabbit monoclonal antibodies as capture/Ab6 detector; Nb5-5 capture/rabbit monoclonal antibodies detector; and 4H1 capture/rabbit monoclonal antibody detector pairs.

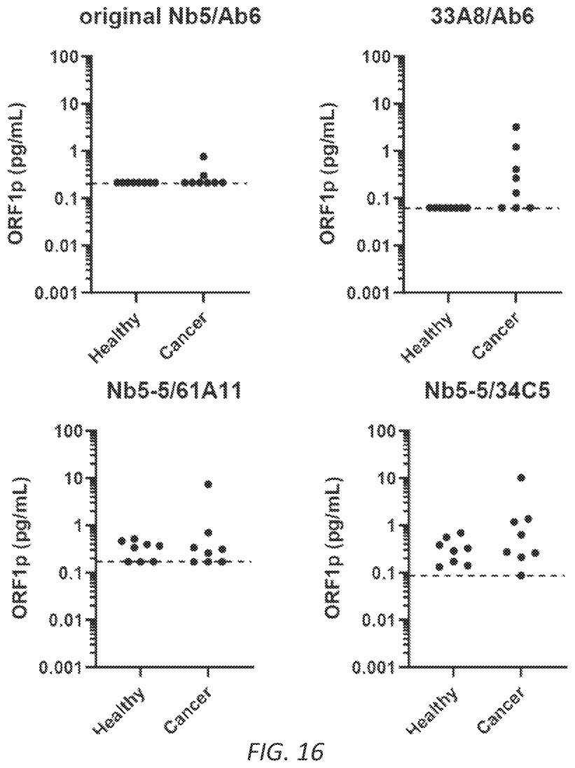

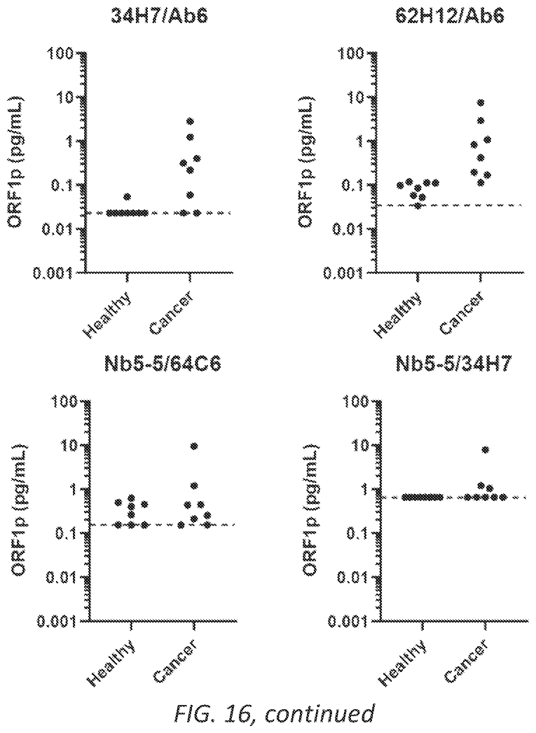

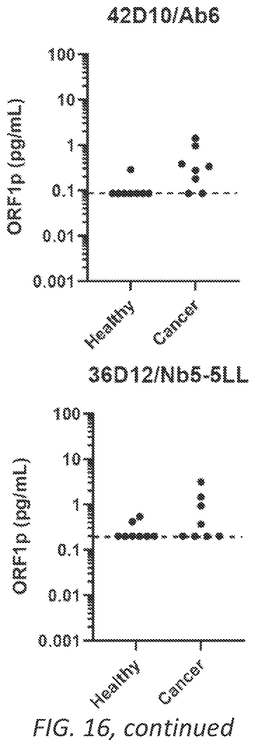

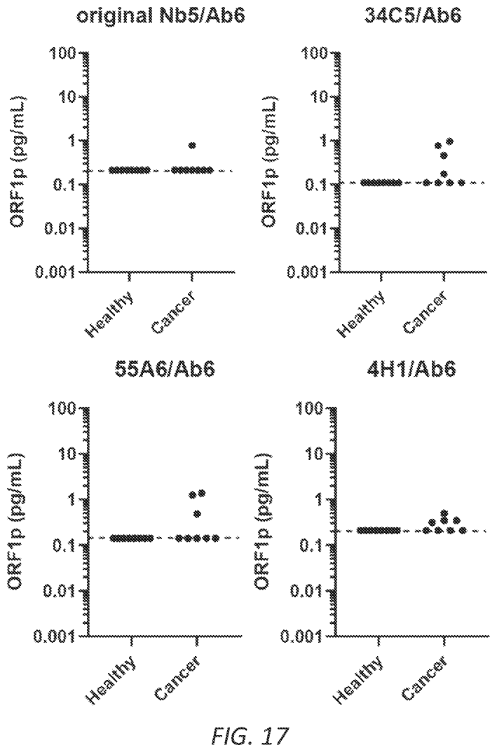

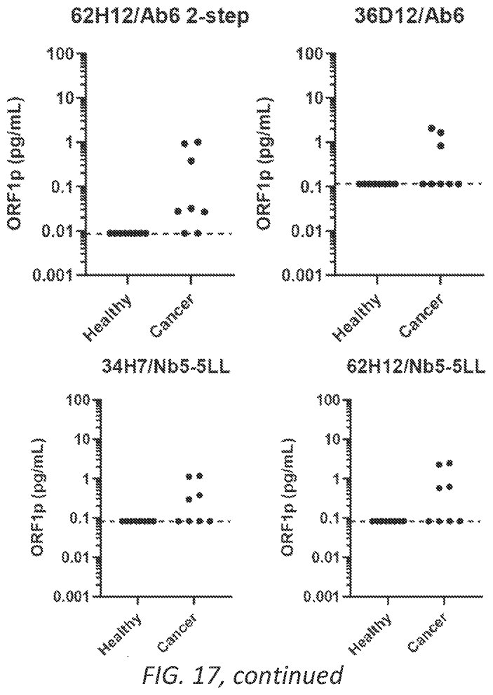

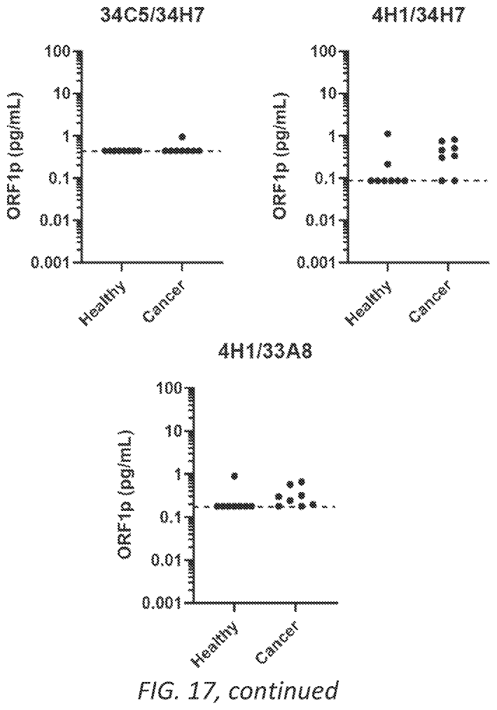

A selected number of pairs, including the original Nb5/Ab6 for comparison, were used to test ORF1p levels in plasma samples that were diluted four-fold in sample diluent (Quanterix Corp.) with Triton-X 100. The results are shown in FIGS. 16 and 17. In these assays, the original Nb5/Ab6 samples were diluted in Sample Diluent buffer, and the new assays used samples diluted in Sample Diluent+1% Triton-X. Sample Diluent was made using 20 g BSA (Millipore #820451); 100 mL 10×PBS (Sigma #P5493-1L); 10 mL 10% Tween-20 (Sigma #P9416-50ML); 500 uL Proclin 300 (Sigma 48912-U); 10 mL 0.5M EDTA (Sigma #E7889-100ML), made up to 1 L with MilliQ Water.

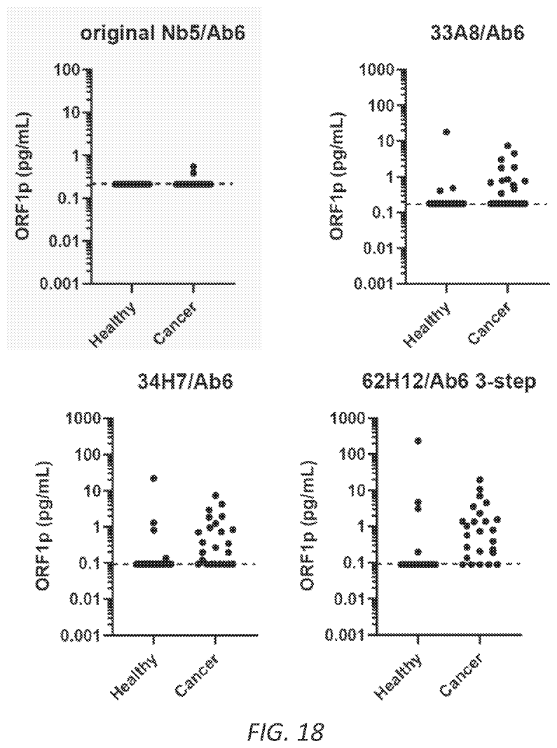

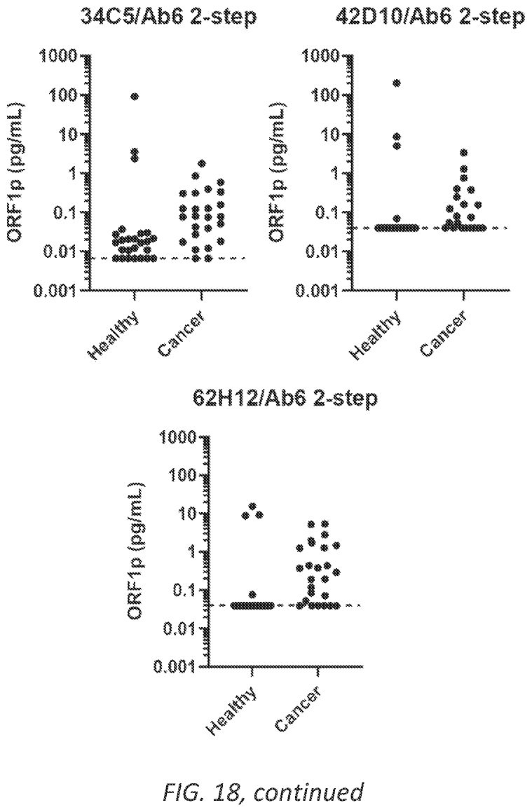

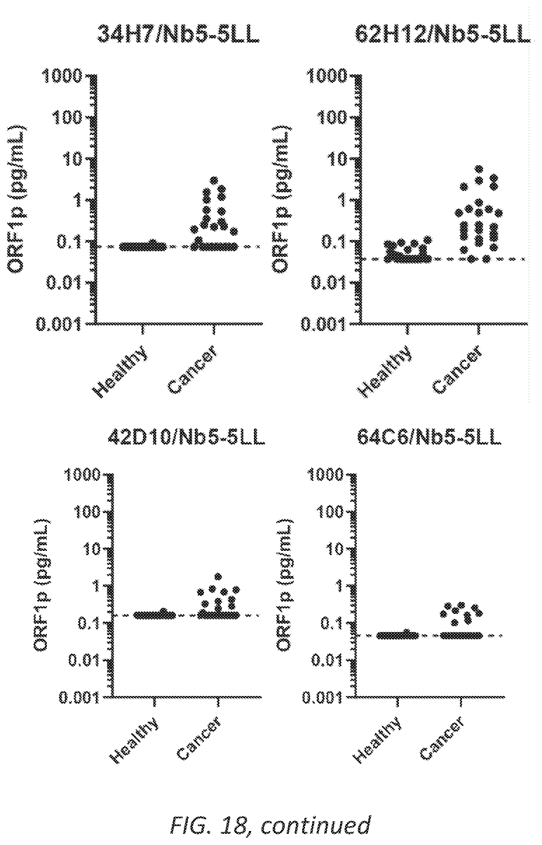

All assays were performed as three-step assays (separate capture, detector labeling, and streptavidin beta-galactosidase labeling steps), unless otherwise noted as two-step assays (combined capture and detector labeling steps). The results showed the variability in sensitivity depending on choice of capture/detector. FIG. 18 shows the result of assays performed in a cohort of 25 healthy subjects and 25 patients with breast, colorectal, or esophageal cancer.

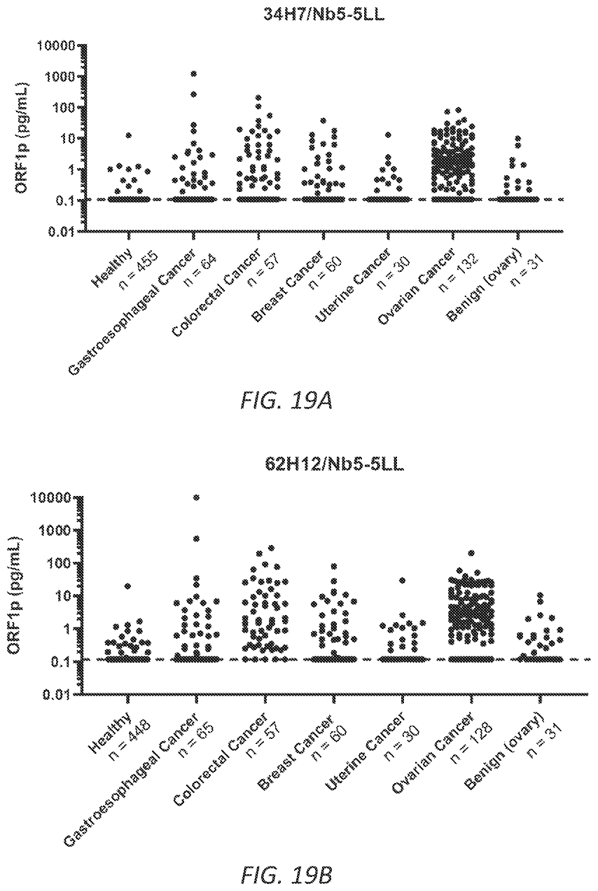

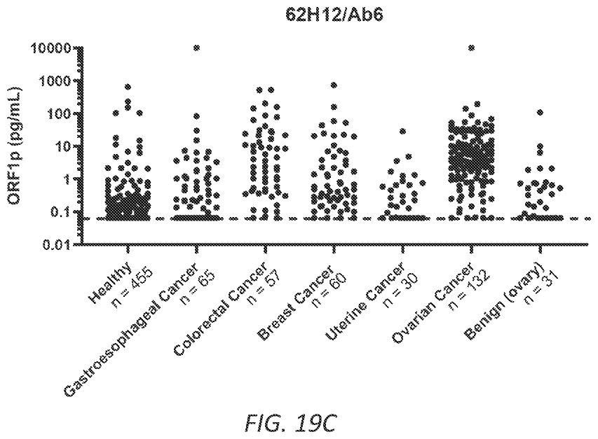

Selected pairs were then evaluated in a panel of cancers. The results are shown in FIGS. 19A-C.

TABLE D
Rabbit Anti-ORF1p monoclonal antibody variable region sequences

| | |
|---|---|
| >62H12-1 | METGLRWLLLVAVLKGVQCQSVKESGGRLVTPGTPLTLIC |
| Heavy | TVSGFSLSRYAVGWVRQAPGKGLEWIGYIWSGGYTDYAN |
| | WAKGRFTISKTSTTVDLKITSPTTEDTATYFCAREGDSDDYI |
| | YLNLWGPGTLVTVSS (SEQ ID NO:58) |
| >62H12-1 | MDTRAPTQLLGLLLLWLPGARCALVMTQTPSSVSAAVGGT |
| Light | VTINCQASESISSYLAWYQQKSGQPPKLLIYRASTLASGVPS |
| | RFKGSGSGTEFTLTIGDLECADAATYYCQQGYTSFNVDKA |
| | FGGGTEVVVK (SEQ ID NO:59) |
| >64C6-1 | METGLRWLLLLAALQGVQWRSVEESGGRLVTPGTPLKLPC |
| Heavy | ALSGFSLIIFAMIWVRLAPPKGLEWIGGIGINGNACYGRWA |
| | KGRFTISQTSSTTVDLKMTNLNTDDTATYFCVLRGFNLWG |
| | LGTLITVSS (SEQ ID NO:60) |
| >64C6-1 | MDTRAPTQLLGLLLLWLPGGICDPVMTQTPSSTSAAVGGT |
| Light | VTINCQSSQTVYKNWLSWFQQKPGQPPKLLIYDVSKLESG |
| | VPSRFKGSGSGTQFTLTISGVQCDDAATYYCAGGYSGKIFA |
| | FGGGTEVVVK (SEQ ID NO:61) |
| >33A8-1 | METGLRWLLLVAVLKGVQCQSLEESGGGLVQPEGSLTLTC |
| Heavy | KASGFSFINNNHICWVRQAPGKGLEWIACIYAGSTGYTYY |
| | ANWAKGRFTISKTSSTTVTLQMTSLTAADTATYFCARVPY |
| | AGYVNYGYTFFNLWGPGTLVTVSS (SEQ ID NO:62) |
| >33A8-1 | MDTRAPTQLLGLLLLWLPGATFAAVLTQTPSPVSAPVGGT |
| Light | VTISCQSSQSVYRTNDLAWYQQKPGQPPKLLIYWASKLAA |
| | GVPSRFKGSGSGTQFTLTISGVQCDDAATYYCLGGYYDDG |
| | DLNAFGGGTEVVVN (SEQ ID NO:63) |
| >61A11-1 | METGLRWLLLVAVLKGVQCQSVEESGGRLVTPGTPLTLTC |
| Heavy | TVSGIDLSSYVMGWVRQAPGKGLDWIGMIDTHDDTYFAP |
| | WARGRFTISRTSSTTVDLKVASTTTEDTATYFCAGNLALW |
| | GRGTLVTVSS (SEQ ID NO:64) |
| >61A11-1 | MDTRAPTQLLGLLLLWLPGATFAQVLTQTPSPVSVAVGGT |
| Light | VTISCQSSQSVSSDNWLGWYQQKPGQPPKLLIYRASKLASG |
| | VSSRFKGSGSGTQFILTISDLECDDAATYYCAGGYSGSIIAF |
| | GGGTEVVVK (SEQ ID NO:65) |
| >36D12-1 | METGLRWLLLVAVLKGVQCQSVEESGGRLVTPGTPLTLTC |
| Heavy | TVSGFSLSTYAMGWFRQAPGKGLEWIGTIWSGAFTDYAN |
| | WVNGRFTISKTSTTVDLKITSPTTEDTATYFCAREADSDLY |
| | TYFKLWGPGTLVTVSS (SEQ ID NO:66) |
| >36D12-1 | MDTRAPTQLLGLLLLWLPGARCAYDMTQTPASVEVAVGG |
| Light | TVTLKCQASESIEIYLAWYQQKPGQPPKLLVYDASTLASGV |
| | PSRFKGSGSGTQFTLTISGVECADAATYYCQQGYRSDNVH |
| | NVFGGGTEVVVK (SEQ ID NO:67) |
| >34H7-1 | METGLRWLLLVAVLKGVQCQSLEESGGDLVKPGASLTLTC |
| Heavy | TASGFSLNNYVMYWVRQAPGKGLESIACIYTGSGTTYYAT |
| | WAKGRFTISKTSSTTVTLQMTSLTAADTATYFCARRTYTSD |
| | SYAGSFNLWGPGTLVTVSS (SEQ ID NO:68) |
| >34H7-1 | MDTRAPTQLLGLLLLWLPGARCAYDMTQTPASVSEPVGGT |
| Light | VTIKCQASQSIGSNLAWYQQKPGQRPKLLIYAASYLASGAP |
| | SRFKGSGSGTEFTLTISGVQCDDAATYYCQQGWSSSDVDST |
| | FGGGTEVVVK (SEQ ID NO:69) |
| >50E9-2 | METGLRWLLLVAVFKGVQCQSVEESGGRLVTPGTPLTLTC |
| Heavy | TVSGFSLSRHYMSWVRQAPGKGLEWIGIIYDDDNRDHASW |
| | AKGRFTISRTSTTVDLKITSPTTEDTATYFCARVYASYGYAL |
| | NLWGQGTLVIVSS (SEQ ID NO:70) |
| >50E9-2 | MDTRAPTQLLGLLLLWLPGARCALVMTQTPPSVSAAVGGT |
| Light | VTINCQASESITNWLAWYQQKPGQPPKLLIYDASKLASGVS |
| | SRFSGSGSGTQFTLTISRVQCDDAATYYCQQGYTWNNIDN |
| | VFGGGTEVVVK (SEQ ID NO:71) |
| >34C5-1 | METGLRWLLLVAVLKGVQCQSVEESGGRLVKPDESLTLTC |
| Heavy | TVSGFSLSAYEMGWVRQAPGKGLEWIAAISTGDTPYYATW |
| | TKGRFTISRASSTTVDLKMTSPTTEDTATYFCARYGHNYEY |
| | VFDLWGQGTLVTVSS (SEQ ID NO:72) |
| >34C5-1 | MDTRAPTQLLGLLLLWLPGARCAYDMTQTPASVSAAVGG |
| Light | TVTINCQASQSIGSYLSWYQQKPGQPPKLLIYSASTLASGVP |
| | SRFSGSGYGTEFTLTIGGVQCDDAAAYYCQQGESIGSVHNV |
| | FGGGTEVVVA (SEQ ID NO:73) |
| >55A6-1 | METGLRWLLLVAVLKGVQCQSLEESGGDLVKPGASLTLTC |
| Heavy | TASGFSFINNNHICWVRQAPGKGLEWIACIYAGSNGYTYYT |
| | NWAKGRFTISKTSSTTVTLQMTSLTAADTATYFCARVPYA |
| | GYVNYGYAFFNLWGPGTLVTVSS (SEQ ID NO:74) |
| >55A6-1 | MDTRAPTQLLGLLLLWLPGATFAAVVTQTPSPVSAPVGGT |
| Light | VTISCQSSQSVYRINDLAWYQQKPGQPPKLLIYWASKLAIG |
| | VPSRFKGSGSGTQFTLTISGVQCDDAATYYCLGGYDDDGD |
| | LNTFGGGTEVVVN (SEQ ID NO:75) |
| >42D10-1 | METGLRWLLLVAVLKGVQCQSVEESGGRLVTPGTPLTLTC |
| Heavy | TVSGFSLSSYAMTWVRQAPGKGLEWIGYIYAGGIADYAN |
| | WAKGRFTISKTSTTVDLTITSPTTEDTATYFCAREGDSDDY |
| | VYLSLWGPGTLVTVSS (SEQ ID NO:76) |
| >42D10-1 | MDTRAPTQLLGLLLLWLPGARCALVMTQTPSSVSAAVGGT |
| Light | VTINCQASQSIVGYLAWYQQKPGQPPKLLIYRASTLASGVS |
| | SRFKGSGSGTEFTLTISGVQCADAATYYCQQGYINSNIDNT |
| | FGGGTEVVVK (SEQ ID NO:77) |

### Example 5. Development of Third Generation Assays

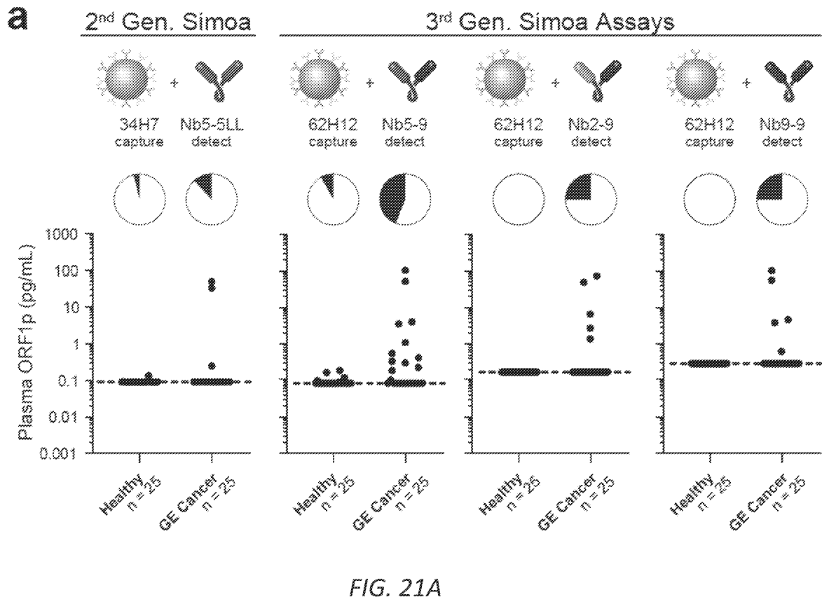

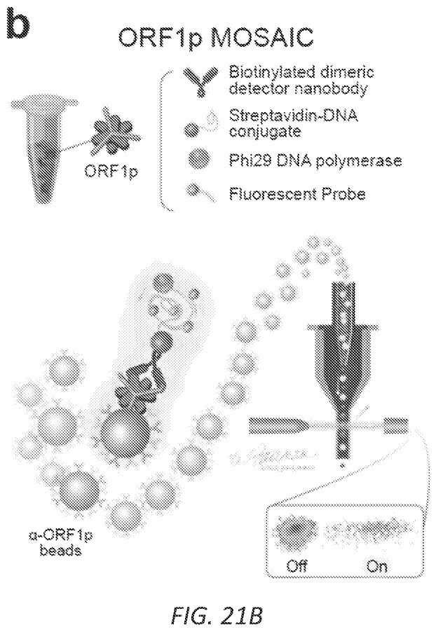

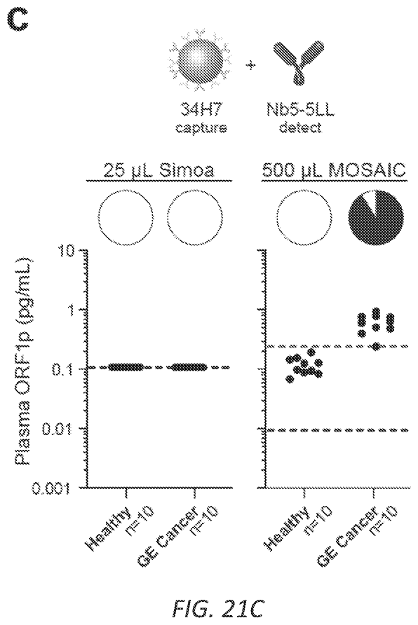

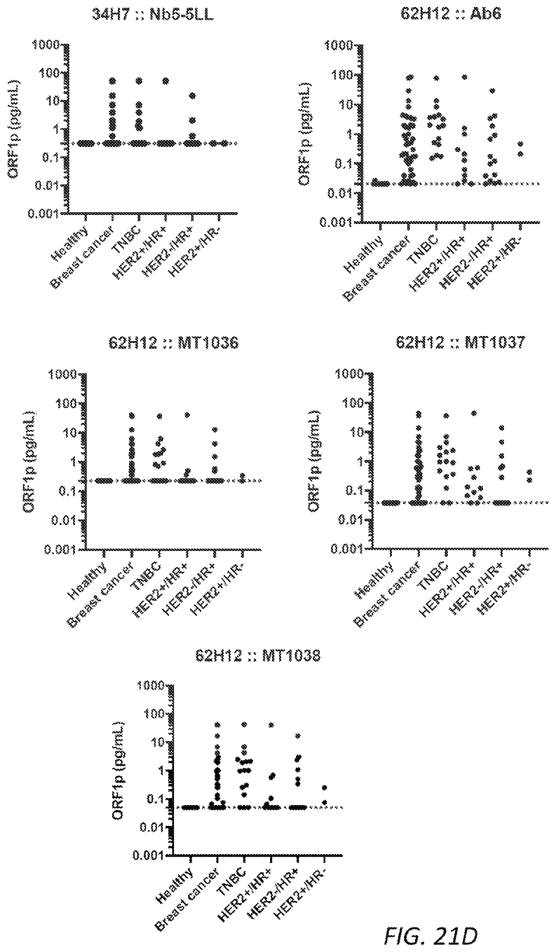

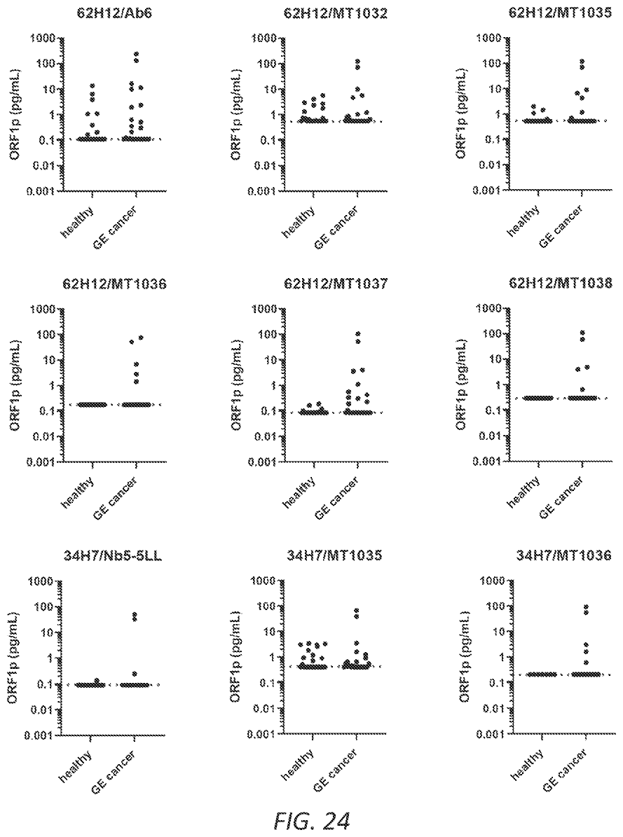

Building on the improvements made through nanobody engineering in our second-generation assays, we developed an expanded set of nanobody concatemers, including homodimeric, heterodimeric, and heterotrimeric anti-ORF1p nanobodies, and screened them in combination with 34H7 and 62H12 capture antibodies, resulting in “third-generation” assays (FIGS. 22A-B, 23, 24). We noticed that reagents containing Nb2 performed very well in SPR but poorly in Simoa detection, and we hypothesized this was because Nb2 contains a lysine in the CDR, which would be biotinylated in the procedure, reducing affinity. We therefore engineered the new reagents to be C-terminally biotinylated on cysteine residues and varied linker sequence. Five of these assays, which utilize Nb2- and Nb9-containing constructs, outperform our second-generation assays in a cohort of 25 GE cancer patients with ORF1p measurements that were mostly undetectable previously, while maintaining high specificity versus healthy individuals (FIG. 21A, FIG. 24).

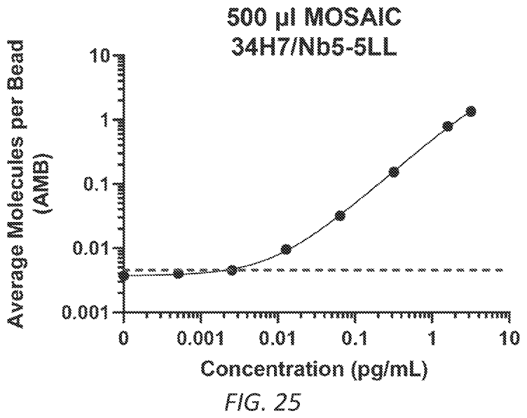

To leverage more sensitive assays for ORF1p detection, we next tested ORF1p affinity reagents from one of the second-generation Simoa assays on our recently developed Molecular On-bead Signal Amplification for Individual Counting platform (MOSAIC, FIG. 21B). MOSAIC develops localized on-bead signal from single captured molecules, in contrast to the microwell array format in Simoa, and improves analytical sensitivity by an order of magnitude over Simoa via increasing the number of beads counted(26). Furthermore, as the developed Simoa assays used only 25 μL plasma, we hypothesized that using larger plasma volumes would enhance ORF1p detectability by increasing the number of analyte molecules present. By using a 20-fold higher sample volume (500 μL plasma) and the MOSAIC platform, we achieved ten-fold higher analytical sensitivity, with a limit of detection of 0.002 μg/ml ORF1p (17 aM trimer, FIG. 25). Indeed, in a pilot cohort of gastroesophageal cancer and healthy patients, ORF1p levels in nine of ten previously undetectable cancer patients were readily discriminated from healthy individuals (FIG. 21C). Similar results were seen in a breast cancer cohort (FIG. 21D). Thus, in addition to improved affinity reagents, using larger sample volumes and more analytically sensitive technologies can further enhance both sensitivity and discrimination of circulating ORF1p levels between healthy controls and patients with cancer. The relative contributions of increased volume and the improved assay platform to the increased sensitivity remain to be explored; assay background seen in patient plasma but not in buffer can also be further optimized.

### REFERENCES

1. Bowtell D D, Böhm S, Ahmed A A, Aspuria P-J, Bast R C, Beral V, Berek J S, Birrer M J, Blagden S, Bookman M A, Brenton J D, Chiappinelli K B, Martins F C, Coukos G, Drapkin R, Edmondson R, Fotopoulou C, Gabra H, Galon J, Gourley C, Heong V, Huntsman D G, Iwanicki M, Karlan B Y, Kaye A, Lengyel E, Levine D A, Lu K H, McNeish I A, Menon U, Narod S A, Nelson B H, Nephew K P, Pharoah P, Powell D J, Ramos P, Romero I L, Scott C L, Sood A K, Stronach E A, Balkwill F R. Rethinking ovarian cancer II: reducing mortality from high-grade serous ovarian cancer. Nature Reviews Cancer. 2015; 15(11):668-79. doi: 10.1038/nrc4019.
2. Torre L A, Trabert B, DeSantis C E, Miller K_{D}, Samimi G, Runowicz C D, Gaudet M M, Jemal A, Siegel R L. Ovarian cancer statistics, 2018. CA: A Cancer Journal for Clinicians. 2018; 68(4):284-96. doi.org/10.3322/caac.21456.
3. Cescon D W, Bratman S V, Chan S M, Siu L L. Circulating tumor DNA and liquid biopsy in oncology. Nature Cancer. 2020; 1(3):276-90. doi: 10.1038/s43018-020-0043-5.
4. Elias K M, Fendler W, Stawiski K, Fiascone S J, Vitonis A F, Berkowitz R S, Frendl G, Konstantinopoulos P, Crum C P, Kedzierska M, Cramer D W, Chowdhury D. Diagnostic potential for a serum miRNA neural network for detection of ovarian cancer. eLife. 2017; 6:e28932. doi: 10.7554/eLife.28932.
5. Dochez V, Caillon H, Vaucel E, Dimet J, Winer N, Ducarme G. Biomarkers and algorithms for diagnosis of ovarian cancer: CA125, HE4, RMI and ROMA, a review. Journal of Ovarian Research. 2019; 12(1):28. doi: 10.1186/s13048-019-0503-7.
6. Drapkin R, von Horsten H H, Lin Y, Mok S C, Crum C P, Welch W R, Hecht J L. Human Epididymis Protein 4 (HE4) Is a Secreted Glycoprotein that Is Overexpressed by Serous and Endometrioid Ovarian Carcinomas. Cancer Research. 2005; 65(6):2162. doi: 10.1158/0008-5472.CAN-04-3924.
7. Burns K H. Transposable elements in cancer. Nature Reviews Cancer. 2017; 17(7):415-24. doi: 10.1038/nrc.2017.35.
8. Rodić N, Sharma R, Sharma R, Zampella J, Dai L, Taylor M S, Hruban R H, Iacobuzio-Donahue C A, Maitra A, Torbenson M S, Goggins M, Shih I-M, Duffield A S, Montgomery E A, Gabrielson E, Netto G J, Lotan T L, De Marzo A M, Westra W, Binder Z A, Orr B A, Gallia G L, Eberhart C G, Boeke J D, Harris C R, Burns K H. Long Interspersed Element-1 Protein Expression Is a Hallmark of Many Human Cancers. The American Journal of Pathology. 2014; 184(5):1280-6. doi: 10.1016/j.ajpath.2014.01.007.
9. Pisanic T R, Asaka S, Lin S-F, Yen T-T, Sun H, Bahadirli-Talbott A, Wang T-H, Burns K H, Wang T-L, Shih I-M. Long Interspersed Nuclear Element 1 Retrotransposons Become Deregulated during the Development of Ovarian Cancer Precursor Lesions. The American Journal of Pathology. 2019; 189(3):513-20. doi.org/I0.1016/j.ajpath.2018.11.005.
10. Xia Z, Cochrane D R, Tessier-Cloutier B, Leung S, Karnezis A N, Cheng A S, Farnell D A, Magrill J, Schmidt D, Kommoss S, Kommoss F K F, Kommoss F, McAlpine J N, Gilks C B, Koebel M, Rabban J T, Huntsman D G. Expression of L1 retrotransposon open reading frame protein 1 in gynecologic cancers. Human Pathology. 2019; 92:39-47. doi.org/10.1016/j.humpath.2019.06.001.
11. Rissin D M, Kan C W, Campbell T G, Howes S C, Fournier D R, Song L, Piech T, Patel P P, Chang L, Rivnak A J, Ferrell E P, Randall J D, Provuncher G K, Walt D R, Duffy D C. Single-molecule enzyme-linked immunosorbent assay detects serum proteins at subfemtomolar concentrations. Nature Biotechnology. 2010; 28(6):595-9. doi: 10.1038/nbt.1641.
12. Ignatiadis M, Sledge G W, Jeffrey S S. Liquid biopsy enters the clinic implementation issues and future challenges. Nature Reviews Clinical Oncology. 2021; 18(5):297-312. doi: 10.1038/s41571-020-00457-x.
13. Bettegowda C, Sausen M, Leary R J, Kinde I, Wang Y, Agrawal N, Bartlett B R, Wang H, Luber B, Alani R M, Antonarakis E S, Azad N S, Bardelli A, Brem H, Cameron J L, Lee C C, Fecher L A, Gallia G L, Gibbs P, Le D, Giuntoli R L, Goggins M, Hogarty M D, Holdhoff M, Hong S-M, Jiao Y, Juhl H H, Kim J J, Siravegna G, Laheru D A, Lauricella C, Lim M, Lipson E J, Marie S K N, Netto G J, Oliner K S, Olivi A, Olsson L, Riggins G J, Sartore-Bianchi A, Schmidt K, Shih 1-M, Oba-Shinjo S M, Siena S, Theodorescu D, Tie J, Harkins T T, Veronese S, Wang T-L, Weingart J D, Wolfgang C L, Wood L D, Xing D, Hruban R H, Wu J, Allen P J, Schmidt C M, Choti M A, Velculescu V E, Kinzler K W, Vogelstein B, Papadopoulos N, Diaz L A. Detection of Circulating Tumor DNA in Early- and Late-Stage Human Malignancies. Science Translational Medicine. 2014; 6(224):224ra24. doi: 10.1126/scitranslmed.3007094.
14. Abbosh C, Swanton C, Birkbak N J. Clonal haematopoiesis: a source of biological noise in cell-free DNA analyses. Annals of Oncology. 2019; 30(3):358-9. doi: 10.1093/annonc/mdy552.
15. Serrano M J, Garrido-Navas M C, Diaz Mochon J J, Cristofanilli M, Gil-Bazo I, Pauwels P, Malapelle U, Russo A, Lorente J A, Ruiz-Rodriguez A J, Paz-Ares L G, Vilar E, Raez L E, Cardona A F, Rolfo C. Precision Prevention and Cancer Interception: The New Challenges of Liquid Biopsy. Cancer Discovery. 2020; 10(11):1635. doi: 10.1158/2159-8290.CD-20-0466.
16. Welsh J B, Sapinoso L M, Kern S G, Brown D A, Liu T, Bauskin A R, Ward R L, Hawkins N J, Quinn D I, Russell P J, Sutherland R L, Breit S N, Moskaluk C A, Frierson H F, Hampton G M. Large-scale delineation of secreted protein biomarkers overexpressed in cancer tissue and serum. Proceedings of the National Academy of Sciences. 2003; 100(6):3410. doi: 10.1073/pnas.0530278100.
17. Grundy E E, Diab N, Chiappinelli K B. Transposable element regulation and expression in cancer. The FEBS Journal. 2021; n/a(n/a). doi.org/10.1111/febs.15722.
18. Rodriguez-Martin et al., Pan-cancer analysis of whole genomes identifies driver rearrangements promoted by LINE-1 retrotransposition. Nature Genetics. 2020; 52(3):306-19. doi: 10.1038/s41588-019-0562-0.
19. Scott E C, Gardner E J, Masood A, Chuang N T, Vertino P M, Devine S E. A hot L1 retrotransposon evades somatic repression and initiates human colorectal cancer. Genome Research. 2016; 26(6):745-55.
20. Rodid N, Steranka J P, Makohon-Moore A, Moyer A, Shen P, Sharma R, Kohutek Z A, Huang C R, Ahn D, Mita P, Taylor M S, Barker N J, Hruban R H, Iacobuzio-Donahue C A, Boeke J D, Burns K H. Retrotransposon insertions in the clonal evolution of pancreatic ductal adenocarcinoma. Nature Medicine. 2015; 21(9):1060-4. doi: 10.1038/nm.3919.
21. Goodier J L. Restricting retrotransposons: a review. Mobile DNA. 2016; 7(1):16. doi: 10.1186/s13100-016-0070-z.
22. Ardeljan D, Taylor M S, Ting D T, Burns K H. The Human Long Interspersed Element-1 Retrotransposon: An Emerging Biomarker of Neoplasia. Clinical Chemistry. 2017; 63(4):816-22. doi: 10.1373/clinchem.2016.257444.
23. Pavlou M P, Diamandis E P, Blasutig I M. The Long Journey of Cancer Biomarkers from the Bench to the Clinic. Clinical Chemistry. 2013; 59(1):147-57. doi: 10.1373/clinchem.2012.184614.
24. Enroth S, Johansson Å, Enroth S B, Gyllensten U. Strong effects of genetic and lifestyle factors on biomarker variation and use of personalized cutoffs. Nature Communications. 2014; 5(1):4684. doi: 10.1038/ncomms5684.
25. Wu D, Dinh T L, Bausk B P, Walt D R. Long-Term Measurements of Human Inflammatory Cytokines Reveal Complex Baseline Variations between Individuals. The American Journal of Pathology. 2017; 187(12):2620-6. doi: 10.1016/j.ajpath.2017.08.007.
26. Cohen L, Cui N, Cai Y, Garden P M, Li X, Weitz D A, Walt D R. Single Molecule Protein Detection with Attomolar Sensitivity Using Droplet Digital Enzyme-Linked Immunosorbent Assay. ACS Nano. 2020; 14(8):9491-501. doi: 10.1021/acsnano.0c02378.
27. Ardeljan D, Wang X, Oghbaie M, Taylor M S, Husband D, Deshpande V, Steranka J P, Gorbounov M, Yang W R, Sie B, Larman H B, Jiang H, Molloy K R, Altukhov I, Li Z, McKerrow W, Fenyo D, Burns K H, LaCava J. LINE-1 ORF2p expression is nearly imperceptible in human cancers. Mobile DNA. 2019; 11(1):1. doi: 10.1186/s13100-019-0191-2.
28. Wellner A, McMahon C, Gilman M S A, Clements J R, Clark S, Nguyen K M, Ho M H, Hu V J, Shin J-E, Feldman J, Hauser B M, Caradonna T M, Wingler L M, Schmidt A G, Marks D S, Abraham J, Kruse A C, Liu C C. Rapid generation of potent antibodies by autonomous hypermutation in yeast. Nature Chemical Biology. 2021; 17(10):1057-64. doi: 10.1038/s41589-021-00832-4.
29. Zupancic J M, Desai A A, Schardt J S, Pornnoppadol G, Makowski E K, Smith M D, Kennedy A A, Garcia de Mattos Barbosa M, Cascalho M, Lanigan T M, Tai A W, Tessier P M. Directed evolution of potent neutralizing nanobodies against SARS-CoV-2 using CDR-swapping mutagenesis. Cell Chemical Biology. 2021; 28(9):1379-88.e7. doi: doi.org/10.1016/j.chembiol.2021.05.019.
30. Wu C, Dougan T J, Walt D R. MOSAIC: High-Throughput, High-Multiplex Digital Protein Detection with Attomolar Sensitivity. Wu, C., Dougan, T. J. & Walt, D. R. High-Throughput, High-Multiplex Digital Protein Detection with Attomolar Sensitivity. ACS Nano, doi: 10.1021/acsnano.1c08675 (2022).
31. Sudmant P H, et al., An integrated map of structural variation in 2,504 human genomes. Nature. 2015; 526(7571):75-81. doi: 10.1038/nature15394.
32. Mahmood W, Erichsen L, Ott P, Schulz W A, Fischer J C, Arauzo-Bravo M J, Bendhack M L, Hassan M, Santourlidis S. Aging-associated distinctive DNA methylation changes of LINE-1 retrotransposons in pure cell-free DNA from human blood. Scientific Reports. 2020; 10(1):22127. doi: 10.1038/s41598-020-79126-z.
33. Crow M K. Long interspersed nuclear elements (LINE-1): Potential triggers of systemic autoimmune disease. Autoimmunity. 2010; 43(1):7-16. doi: 10.3109/08916930903374865.
34. Mavragani C P, Sagalovskiy I, Guo Q, Nezos A, Kapsogeorgou E K, Lu P, Liang Zhou J, Kirou K A, Seshan S V, Moutsopoulos H M, Crow M K. Expression of Long Interspersed Nuclear Element 1 Retroelements and Induction of Type I Interferon in Patients With Systemic Autoimmune Disease. Arthritis & Rheumatology. 2016; 68(11):2686-96. doi.org/10.1002/art.39795.
35. Carter V, LaCava J, Taylor M S, Liang S Y, Mustelin C, Ukadike K C, Bengtsson A, Lood C, Mustelin T. High Prevalence and Disease Correlation of Autoantibodies Against p40 Encoded by Long Interspersed Nuclear Elements in Systemic Lupus Erythematosus. Arthritis & Rheumatology. 2020; 72(1):89-99. doi.org/10.1002/art.41054.
36. Ukadike K C, Ni K, Wang X, Taylor M S, LaCava J, Pachman L M, Eckert M, Stevens A, Lood C, Mustelin T. IgG and IgA autoantibodies against L1 ORF1p expressed in granulocytes correlate with granulocyte consumption and disease activity in pediatric systemic lupus erythematosus. Arthritis Research & Therapy. 2021; 23(1):153. doi: 10.1186/s13075-021-02538-3.
37. Crow M K. Reactivity of IgG With the p40 Protein Encoded by the Long Interspersed Nuclear Element 1 Retroelement: Comment on the Article by Carter et al. Arthritis Rheumatol. 2020; 72(2):374-6. Epub 2019/12/27. doi: 10.1002/art.41102.
38. Sharma R, Rodić N, Burns K H, Taylor M S. Immunodetection of Human LINE-1 Expression in Cultured Cells and Human Tissues. Methods Mol Biol. 2016; 1400:261-80. doi: 10.1007/978-1-4939-3372-3_17.
39. Cohen L and Walt D R. Highly Sensitive and Multiplexed Protein Measurements. Chem. Rev. 2019, 119, 293-321.
40. D. Wild, The Immunoassay Handbook: Theory and applications of ligand binding, ELISA and related techniques (Elsevier, Amsterdam, The Netherlands, ed. 4th, 2013; linkinghub.elsevier.com/retrieve/pii/B9781455778966000583).
41. Cohen, L. et al., Single Molecule Arrays for ultra-sensitive detection of rat cytokines in serum, J. Immunol. Methods 452, 20-25 (2018).
42. Rissin D M, et al., Simultaneous detection of single molecules and singulated ensembles of molecules enables immunoassays with broad dynamic range, Anal. Chem. 83, 2279-2285 (2011).
43. Katz-Summercorn, A. C. et al. Multi-omic cross-sectional cohort study of pre-malignant Barrett's esophagus reveals early structural variation and retrotransposon activity. Nat Commun 13, 1407, doi:10.1038/s41467-022-28237-4 (2022).
44. Taylor, M. S. et al. Affinity proteomics reveals human host factors implicated in discrete stages of LINE-1 retrotransposition. Cell 155, 1034-1048, doi:10.1016/j.cell.2013.10.021 (2013).
45. Taylor, M. S. et al. Dissection of affinity captured LINE-1 macromolecular complexes. Elife 7, doi:10.7554/eLife.30094 (2018).
46. De Cecco, M. et al. L1 drives IFN in senescent cells and promotes age-associated inflammation. Nature 566, 73-78, doi:10.1038/s41586-018-0784-9 (2019).
47 Simon, M. et al. LINE1 Derepression in Aged Wild-Type and SIRT6-Deficient Mice Drives Inflammation. Cell Metab 29, 871-885 e875, doi:10.1016/j.cmet.2019.02.014 (2019).
48. Rajurkar, M. et al. Reverse Transcriptase Inhibition Disrupts Repeat Element Life Cycle in Colorectal Cancer. Cancer Discov 12, 1462-1481, doi:10.1158/2159-8290.CD-21-1117 (2022).
49. Fridy, P. C. et al. A robust pipeline for rapid production of versatile nanobody repertoires. Nat Methods 11, 1253-1260, doi:10.1038/nmeth.3170 (2014).
50. Skates, S. J. et al. Early Detection of Ovarian Cancer using the Risk of Ovarian Cancer Algorithm with Frequent CA125 Testing in Women at Increased Familial Risk—Combined Results from Two Screening Trials. Clin Cancer Res 23, 3628-3637, doi:10.1158/1078-0432.CCR-15-2750 (2017).
51. Skates, S J et al., Screening based on the risk of cancer calculation from Bayesian hierarchical change-point and mixture models of longitudinal markers. J Am Stat Assoc 96(454), 429-439 (2001).
52. Mast, F D et al. Highly synergistic combinations of nanobodies that target SARS-CoV-2 and are resistant to escape. Elife 2021; 10 doi 10.7554/eLife.73027.
53. Wolters, J C et al. Translational Targeted Proteomics Profiling of Mitochondrial Energy Metabolic Pathways in Mouse and Human Samples. J Proteome Res 2016; 15(9):3204-13 doi 10.1021/acs.jproteome.6b00419.
54. Kaplan, E L and Meier, P. Nonparametric estimation from incomplete observations. Journal of the American statistical association 1958; 53(282):457-81.
55. Cox, DR. Regression models and life-tables. Journal of the Royal Statistical Society: Series B (Methodological) 1972; 34(2):187-202.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
providing a sample, preferably a sample comprising a biofluid or tissue, preferably wherein the biofluid is blood, serum, or plasma, or the tissue is a tissue lysate, from a subject, and determining a level of ORF1p in the sample with an ultrasensitive protein assay.

2. The method of claim 1, further comprising comparing the level of ORF1p to a disease reference, wherein a level of ORF1p above the reference indicates that the subject has or is at risk of developing cancer.

3. The method of claim 1, wherein the cancer is a carcinoma.

4. The method of claim 3, wherein the carcinoma is ovarian, breast, liver, colon/colorectal, lung, esophageal, prostate, gastric, head and neck, brain (optionally glioblastoma), soft tissue, kidney, gallbladder, bile duct (cholangiocarcinoma), bladder, uterine, or pancreatic cancer, blood or bone marrow (optionally lymphoma, leukemia, or myeloma), or skin cancer (optionally melanoma).

5. The method of claim 4, wherein the ovarian cancer is high-grade serous ovarian cancer (HGSOC).

6. The method of claim 1, wherein the ultrasensitive assay is Single-Molecule Arrays (SIMOA); Molecular On-bead Signal Amplification for Individual Counting (MOSAIC); Meso Scale Discovery (MSD); Single-Molecule Counting (SMC); nucleic acid linked immune-sandwich assay (NULISA); LUMINEX; SOMAscan Assays; NAB-SURE; mass spectrometry (optionally MALDI-MS), and/or mass cytometry (optionally CyTOF).

7. The method of claim 1, wherein determining a level of ORF1p comprises contacting the sample with a capture or detection reagent comprising a nanobody selected from Nb2, Nb5, Nb9, Nb10, or NB21 or an ORF1p-binding derivative comprising CDRs thereof, or a concatemer thereof, optionally MT1032, MT1033, MT1034, MT1035, MT1036, MT1037, MT1038, MT1039, or MT1040, and/or a monoclonal antibody selected from 62H12, 64C6, 33A8, 61A11, 36D12, 34H7, 50E9, 34C5, 55A6, 42D10, 4H1, Ab6, Ab54 or an antigen-binding fragment thereof, optionally wherein the capture/detection reagents are 62H12/MT1036, 62H12/MT1037, 62H12/MT1038, 62H12/Ab6, 34H7/Nb5-5LL, 34H7/Ab6, 62H12/Nb5-5LL, 4H1/Nb5-5, or 4H1/Nb5-5LL.

8. The method of claim 7, wherein the nanobody comprises a sequence at least 80%, 85%, 90%, or 95% identical to a sequence in Table B, preferably wherein the CDRs of the nanobody are identical to those from a sequence in Table B.

9. The method of claim 1, further comprising recommending or sending the subject for additional evaluation, optionally by imaging and/or biopsy.

10. The method of claim 1, further comprising administering a treatment for cancer to a subject who has been identified as having or at risk of developing cancer.

11. The method of claim 8, wherein the treatment comprises chemotherapy, hormone therapy, immunotherapy, radiation, or surgical resection.

12. The method of claim 1, further comprising determining a level of ORF1p in the subject after administration of the treatment, and comparing the level of ORF1p prior to treatment with the level of ORF1p during and/or after treatment, wherein a decrease in the level of ORF1p indicates that the treatment is effective in treating the cancer.

13. A single domain antibody or antigen-binding fragment thereof that binds to human ORF1p, comprising a sequence at least 90% identical to a nanobody sequence or CDR1, CDR2, and CDR3 therefrom as shown in table B.

14. The single domain antibody or antigen-binding fragment thereof of claim 13, comprising CDR1, CDR2, and CDR3 as shown in Table B.

15. A fusion construct comprising at least two, optionally three, four, or five, of the single domain antibodies or antigen-binding fragments thereof of claim 13, optionally with linkers therebetween, optionally as shown in Table C, optionally MT1032, MT1033, MT1034, MT1035, MT1036, MT1037, MT1038, MT1039, and MT1040.

16. An antibody or antigen binding portion thereof that specifically binds to human ORF1p, wherein the antibody or antigen binding portion thereof comprises at least one of:
a heavy chain variable region (VH) comprising or consisting of a VH sequence that is at least 95% identical to a sequence shown in Table D or FIGS. 20A-J, or CDR1, CDR2, and CDR3 therefrom; and/or
a light chain variable region (VL) comprising or consisting of a VL sequence that is at least 95% identical to a sequence shown in Table D or FIGS. 20A-J, or CDR1, CDR2, and CDR3 therefrom, preferably wherein the VH and VL or CDRs are from the same antibody.

17. The antibody or antigen binding portion thereof of claim 16, wherein the antibody comprises a constant region, optionally as shown in Table A.

18. The single domain antibody or antigen-binding fragment thereof of claim 13, which is fused to a tag.

19. The single-domain antibody or antigen-binding fragment thereof, the fusion construct, or the antibody or antigen binding portion thereof, of claim 18, wherein the tag is an oligonucleotide, peptide, chemiluminescent, fluorescent, radioactive, or colorimetric label.

20. The single-domain antibody or antigen-binding fragment thereof, the fusion construct, or the antibody or antigen binding portion thereof, of claim 19, wherein the radiolabel is ¹²⁵I.

21. A nucleic acid molecule encoding the single-domain antibody or antigen-binding fragment thereof, the fusion construct, or the antibody or antigen binding portion thereof, of claim 13.

22. A vector comprising the nucleic acid molecule according to claim 21, and optionally a promoter.

23. A host cell comprising the nucleic acid molecule according to claim 21, and optionally expressing the single-domain antibody or antigen-binding fragment thereof, the fusion construct, or the antibody or antigen binding portion thereof.


SEQUENCE LISTING (102 sequences)

SEQ ID NO: 1 - protein, 123 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVS

SEQ ID NO: 2 - protein, 8 aa, synthetic construct
GGTSSNYG

SEQ ID NO: 3 - protein, 8 aa, synthetic construct
ISWSGSRT

SEQ ID NO: 4 - protein, 17 aa, synthetic construct
TAVREYRDYPQRDNFDY

SEQ ID NO: 5 - protein, 5 aa, synthetic construct
  REGION 1..5: note = The entire sequence of amino acids 1-5 can be repeated 1-4 times
GGGGS

SEQ ID NO: 6 - protein, 5 aa, synthetic construct
GGGGS

SEQ ID NO: 7 - protein, 10 aa, synthetic construct
GGGGSGGGGS

SEQ ID NO: 8 - protein, 15 aa, synthetic construct
GGGGSGGGGSGGGGS

SEQ ID NO: 9 - protein, 20 aa, synthetic construct
GGGGSGGGGSGGGGSGGGGS

SEQ ID NO: 10 - protein, 5 aa, synthetic construct
  REGION 1..5: note = The entire sequence of amino acids 1-5 can be repeated 1-4 times
GGGGA

SEQ ID NO: 11 - protein, 5 aa, synthetic construct
  REGION 1..5: note = The entire sequence of amino acids 1-5 can be repeated 1-4 times
EAAAK

SEQ ID NO: 12 - protein, 5 aa, synthetic construct
  REGION 1..5: note = The entire sequence of amino acids 1-5 can be repeated 1-4 times
DAAAR

SEQ ID NO: 13 - protein, 56 aa, synthetic construct
GGGGSGGGGSGGGGSGGGGSEAAAKEAAAKEAAAKSGGGGSGGGGSGGGGSGGGGS

SEQ ID NO: 14 - protein, 330 aa, Homo sapiens [sequence omitted]

SEQ ID NO: 15 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 213 aa, Homo sapiens
LGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 107 aa, Homo sapiens [sequence omitted]

SEQ ID NO: 18 - protein, 324 aa, Mus musculus
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 19 - protein, 404 aa, Mus musculus
KTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGLDLDDICAEAKDGELDGLWTTITIFISLFLLSVCYSASVTLFKVKWIFSSVVELKQKISPDYRNMIGQGA

SEQ ID NO: 20 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 123 aa, synthetic construct
DVQLVESGGGLVQAGESLRLSCAASGRTLSSLNMAWFRQVPGKERDFVSLISRSGSTDYVHSVRGRFTISRDNAKNTVYLEMNSLKPEDTAVYYCAADDNYNGRYPFRDEYEYWGQGTQVTVS

SEQ ID NO: 22 - protein, 8 aa, synthetic construct
GRTLSSLN

SEQ ID NO: 23 - protein, 7 aa, synthetic construct
ISRSGST

SEQ ID NO: 24 - protein, 18 aa, synthetic construct
AADDNYNGRYPFRDEYEY

SEQ ID NO: 25 - protein, 128 aa, synthetic construct
DVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVS

SEQ ID NO: 26 - protein, 8 aa, synthetic construct
GRTDDIYT

SEQ ID NO: 27 - protein, 12 aa, synthetic construct
ISWSITRRSSIT

SEQ ID NO: 28 - protein, 18 aa, synthetic construct
AAKTGYSGSMSSYQDYDS

SEQ ID NO: 29 - protein, 126 aa, synthetic construct
QVHLVESGGKLVQAGESLKLSCEVSGRTFSISGAGWFRQAPGKEREFVAAITWTGGYPYYDASVKGRFTISREDPDNTVYLSMNSLKPEDTAVYYCATRRAGDLDFSYAQQGRYDNWGQGTQVTVS

SEQ ID NO: 30 - protein, 8 aa, synthetic construct
GRTFSISG

SEQ ID NO: 31 - protein, 8 aa, synthetic construct
ITWTGGYP

SEQ ID NO: 32 - protein, 20 aa, synthetic construct
ATRRAGDLDFSYAQQGRYDN

SEQ ID NO: 33 - protein, 126 aa, synthetic construct
QVQLVESGGGLVQAGESLRLSCQVSGRTFSISGAGWFRQAPGKEREFVAAITWTGGYPYYATSVKGRFTIARDDPENRVYLSMNSLKPEDTAVYYCATRRAGSLDFDYAQQTRYDNWGQGTQVTVS

SEQ ID NO: 34 - protein, 8 aa, synthetic construct
GRTFSISG

SEQ ID NO: 35 - protein, 8 aa, synthetic construct
ITWTGGYP

SEQ ID NO: 36 - protein, 20 aa, synthetic construct
ATRRAGSLDFDYAQQTRYDN

SEQ ID NO: 37 - protein, 123 aa, synthetic construct
QVQLAESGGGLVQAGGSLRLSCAASGRTSTIYIMGWFRQAPGKDREFVAGITYTGGYPYYADSVQGRFTLSNDNADHTAYLQMNNLKPEDSAVYYCAARSRGGITTHERDFGSWGQGTQVTVS

SEQ ID NO: 38 - protein, 8 aa, synthetic construct
GRTSTIYI

SEQ ID NO: 39 - protein, 8 aa, synthetic construct
ITYTGGYP

SEQ ID NO: 40 - protein, 17 aa, synthetic construct
AARSRGGITTHERDFGS

SEQ ID NO: 41 - DNA, 369 bp, synthetic construct
  source 1..369: mol_type = other DNA
caggtacagcttgtggaatcagggggtgaccttgtgcaggcaggagggtcactgcgcttatcttgtgcggtcagtgggggcacgtcatcaaactacgggatgggttggtttcgtcaagcccctggaaaggagcgcgagtttgtctcatcgatctcctggtcaggcagtcgtactttatatagcgactcagtgaaaggccgcttcacgattagtcgtgataatgcgaaaaacaccgttgacttgcagatgaactctttgaagcctgaagacacggcagtctattattgcaccgcagtacgcgagtatcgcgactacccgcagcgcgataactttgactattggggacaagggacgcaggttacggtaagt

SEQ ID NO: 42 - protein, 267 aa, synthetic construct
DVQLVESGGGLVQAGESLRLSCAASGRTLSSLNMAWFRQVPGKERDFVSLISRSGSTDYVHSVRGRFTISRDNAKNTVYLEMNSLKPEDTAVYYCAADDNYNGRYPFRDEYEYWGQGTQVTVSGGGGSGGGGSGGGGSGGGGSMADVQLVESGGGLVQAGESLRLSCAASGRTLSSLNMAWFRQVPGKERDFVSLISRSGSTDYVHSVRGRFTISRDNAKNTVYLEMNSLKPEDTAVYYCAADDNYNGRYPFRDEYEYWGQGTQVTV

SEQ ID NO: 43 - protein, 278 aa, synthetic construct
DVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVSGGGGSGGGGSGGGGSGGGGSMADVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVS

SEQ ID NO: 44 - protein, 268 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSMAQVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVS

SEQ ID NO: 45 - protein, 268 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSMADVQLVESGGGLVQAGESLRLSCAASGRTLSSLNMAWFRQVPGKERDFVSLISRSGSTDYVHSVRGRFTISRDNAKNTVYLEMNSLKPEDTAVYYCAADDNYNGRYPFRDEYEYWGQGTQVTVS

SEQ ID NO: 46 - protein, 273 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSMADVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVS

SEQ ID NO: 47 - protein, 413 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSMAQVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSMAQVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVS

SEQ ID NO: 48 - protein, 304 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSEAAAKEAAAKEAAAKSGGGGSGGGGSGGGGSGGGGSMAQVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVS

SEQ ID NO: 49 - protein, 289 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSMAQVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSCGSGRCGSGRCGSGRCGSGRC

SEQ ID NO: 50 - protein, 325 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSEAAAKEAAAKEAAAKSGGGGSGGGGSGGGGSGGGGSMAQVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSCGSGRCGSGRCGSGRCGSGRC

SEQ ID NO: 51 - protein, 333 aa, synthetic construct
DVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVSGGGGSGGGGSGGGGSGGGGSDAAARDAAARDAAARGGGGSGGGGSGGGGSGGGGSADVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVSCGSGRCGSGRCGSGRCGSGRC

SEQ ID NO: 52 - protein, 324 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSDAAARDAAARDAAARGGGGSGGGGSGGGGSGGGGSMAQVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSCGSGRCGSGRCGSGRCGSGRC

SEQ ID NO: 53 - protein, 334 aa, synthetic construct
DVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVSGGGGSGGGGSGGGGSGGGGSDAAARDAAARDAAARGGGGSGGGGSGGGGSGGGGSGSMAQVHLVESGGKLVQAGESLKLSCEVSGRTFSISGAGWFRQAPGKEREFVAAITWTGGYPYYDASVKGRFTISREDPDNTVYLSMNSLKPEDTAVYYCATRRAGDLDFSYAQQGRYDNWGQGTQVTVSCGSGRCGSGRCGSGRCGSGRC

SEQ ID NO: 54 - protein, 329 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSDAAARDAAARDAAARGGGGSGGGGSGGGGSGGGGSGSMAQVHLVESGGKLVQAGESLKLSCEVSGRTFSISGAGWFRQAPGKEREFVAAITWTGGYPYYDASVKGRFTISREDPDNTVYLSMNSLKPEDTAVYYCATRRAGDLDFSYAQQGRYDNWGQGTQVTVSCGSGRCGSGRCGSGRCGSGRC

SEQ ID NO: 55 - protein, 332 aa, synthetic construct
QVHLVESGGKLVQAGESLKLSCEVSGRTFSISGAGWFRQAPGKEREFVAAITWTGGYPYYDASVKGRFTISREDPDNTVYLSMNSLKPEDTAVYYCATRRAGDLDFSYAQQGRYDNWGQGTQVTVSGGGGSGGGGSGGGGSGGGGSDAAARDAAARDAAARGGGGSGGGGSGGGGSGGGGSGSMAQVHLVESGGKLVQAGESLKLSCEVSGRTFSISGAGWFRQAPGKEREFVAAITWTGGYPYYDASVKGRFTISREDPDNTVYLSMNSLKPEDTAVYYCATRRAGDLDFSYAQQGRYDNWGQGTQVTVSCGSGRCGSGRCGSGRCGSGRC

SEQ ID NO: 56 - protein, 513 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSDAAARDAAARDAAARGGGGSGGGGSGGGGSGGGGSADVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVSGGGGSGGGGSGGGGSGGGGSDAAARDAAARDAAARGGGGSGGGGSGGGGSGGGGSGSMAQVHLVESGGKLVQAGESLKLSCEVSGRTFSISGAGWFRQAPGKEREFVAAITWTGGYPYYDASVKGRFTISREDPDNTVYLSMNSLKPEDTAVYYCATRRAGDLDFSYAQQGRYDNWGQGTQVTVSCGSGRCGSGRCGSGRCGSGRC

SEQ ID NO: 57 - protein, 513 aa, synthetic construct
QVQLVESGGDLVQAGGSLRLSCAVSGGTSSNYGMGWFRQAPGKEREFVSSISWSGSRTLYSDSVKGRFTISRDNAKNTVDLQMNSLKPEDTAVYYCTAVREYRDYPQRDNFDYWGQGTQVTVSGGGGSGGGGSGGGGSGGGGSDAAARDAAARDAAARGGGGSGGGGSGGGGSGGGGSGSMAQVHLVESGGKLVQAGESLKLSCEVSGRTFSISGAGWFRQAPGKEREFVAAITWTGGYPYYDASVKGRFTISREDPDNTVYLSMNSLKPEDTAVYYCATRRAGDLDFSYAQQGRYDNWGQGTQVTVSGGGGSGGGGSGGGGSGGGGSDAAARDAAARDAAARGGGGSGGGGSGGGGSGGGGSADVQLVESGGGLVQAGGSLRLSCAASGRTDDIYTMGWFRQGPGKEREFVAVISWSITRRSSITHYADSVKGRFTISGENAENTVYLQMNTLKPEDTAVYYCAAKTGYSGSMSSYQDYDSWGQGTQVTVSCGSGRCGSGRCGSGRCGSGRC

SEQ ID NO: 58 - protein, 136 aa, synthetic construct
METGLRWLLLVAVLKGVQCQSVKESGGRLVTPGTPLTLICTVSGFSLSRYAVGWVRQAPGKGLEWIGYIWSGGYTDYANWAKGRFTISKTSTTVDLKITSPTTEDTATYFCAREGDSDDYIYLNLWGPGTLVTVSS

SEQ ID NO: 59 - protein, 132 aa, synthetic construct
MDTRAPTQLLGLLLLWLPGARCALVMTQTPSSVSAAVGGTVTINCQASESISSYLAWYQQKSGQPPKLLIYRASTLASGVPSRFKGSGSGTEFTLTIGDLECADAATYYCQQGYTSFNVDKAFGGGTEVVVK

SEQ ID NO: 60 - protein, 130 aa, synthetic construct
METGLRWLLLLAALQGVQWRSVEESGGRLVTPGTPLKLPCALSGFSLIIFAMIWVRLAPPKGLEWIGGIGINGNACYGRWAKGRFTISQTSSTTVDLKMTNLNTDDTATYFCVLRGFNLWGLGTLITVSS

SEQ ID NO: 61 - protein, 131 aa, synthetic construct
MDTRAPTQLLGLLLLWLPGGICDPVMTQTPSSTSAAVGGTVTINCQSSQTVYKNWLSWFQQKPGQPPKLLIYDVSKLESGVPSRFKGSGSGTQFTLTISGVQCDDAATYYCAGGYSGKIFAFGGGTEVVVK

SEQ ID NO: 62 - protein, 144 aa, synthetic construct
METGLRWLLLVAVLKGVQCQSLEESGGGLVQPEGSLTLTCKASGFSFINNNHICWVRQAPGKGLEWIACIYAGSTGYTYYANWAKGRFTISKTSSTTVTLQMTSLTAADTATYFCARVPYAGYVNYGYTFFNLWGPGTLVTVSS

SEQ ID NO: 63 - protein, 134 aa, synthetic construct
MDTRAPTQLLGLLLLWLPGATFAAVLTQTPSPVSAPVGGTVTISCQSSQSVYRTNDLAWYQQKPGQPPKLLIYWASKLAAGVPSRFKGSGSGTQFTLTISGVQCDDAATYYCLGGYYDDGDLNAFGGGTEVVVN

SEQ ID NO: 64 - protein, 129 aa, synthetic construct
METGLRWLLLVAVLKGVQCQSVEESGGRLVTPGTPLTLTCTVSGIDLSSYVMGWVRQAPGKGLDWIGMIDTHDDTYFAPWARGRFTISRTSSTTVDLKVASTTTEDTATYFCAGNLALWGRGTLVTVSS

SEQ ID NO: 65 - protein, 132 aa, synthetic construct
MDTRAPTQLLGLLLLWLPGATFAQVLTQTPSPVSVAVGGTVTISCQSSQSVSSDNWLGWYQQKPGQPPKLLIYRASKLASGVSSRFKGSGSGTQFILTISDLECDDAATYYCAGGYSGSIIAFGGGTEVVVK

SEQ ID NO: 66 - protein, 136 aa, synthetic construct
METGLRWLLLVAVLKGVQCQSVEESGGRLVTPGTPLTLTCTVSGFSLSTYAMGWFRQAPGKGLEWIGTIWSGAFTDYANWVNGRFTISKTSTTVDLKITSPTTEDTATYFCAREADSDLYTYFKLWGPGTLVTVSS

SEQ ID NO: 67 - protein, 132 aa, synthetic construct
MDTRAPTQLLGLLLLWLPGARCAYDMTQTPASVEVAVGGTVTLKCQASESIEIYLAWYQQKPGQPPKLLVYDASTLASGVPSRFKGSGSGTQFTLTISGVECADAATYYCQQGYRSDNVHNVFGGGTEVVVK

SEQ ID NO: 68 - protein, 140 aa, synthetic construct
METGLRWLLLVAVLKGVQCQSLEESGGDLVKPGASLTLTCTASGFSLNNYVMYWVRQAPGKGLESIACIYTGSGTTYYATWAKGRFTISKTSSTTVTLQMTSLTAADTATYFCARRTYTSDSYAGSFNLWGPGTLVTVSS

SEQ ID NO: 69 - protein, 132 aa, synthetic construct
MDTRAPTQLLGLLLLWLPGARCAYDMTQTPASVSEPVGGTVTIKCQASQSIGSNLAWYQQKPGQRPKLLIYAASYLASGAPSRFKGSGSGTEFTLTISGVQCDDAATYYCQQGWSSSDVDSTFGGGTEVVVK

SEQ ID NO: 70 - protein, 135 aa, synthetic construct
METGLRWLLLVAVFKGVQCQSVEESGGRLVTPGTPLTLTCTVSGFSLSRHYMSWVRQAPGKGLEWIGIIYDDDNRDHASWAKGRFTISRTSTTVDLKITSPTTEDTATYFCARVYASYGYALNLWGQGTLVIVSS

SEQ ID NO: 71 - protein, 132 aa, synthetic construct
MDTRAPTQLLGLLLLWLPGARCALVMTQTPPSVSAAVGGTVTINCQASESITNWLAWYQQKPGQPPKLLIYDASKLASGVSSRFSGSGSGTQFTLTISRVQCDDAATYYCQQGYTWNNIDNVFGGGTEVVVK

SEQ ID NO: 72 - protein, 136 aa, synthetic construct
METGLRWLLLVAVLKGVQCQSVEESGGRLVKPDESLTLTCTVSGFSLSAYEMGWVRQAPGKGLEWIAAISTGDTPYYATWTKGRFTISRASSTTVDLKMTSPTTEDTATYFCARYGHNYEYVFDLWGQGTLVTVSS

SEQ ID NO: 73 - protein, 132 aa, synthetic construct
MDTRAPTQLLGLLLLWLPGARCAYDMTQTPASVSAAVGGTVTINCQASQSIGSYLSWYQQKPGQPPKLLIYSASTLASGVPSRFSGSGYGTEFTLTIGGVQCDDAAAYYCQQGESIGSVHNVFGGGTEVVVA

SEQ ID NO: 74 - protein, 144 aa, synthetic construct
METGLRWLLLVAVLKGVQCQSLEESGGDLVKPGASLTLTCTASGFSFINNNHICWVRQAPGKGLEWIACIYAGSNGYTYYTNWAKGRFTISKTSSTTVTLQMTSLTAADTATYFCARVPYAGYVNYGYAFFNLWGPGTLVTVSS

SEQ ID NO: 75 - protein, 134 aa, synthetic construct
MDTRAPTQLLGLLLLWLPGATFAAVVTQTPSPVSAPVGGTVTISCQSSQSVYRTNDLAWYQQKPGQPPKLLIYWASKLAIGVPSRFKGSGSGTQFTLTISGVQCDDAATYYCLGGYDDDGDLNTFGGGTEVVVN

SEQ ID NO: 76 - protein, 136 aa, synthetic construct
METGLRWLLLVAVLKGVQCQSVEESGGRLVTPGTPLTLTCTVSGFSLSSYAMTWVRQAPGKGLEWIGYIYAGGIADYANWAKGRFTISKTSTTVDLTITSPTTEDTATYFCAREGDSDDYVYLSLWGPGTLVTVSS

SEQ ID NO: 77 - protein, 132 aa, synthetic construct
MDTRAPTQLLGLLLLWLPGARCALVMTQTPSSVSAAVGGTVTINCQASQSIVGYLAWYQQKPGQPPKLLIYRASTLASGVSSRFKGSGSGTEFTLTISGVQCADAATYYCQQGYINSNIDNTFGGGTEVVVK

SEQ ID NO: 78 - protein, 338 aa, Homo sapiens
MGKKQNRKTGNSKTQSASPPPKERSSSPATEQSWMENDFDELREEGFRRSNYSELREDIQTKGKEVENFEKNLEECITRITNTEKCLKELMELKTKARELREECRSLRSRCDQLEERVSAMEDEMNEMKREGKFREKRIKRNEQSLQEIWDYVKRPNLRLIGVPESDVENGTKLENTLQDIIQENFPNLARQANVQIQEIQRTPQRYSSRRATPRHIIVRFTKVEMKEKMLRAAREKGRVTLKGKPIRLTADLSAETLQARREWGPIFNILKEKNFQPRISYPAKLSFISEGEIKYFIDKQMLRDFVTTRPALKELLKEALNMERNNRYQPLQNHAKM

SEQ ID NO: 79 - DNA, 408 bp, synthetic construct
  source 1..408: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcaccgtctctggattctccctcagtagctatgcaatgacctgggtccgccaggctccagggaaggggctggaatggatcggatacatttatgctggtggtattgcggactacgcgaactgggcgaaaggccgattcaccatctccaaaacctcgaccacggtggatctgacaatcaccagtccgacaaccgaggacacggccacctatttctgtgccagagagggggatagtgatgattatgtttatcttagcttgtggggcccaggcaccctggtcaccgtctcctca

SEQ ID NO: 80 - DNA, 396 bp, synthetic construct
  source 1..396: mol_type = other DNA
atggacacgagggcccccactcagctgctggggctcctgctgctctggctcccaggtgccagatgtgcccttgtgatgacccagactccatcctccgtgtctgcagctgtgggaggcacagtcaccatcaattgccaggccagtcagagcattgttggctacttagcctggtatcagcagaaaccagggcagcctcccaagctcctgatctacagggcatccactctggcatctggggtctcatcgcggttcaaaggcagtggatctgggacagagttcactctcaccatcagcggtgtgcagtgtgccgatgctgccacttactactgtcaacagggttatattaatagtaatattgataatactttcggcggagggaccgaggtggtggtcaaa

SEQ ID NO: 81 - DNA, 390 bp, synthetic construct
  source 1..390: mol_type = other DNA
atggagactgggctgcgctggcttctcctgctcgctgcgctccaaggagttcagtggcggtcggtggaggagtccggaggacgcctggtcacgcctgggacacccttgaaactcccctgcgctctctctggattctccctcattatctttgcaatgatctgggtccgcctggctccaccgaaggggctggaatggattggagggattggtatcaatggtaatgcatgctacgggaggtgggcgaaagggcgattcaccatctcccaaacctcctcgaccaccgtggatctgaaaatgaccaatctgaacaccgatgacacggccacatatttctgtgtattacggggttttaacttgtggggactagggaccctgatcaccgtctcctca

SEQ ID NO: 82 - DNA, 393 bp, synthetic construct
  source 1..393: mol_type = other DNA
atggacacgagggcccccactcagctgctggggctcctgctgctctggctcccaggtggcatatgtgaccctgtgatgacccagactccatcttccacgtctgcggctgtgggaggcacagtcaccatcaactgccagtccagtcagactgtttataagaactggttatcctggtttcagcagaaaccagggcagcctcccaagctcctgatttatgatgtatcaaaactggaatcaggggtcccatcccggtttaaaggcagtggatctgggacacagttcactctcaccatcagcggcgtgcagtgtgacgatgctgccacttactactgtgcaggcggttatagtggtaaaatttttgctttcggcggagggaccgaggtggtggtcaaa

SEQ ID NO: 83 - DNA, 420 bp, synthetic construct
  source 1..420: mol_type = other DNA
atggagactgggctgcgctggcttctcctggtcgctgtgctcaaaggtgtccagtgtcagtcgttggaggagtccgggggagacctggtcaagcctggggcttccctgacactcacctgcacagcctctggattctccctcaataattatgtaatgtactgggtccgccaggctccagggaaggggctggagtcgatcgcatgcatttatactggtagtggtactacttactacgcgacctgggcgaaaggccgattcaccatctccaaaacctcgtcgaccacggtgactctgcaaatgaccagtctgacagccgcggacacggccacctatttctgtgcgagacgtacttatactagtgatagttatgcgggatcttttaatttgtggggcccaggcaccctggtcaccgtctcctca

SEQ ID NO: 84 - DNA, 396 bp, synthetic construct
  source 1..396: mol_type = other DNA
atggacacgagggcccccactcagctgctggggctcctgctgctctggctcccaggtgccagatgtgcctatgatatgacccagactccagcctccgtgtctgaacctgtgggaggcacagtcaccatcaagtgccaggccagtcagagcattggtagtaatttagcctggtatcagcagaaaccagggcagcgtcccaagctcctgatctatgctgcatcctatttggcatctggggccccatcacggttcaaaggcagtggatctgggacagagttcactctcaccatcagcggcgtgcagtgtgacgatgctgccacttactactgtcaacagggttggagtagtagtgatgtggatagtactttcggcggagggaccgaggtggtggtcaaa

SEQ ID NO: 85 - DNA, 408 bp, synthetic construct
  source 1..408: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtacctatgcaatgggctggttccgccaggctccagggaaggggctggagtggatcgggaccatttggagtggtgctttcacggactacgcaaattgggtgaatggccgattcaccatctccaaaacctcgaccacggtggatctgaaaatcactagtccgacaaccgaggacacggccacctatttctgtgccagagaagctgatagtgatctttatacctactttaaattgtggggcccaggcaccctggtcaccgtctcctca

SEQ ID NO: 86 - DNA, 396 bp, synthetic construct
  source 1..396: mol_type = other DNA
atggacacgagggcccccactcagctgctggggctcctgctgctctggctcccaggtgccagatgtgcctatgatatgacccagactccagcctctgtggaggtagctgtgggaggcacagtcaccctcaagtgccaggccagtgagagcattgaaatctatttagcctggtatcagcagaaaccagggcagcctcccaagctcctggtctatgatgcatccactctggcatctggggtcccatcgcggttcaaaggcagtggatctgggacacagttcactctcaccatcagcggcgtggagtgtgccgatgctgccacttactactgtcaacagggttatagaagtgataatgttcataatgttttcggcggagggaccgaggtggtggtcaaa

SEQ ID NO: 87 - DNA, 405 bp, synthetic construct
  source 1..405: mol_type = other DNA
atggagactgggctgcgctggcttctcctggtcgctgtgttcaaaggtgtccagtgtcagtcggtggaggagtccgggggtcgcctggtcacgcctgggacacccctgacactcacctgcacagtctctggattctccctcagtaggcactatatgagttgggtccgccaggctccagggaaggggctggaatggatcggaatcatttatgatgatgataatagagatcacgcgagctgggcgaaaggccgattcaccatctccagaacctcgaccacggtggatctgaaaatcaccagtccgacaaccgaggacacggccacctatttctgtgccagagtttatgctagttatggttatgcacttaacttgtggggccaaggcaccctggtcatcgtctcctca

SEQ ID NO: 88 - DNA, 396 bp, synthetic construct
  source 1..396: mol_type = other DNA
atggacacgagggcccccactcagctgctggggctcctgctgctctggctcccaggtgccagatgtgcccttgtgatgacccagactccaccctcggtgtctgcagctgtgggaggcacagtcaccatcaattgccaggcaagtgagagcattaccaactggttagcctggtatcagcagaaaccagggcagcctcccaagctcctgatctacgatgcgtcgaaactggcatctggggtctcatcgcggttcagtggcagtggatctgggacacagttcactcttaccatcagcagagtgcagtgtgacgatgctgccacttactactgtcaacagggttatacttggaataatattgataatgttttcggcggagggaccgaggtggtggtcaaa

SEQ ID NO: 89 - DNA, 408 bp, synthetic construct
  source 1..408: mol_type = other DNA
atggagactgggctgcgctggcttctcctggtcgctgtgctcaaaggtgtccagtgtcagtcggtggaggagtccgggggtcgcctggtaaagcctgacgaatccctgacactcacctgcacagtctctggattctccctcagtgcctatgaaatgggctgggtccgccaggctccagggaaggggctggagtggatcgcagccattagtactggtgataccccatactacgcgacctggacaaaaggccgattcaccatctccagggcctcgtcgaccacggtggatctgaaaatgaccagtccgacaaccgaggacacggccacctatttctgtgccagatatggtcataattatgaatacgtctttgacttgtggggccaaggcaccctggtcaccgtctcctca

SEQ ID NO: 90 - DNA, 396 bp, synthetic construct
  source 1..396: mol_type = other DNA
atggacacgagggcccccactcagctgctggggctcctgctgctctggctcccaggtgccagatgcgcctatgatatgacccagactccagcctccgtgtctgcagctgtgggaggcacagtcaccatcaattgccaggccagtcagagcattggtagttacttatcctggtatcagcagaaaccagggcagcctcccaagctcctgatctattctgcatccactctggcatctggggtcccatcgcggttcagtggcagtggatatgggacagagttcactctcaccatcggcggtgtgcagtgtgacgatgctgccgcttactactgtcaacagggtgagagtattggcagtgttcataatgttttcggcggagggaccgaggtggtggtcgca

SEQ ID NO: 91 - DNA, 432 bp, synthetic construct
  source 1..432: mol_type = other DNA
atggagactgggctgcgctggcttctcctggtcgctgtgctcaaaggtgtccagtgtcagtcgttggaggagtccgggggagacctggtcaagcctggggcatccctgacactcacctgcacagcctctggattctccttcattaacaacaatcacatctgttgggtccgtcaggctccagggaaggggctggagtggatcgcatgcatttatgctggtagtaatggttacacttactacacaaactgggcgaaaggccgattcaccatctccaaaacctcgtcgaccacggtgactctgcaaatgaccagtctgacagccgcggacacggccacctatttctgtgcgagagttccctatgctggttatgttaattatggctatgccttctttaacttgtggggcccaggcaccctggtcaccgtctcctca

SEQ ID NO: 92 - DNA, 402 bp, synthetic construct
  source 1..402: mol_type = other DNA
atggacacgagggcccccactcagctgctggggctcctgctgctctggctcccaggtgccacatttgcagccgtggtgacccagacaccatcacccgtgtctgcacctgtgggaggcacagtcaccatcagttgccagtccagtcagagtgtttataggaccaacgacttggcctggtatcagcagaaaccagggcagcctcccaaactcctcatctactgggcatccaaactggcaattggggtcccatcgcggttcaaaggcagtgggtctgggacacagtttactctcaccatcagcggcgtgcagtgtgacgatgctgccacttactactgtctaggcggttatgatgatgatggtgatcttaatactttcggcggagggaccgaggtggtggtcaac

SEQ ID NO: 93 - DNA, 432 bp, synthetic construct
  source 1..432: mol_type = other DNA
atggagactgggctgcgctggcttctcctggtcgctgtgctcaaaggtgtccagtgtcagtcgttggaggagtccgggggaggcctggtccagcctgagggatccctgacactcacctgcaaagcctctggattctccttcattaacaacaatcacatctgttgggtccgtcaggctccagggaaggggctggagtggatcgcatgcatttacgctggtagtactggctacacttactacgcgaactgggcgaaaggccgattcaccatctccaaaacctcgtcgaccacggtgactctgcaaatgaccagtctgacagccgcggacacggccacctatttctgtgcgagagttccctatgctggttatgttaattatggctataccttctttaacttgtggggcccaggcaccctggtcaccgtctcctca

SEQ ID NO: 94 - DNA, 402 bp, synthetic construct
  source 1..402: mol_type = other DNA
atggacacgagggcccccactcagctgctggggctcctgctgctctggctcccaggtgccacatttgcagccgtgctgacccagacaccatcacccgtgtctgcacctgtgggaggcacagtcaccatcagttgccagtccagtcagagtgtttataggaccaacgacttggcctggtatcagcagaaaccagggcagcctcccaaactcctgatctactgggcatccaaactggcagctggggtcccatcacggttcaaaggcagtggatctgggacacagttcactctcaccatcagcggcgtgcagtgtgacgatgctgccacttactactgtctaggcggttattatgatgatggtgatcttaatgctttcggcggagggaccgaggtggtggtcaac

SEQ ID NO: 95 - DNA, 408 bp, synthetic construct
  source 1..408: mol_type = other DNA
atggagactgggctgcgctggcttctcctggtcgctgtgctcaaaggtgtccaatgtcagtcggtgaaggagtccgggggtcgcctggtcacgcctgggacacccctgacactcatttgcacagtctctggattctccctcagtagatatgcagtgggctgggtccgccaggctccagggaaggggctggagtggatcggatacatttggagtggtggttacacggactacgcgaactgggcgaaaggccgattcaccatctccaaaacctcgaccacggtggatctgaaaatcaccagtccgacaaccgaggacacggccacctatttctgtgccagagagggggatagtgatgattatatttatcttaacttatggggcccaggcaccctggtcaccgtctcctca

SEQ ID NO: 96 - DNA, 396 bp, synthetic construct
  source 1..396: mol_type = other DNA
atggacacgagggcccccactcagctgctggggctcctgctgctctggctcccaggtgccagatgtgcccttgtgatgacccagactccatcctccgtgtctgcagctgtgggaggcacagtcaccatcaattgccaggccagtgagagcattagtagttacttagcctggtatcagcagaaatcagggcagcctcccaagctcctgatctatagggcatccactctggcatctggggtcccatcacggttcaaaggcagtggatctgggacagagttcactctcaccatcggcgacctggagtgtgccgatgctgccacttactactgtcaacagggttatactagttttaatgttgataaagctttcggcggagggaccgaggtggtggtcaaa

SEQ ID NO: 97 - DNA, 387 bp, synthetic construct
  source 1..387: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagctatgtaatgggctgggtccgccaggctccagggaaggggctggattggatcggaatgattgatacacatgatgatacatatttcgcgccctgggcgagaggccgattcaccatctccagaacctcgtcgaccacggtggatctgaaggtggccagtacgacaaccgaggacacggccacctatttctgtgccggaaatcttgccttgtggggccgaggcaccctggtcaccgtctcctcg

SEQ ID NO: 98 - DNA, 396 bp, synthetic construct
  source 1..396: mol_type = other DNA
atggacacgagggcccccactcagctgctggggctcctgctgctctggctcccaggtgccacatttgctcaagtgctgacccagactccatcgcccgtgtctgtagctgtgggaggcacagtcaccatcagttgccagtccagtcagagtgtctctagtgacaactggttaggctggtatcagcagaaaccagggcagcctcccaagctcctgatctacagggcatccaagctggcatctggggtctcatcgcggttcaaaggcagtggatctgggacacagttcattcttactattagtgacctggagtgtgacgatgctgccacttactactgtgcaggcggttatagtggtagtattattgctttcggcggagggaccgaggtggtggtcaaa

SEQ ID NO: 99 - protein, 20 aa, synthetic construct
GGGGSGGGGSGGGGSGGGGS

SEQ ID NO: 100 - protein, 15 aa, synthetic construct
EAAAKEAAAKEAAAK

SEQ ID NO: 101 - protein, 15 aa, synthetic construct
DAAARDAAARDAAAR

SEQ ID NO: 102 - protein, 21 aa, synthetic construct
CGSGRCGSGRCGSGRCGSGRC